(19) United States
(12) Patent Application Publication
(10) Pub. No.: US 2010/0261876 A1
(43) Pub. Date: Oct. 14, 2010

(54) NOVEL METHODS OF SYNTHESIS FOR THERAPEUTIC ANTIVIRAL PEPTIDES

(76) Inventors: Brian L. Bray, Graham, NC (US); Barbara E. Johnston, Raleigh, NC (US); Stephen E. Schneider, Raleigh, NC (US); Nicolai A. Tvermoes, Durham, NC (US); Huyi Zhang, Durham, NC (US); Paul E. Friedrich, Apex, NC (US)

Correspondence Address: JONES DAY, 222 EAST 41ST ST, NEW YORK, NY, 10017, US

(21) Appl. No.: 12/679,609

(22) PCT Filed: Sep. 25, 2008
(86) PCT No.: PCT/US08/11147

§ 371 Date: Jun. 15, 2010

Related U.S. Application Data
(60) Provisional application No. 60/995,318, filed on Sep. 25, 2007.

(51) Int. Cl. C07K 14/00; C07K 7/08; C07K 7/06; C07K 5/072

(52) U.S. Cl. 530/324; 530/325; 530/326; 530/327; 530/328; 562/561

(57) ABSTRACT

Provided herein are methods for synthesis of peptides. In particular, provided herein are methods of synthesis for therapeutic antiviral peptides.

### CROSS-REFERENCE TO RELATED APPLICATION

This application is a national stage application and claims the benefit of International Application No. PCT/US2008/011147, filed Sep. 25, 2008, which claims priority to U.S. Provisional Application No. 60/995,318, filed Sep. 25, 2007. The disclosure of each of these applications being incorporated by reference herein in their entireties.

### FIELD

Provided herein are peptides and compositions comprising the peptides, and methods for the synthesis of peptides. Also provided are the methods for administrations of the therapeutic agents. In particular, provided herein are methods for the synthesis of SEQ. ID NO:9 (TRI-1144) and similar peptides. Such methods utilize solid and liquid phase synthesis procedures to synthesize and combine groups of specific peptide fragments to yield a peptide of interest. Also provided herein are individual peptides which act, for example, as intermediates in the synthesis of the peptides of interest, e.g., SEQ ID NO:9. Still further presented herein are groups of peptides which can be used together to produce full-length SEQ ID NO:9 and similar peptides.

### BACKGROUND

### Peptide Delivery Systems

Peptide products have a wide range of uses as therapeutic and/or prophylactic agents for prevention and treatment of disease. Such peptide products fall into diverse categories such as, for example, hormones, enzymes and immunomodulators (e.g., antibodies, serum proteins and cytokines).

For peptides to manifest their proper biological and therapeutic affect in patients, they must be present in appropriate concentrations at their sites of action in vivo. More specifically, the pharmacokinetics of any particular compound, including any particular peptide, is dependent on the bioavailability, distribution and clearance of that compound in vivo. However, the chemical nature and characteristics of peptides, such as size, complexity, conformational requirements and solubility profiles, tend to cause peptides to have pharmacokinetic profiles that are suboptimal compared to the pharmacokinetic profiles of other compounds.

Accordingly, there has been considerable effort in the art to attempt to develop ways to administer therapeutic agents such as peptides so that both the bioavailability and the half-life of the therapeutic agents are increased. While progress has been made in this regard, there a remains a need in the art for additional compositions useful for delivering peptide therapeutics with desirable pharmacokinetic profiles. The compositions and methods provided herein address these needs.

### SUMMARY

Provided herein are compositions which can, for example, be used to administer bioactive molecules to a patient, and methods for synthesis of these compositions. Specifically, provided herein are compositions comprising a solvent, a gelling material and a bioactive molecule, such as an antiviral peptide. Further provided herein are methods of synthesis comprising linear synthesis, 2 fragment and 3 fragment approaches to produce compositions, including an antiviral peptide. Without being limited by theory, the embodiments provided herein are based, at least in part, on the unexpected discovery that an increased weight percent of a bioactive molecule can be incorporated in the compositions while exhibiting a desirable pharmacokinetic profile upon administration to a subject. The embodiments provided herein are also based, at least in part, on the unexpected results from synthesis methods, for example, on dimensions such as scalability and purity.

In one embodiment, upon administration to a patient, the compositions provided herein yield plasma concentrations of a biomolecule that quickly (e.g., within 8, 12, 16, 20, 24, 28, 32, 36 or 48 hours) reach Cₘₐₓ and then provide relatively constant plasma concentrations of the biomolecule for 5, 7, 10, 14, 17, 21 or 28 days or longer. In a particular embodiment, desirable pharmacokinetic properties for the compositions provided herein are a lower Cₘₐₓ, a longer tₘₐₓ and a longer t_{0.01} or t_{0.1}.

The compositions provided herein are, for example, useful for administering compositions comprising certain antiviral peptides, referred to as T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9), or a combination of two or more of these peptides, as well as derivatives of the T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9) peptides.

In one embodiment, the compositions comprise a solvent, a gelling material that forms a matrix upon solvent-subcutaneous fluid exchange, and at least one bioactive molecule, e.g., an antiviral peptide such as T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9) or a derivative thereof.

In another embodiment, such compositions further comprise at least one additional component such as a pharmaceutically acceptable carrier, a macromolecule, or a combination thereof. In yet another embodiment, such compositions can further comprise an antiviral agent in addition to the antiviral peptides listed above.

Further provided herein are methods of using the compositions provided herein. In one embodiment, the compositions are used as a part of a therapeutic regimen, for example, an antiviral therapeutic regimen. In certain embodiments, such a therapeutic regimen can, for example, be used for the therapy of HIV infection, e.g., HIV-1 infection.

In one embodiment, provided herein is a method of using the compositions provided herein for inhibition of transmission of HIV to a target cell, comprising administering an amount of a composition provided herein to a patient such that the target cell is contacted with an amount of an active agent, e.g., an antiviral peptide and/or another antiviral agent, effective to inhibit infection of the cell by the virus.

Also provided herein are methods of treating HIV infection (in one embodiment, HIV-1 infection) comprising administering to an HIV-infected patient a composition provided herein in an amount effective to treat the HIV infection.

Further provided herein are methods for the use of a composition containing an effective amount of a bioactive molecule, such as an antiviral peptide, in the manufacture of a medicament for use in therapy of HIV infection (e.g., used in a method of inhibiting transmission of HIV, a method of inhibiting HIV fusion, and/or a method of treating or inhibiting HIV infection).

The above and other objects, features, and advantages of the compositions and methods provided herein will be apparent in the following Detailed Description when read in conjunction with accompanying drawings.

### BRIEF DESCRIPTION OF THE DRAWINGS

FIG. 1 is a schematic of HIV-1 gp41 showing the heptad repeat 1 region (HR1) and heptad repeat 2 region (HR2), which includes the well-known leucine zipper-like motif INNYTSLI, along with other functional regions of gp41. Exemplary natural amino acid sequences corresponding to HR1 and HR2, and the amino acid position numbering, are shown for purposes of illustration and in relation to gp160, strain HIV_{IIIB}.

FIG. 2 shows a comparison of the natural amino acid sequences contained within the HR2 region of HIV-1 gp41 for purposes of illustration, and not limitation, as determined from various laboratory strains and clinical isolates, wherein illustrated are some of the variations in amino acid sequence (e.g., polymorphisms), as indicated by the single letter amino acid code. For purposes of example, those isolate sequences that align with the amino acid sequence INNYTSLI in the top isolate sequence correspond to HR2 leucine zipper-like motifs.

FIG. 3 is a schematic showing synthesis of a peptide having an amino acid sequence of SEQ ID NO:9, using a fragment condensation approach involving assembly of 2 peptide fragments.

FIG. 4 is a schematic showing synthesis of a peptide having an amino acid sequence of SEQ ID NO:9, using a rink-loaded CTC 2 fragment condensation assembly strategy.

FIG. 5 is a schematic showing synthesis of an HIV fusion inhibitor peptide having an amino acid sequence of SEQ ID NO:9, applying the use of Sieber resin to a 2 fragment condensation approach.

FIG. 6 is a schematic showing synthesis of an HIV fusion inhibitor peptide having an amino acid sequence of SEQ ID NO:9, using Glu-side chain loaded resin in an approach involving assembly of 2 peptide fragments.

FIG. 7 is a schematic showing synthesis of an HIV fusion inhibitor peptide-SEQ ID NO:9, having an amino acid sequence of SEQ ID NO:9, using a fragment condensation approach involving assembly of 3 peptide fragments.

FIG. 8 is a schematic showing synthesis of an HIV fusion inhibitor peptide-SEQ ID NO:9, having an amino acid sequence of SEQ ID NO:9, using a fragment condensation approach involving assembly of 2 peptide fragments.

FIG. 9 shows a plot of T1144 plasma concentrations in cynomolgus monkeys over a 432 hour period postdose for T1144 administered in the following compositions: 1000 μl of a 100 mg/g suspension of T1144 precipitated by ZnSO₄ solution (89% peptide) in 74:11:15 SAIB:PLA3L:NMP (--♦--) and 400 -) of a 50 mg/g suspension of T1144 precipitated by ZnSO₄ solution (60% peptide) in 40:60 PLA3L:NMP (--▪--).

FIG. 10 shows a plot of T1144 plasma concentrations in rats over a 168 hour period postdose for T1144 administered in the following compositions: 400 μl of a 100 mg/g suspension of T1144 precipitated by ZnSO₄ solution (89% peptide) in 74:11:15 SAIB:PLA3L:NMP (--♦--) and 400 μl of a 50 mg/g suspension of T1144 precipitated by ZnSO₄ solution (60% peptide) in 40:60 PLA3L:NMP (--▪--).

FIG. 11 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (89% peptide, 2% Zinc) in 80:0:20 SAIB:PLA3M:NMP (--♦--), 400 μl of a 50 mg/g suspension of T1144 precipitated by ZnSO₄ solution (89% peptide, 2% Zinc) in 75:5:20 SAIB:PLA3M:NMP (--▪--) and 400 μl of a 50 mg/g suspension of T1144 precipitated by ZnSO₄ solution (89% peptide, 2% Zinc) in 65:15:20 SAIB:PLA3M:NMP (--▴--).

FIG. 12 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 85:0:15 SAIB:PLA3M:NMP (--♦--) and 400 μl of a 50 mg/g suspension of T1144 precipitated by ZnSO₄ solution (73% peptide, 2% Zinc) in 74:11:15 SAIB:PLA3M:NMP (--▪--).

FIG. 13 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 70:10:20 SAIB:PLA3M:NMP (--♦--) and 400 μl of a 50 mg/g suspension of T1144 precipitated by ZnSO₄ solution (73% peptide, 2% Zinc) in 75:5:20 SAIB:PLA3M:NMP (--▪--).

FIG. 14 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 70:10:20 SAIB:PLA3M:NMP (--♦--) and 400 μl of a 50 mg/g suspension of T1144 precipitated by ZnSO₄ solution (73% peptide, 2% Zinc) in 74:11:15 SAIB:PLA3M:NMP (--▪--).

FIG. 15 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 75:5:20 SAIB:PLA3M:Triacetin (--♦--), 400 μl of a 50 mg/g suspension of T1144 precipitated by ZnSO₄ solution (73% peptide, 2% Zinc) in 75:5:20 SAIB:PLA3M:BenzylBenzoate (--▪--) and 400 μl of a 50 mg/g suspension of T1144 precipitated by ZnSO₄ solution (73% peptide, 2% Zinc) in 75:5:20 SAIB:PLA3M:NMP (--▴--).

FIG. 16 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 75:5:20 SAIB:PLA3M:NMP (--♦--), 400 μl of a 75 mg/g suspension of T1144 precipitated by ZnSO₄ solution (73% peptide, 2% Zinc) in 75:5:20 SAIB:PLA3M:NMP (--▪--) and 400 μl of a 100 mg/g suspension of T1144 precipitated by ZnSO₄ solution (73% peptide, 2% Zinc) in 75:5:20 SAIB:PLA3M:NMP (--▴--).

FIG. 17 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 precipitated by ZnSO₄ solution (88% peptide, 2% Zinc) in 77:15:8 SAIB:NMP:Ethanol (--♦--), 200 μl of a 100 mg/g suspension of T1144 precipitated by ZnSO₄ solution (88% peptide, 2% Zinc) in 77:15:8 SAIB:NMP:Ethanol (--▪--), 400 μl of a 100 mg/g suspension of T1144 precipitated by ZnSO₄ solution (73% peptide, 2% Zinc) in 74:11:15 SAIB:PLA3M:NMP (--⋄--) and 200 μl of a 100 mg/g suspension of T1144 precipitated by ZnSO₄ solution (73% peptide, 2% Zinc) in 74:11:15 SAIB:PLA3M:NMP (--□--).

FIG. 18 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (89% peptide, 2% Zinc) in 75:5:20 SAIB:PLA3L:NMP (--♦--) and 400 μl of a 50 mg/g suspension of T1144 precipitated by ZnSO₄ solution (89% peptide, 2% Zinc) in 75:5:20 SAIB:PLA3M:NMP (--▪--).

FIG. 19 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 precipitated by ZnSO₄ solution (73%) in 75:5:20 SAIB:PLA3L:NMP (--♦--), 400 μl of a 50 mg/g suspension of T1144 precipitated by ZnSO₄ solution (89%) in 75:5:20 SAIB:PLA3L:NMP (--▪--), 400 μl of a 50 mg/g suspension of T1144 precipitated by ZnSO₄ solution (73%) and slurried in ZnSO₄ solution (70%) in 75:5:20 SAIB:PLA3L:NMP (--▴--), 400 μl of a 50 mg/g suspension of T1144 spray dried (89%) and slurried in ZnSO₄ solution (66%) in 75:5:20 SAIB:PLA3L:NMP (--∘--), 400 μl of a 50 mg/g suspension of T1144 precipitated by ZnSO₄ solution (88%) and slurried in ZnSO₄ solution (71%) in 75:5:20 SAIB:PLA3L:NMP (-- [symbol] --) and 400 μl of a 50 mg/g suspension of T1144 spray dried (89%) and slurried in ZnSO₄ solution (65%) in 75:5:20 SAIB:PLA3L:NMP (--x--).

FIG. 20 shows a plot of T1144 plasma concentrations in cynomolgus monkeys over a 432 hour period postdose for T1144 administered in the following compositions: 800 μl of a 3.5 mg/mL solution of T1144 (-), 400 μl of a 50 mg/g suspension of T1144 precipitated by ZnSO₄ solution (89%) in 75:5:20 SAIB:PLA3M:NMP (--♦--), 400 μl of a 100 mg/g suspension of T1144 precipitated by ZnSO₄ solution (89%) in 74:11:15 SAIB:PLA3L:NMP (--▪--) and 1000 μl of a 100 mg/g suspension of T1144 precipitated by ZnSO₄ solution (89%) in 74:11:15 SAIB:PLA3L:NMP (--▴--).

FIG. 21 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 precipitated by ZnSO₄ solution (89% peptide, 2% Zinc) in 40:60 PLGA1A:NMP (--♦--), 400 μl of a 50 mg/g suspension of T1144 precipitated by ZnSO₄ solution (89% peptide, 2% Zinc) in 60:40 PLGA1A:NMP (--▪--) and 400 μl of a 50 mg/g suspension of T1144 precipitated by ZnSO₄ solution (88% peptide, 2% Zinc) in 40:60 PLA3L:NMP (--▴--).

FIG. 22 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 precipitated by ZnSO₄ solution (73% peptide, 2% Zinc) in 50:50 PLGA1A:NMP (--♦--), 400 μl of a 50 mg/g suspension of T1144 precipitated by ZnSO₄ solution (73% peptide, 2% Zinc) in 40:60 PLGA1A:Triacetin (--▪--), 400 μl of a 50 mg/g suspension of T1144 precipitated by ZnSO₄ solution (73% peptide, 2% Zinc) in 40:60 PLGA3A:NMP (--▴--) and 400 μl of a 50 mg/g suspension of T1144 precipitated by ZnSO₄ solution (73% peptide, 2% Zinc) in 40:60 PLA3L:NMP (--∘--).

FIG. 23 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by ZnSO₄ solution (73% peptide, 2% Zinc) in 50:50 PLGA1A:NMP (--♦--), 400 μl of a 100 mg/g suspension of T1144 precipitated by ZnSO₄ solution (73% peptide, 2% Zinc) in 50:50 PLGA1A:NMP (--▪--), 400 μl of a 50 mg/g suspension of T1144 precipitated by ZnSO₄ solution (91% peptide, 2% Zinc) in 40:60 PLA3L:NMP (--⋄--), 400 μl of a 100 mg/g suspension of T1144 precipitated by ZnSO₄ solution (91% peptide, 2% Zinc) in 40:60 PLA3L:NMP (--□--) and 400 μl of a 200 mg/g suspension of T1144 precipitated by ZnSO₄ solution (90% peptide, 2% Zinc) in 40:60 PLA3L:NMP (--Δ--).

FIG. 24 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 prepared by spray drying in 40:60 PLA3L:NMP (--♦--) and 400 μl of a 50 mg/g suspension of T1144 pH precipitated in MeOH (88% peptide) 40:60 PLA3L:NMP (--▪--).

FIG. 25 shows a plot of T1144 plasma concentrations in rats over a 168 hour period postdose for T1144 administered in the following compositions: 1200 μl of a 3 mg/mL solution of T1144 (-), 400 μl of a 50 mg/g suspension of T1144 precipitated by ZnSO₄ solution (60%) in 40:60 PLA3L:NMP (--♦--), 400 μl of a 50 mg/g suspension of T1144 washed (94%) in 40:60 PLA3L:NMP (--▪--), 400 μl of a 50 mg/g suspension of T1144 precipitated by ZnSO₄ solution (88%) in 40:60 PLA3L:NMP (--▴--) and 400 μl of a 50 mg/g suspension of T1144 precipitated from MeOH/H₂O (91%) in 40:60 PLA3L:NMP (--∘--).

FIG. 26 shows a plot of T1144 plasma concentrations in rats over a 168 hour period postdose for T1144 administered in the following compositions: 1200 μl of a 3 mg/mL solution of T1144 (-) 400 μl of a 50 mg/g suspension of T1144 precipitated by CaCl₂ solution (29%) in 40:60 PLA3L:NMP (--♦--), 400 μl of a 50 mg/g suspension of T1144 precipitated by CaCl₂ solution (53%) in 40:60 PLA3L:NMP (--▪--), 400 μl of a 50 mg/g suspension of T1144 precipitated by FeSO₄ solution (88%) in 40:60 PLA3L:NMP (--▴--) and 400 μl of a 50 mg/g suspension of T1144 precipitated by FeSO₄ solution (91%) in 40:60 PLA3L:NMP (--∘--).

FIG. 27 shows a plot of T1144 plasma concentrations in cynomolgus monkeys over a 432 hour period postdose for T1144 administered in the following compositions: 800 μl of a 3.5 mg/mL solution of 11144 (-) 400 μl of a 50 mg/g suspension of T1144 precipitated by Zinc solution (89%) in 40:60 PLGA1A:NMP (--♦--) and 400 μl of a 50 mg/g suspension of T1144 precipitated by Zinc solution (60% peptide) 40:60 PLA3L:NMP (--▪--).

FIG. 28 shows a plot of T1144 plasma concentrations in cynomolgus monkeys over a 168 hour period postdose for T1144 administered in the following compositions: 1200 μl of a 3 mg/mL solution of T1144 (-), 400 μl of a suspension of Precipitate H in 40:60 PLA3L:NMP (--□--), 400 μl of a suspension of Precipitate J in 40:60 PLA3L:NMP (--⋄--), 400 μl of a suspension of Precipitate M in 40:60 PLA3L:NMP (--♦--) and 400 μl of a suspension of Precipitate N in 40:60 PLA3L:NMP (--▪--).

FIG. 29 shows the results of an in situ forming gel in a SAIB:PLA:NMP optimization Rat Pharmaceutical (“PK”) Study #526 TRI-1144 concentration in rat plasma. 803-003-A: 100 mg/g TRI-1144 precipitated by zinc sulfate suspended in SAIB:PLA3L:NMP (74:11:15); 803-003-B: 50 mg/g TRI-1144 precipitated by zinc sulfate suspended in SAIB:PLA3L:NMP (65:15:20); 803-003-C: 50 mg/g TRI-1144 precipitated by zinc sulfate suspended in SAIB:PLA3L:NMP (70:15: 15); 803-003-D: 50 mg/g TRI-1144 precipitated by zinc sulfate suspended in SAIB:PLA3L:NMP (75:5:20).

FIG. 30 shows the results of an In Situ Forming Gels in a SAIB:PLA:NMP and PLGA:NMP Comparison Rat PK Study # 526. TRI-1144 concentration in rat plasma. 803-010-A: 50 mg/g TRI-1144 precipitated with excess zinc sulfate suspended in SAIB:PLA3L:NMP (75:5:20); 803-010-E: 50 mg/g TRI-1144 precipitated with zinc sulfate suspended in SAIB:PLA3L:NMP (75:5:20); 803-010-F:50 mg/g TRI-1144 precipitated with excess zinc sulfate suspended in PLA3L:NMP (40:60).

FIG. 31 shows the results of an In Situ Forming Gels PLGA Optimization Monkey PK Study #527. TRI-1144 concentration in monkey plasma. 0782-096: 50 mg/g TRI-1144 precipitated from water by addition of excess zinc sulfate suspended in (50:50)PLA3L:NMP (40:60); 803-020: 50 mg/g TRI-1144 precipitated from water by addition of excess zinc sulfate suspended in SAIB:PLA3L:NMP (75:11:15).

FIG. 32 shows the results of an In Situ Forming Gels PLGA Optimization Rat PK Study #530 and 531. TRI-1144 concentration in rat plasma. 0782-099 50 mg/g TRI-1144 precipitated from 50:50 methanol:water by addition of zinc sulfate suspended in PLA3L:NMP (40:60); 0782-102 50 mg/g TRI-1144 precipitated from water by addition of excess zinc sulfate suspended PLA3L:NMP (40:60).

FIG. 33 shows the results of an In Situ Forming Gels PLGA Optimization Rat PK Study #552. TRI-1144 concentration in rat plasma. 803-050-1 50 mg/g TRI-1144 precipitated from 50:50 methanol:water by addition of zinc sulfate suspended ((50:50)PLGA 2A):NMP (40:60); 803-050-3 50 mg/g TRI-1144 precipitated from 50:50 methanol:water by addition of zinc sulfate suspended ((50:50)PLGA2.5A):NMP (40:60); 803-050-450 mg/g TRI-1144 precipitated from 50:50 methanol:water by addition of zinc sulfate suspended in ((50:50)PLGA 3A):NMP (40:60).

FIG. 34 shows the results of a Study #In Situ Forming Gels PLA-PEG 1500 and PLGA-PEG 1500 Co-polymers:NMPRat PK Study #553. TRI-1144 concentration in rat plasma. 803-051-2: 50 mg/g TRI-1144 precipitated from 50:50 methanol:water by addition of zinc sulfate suspended in ((65:35)PLGA-PEG1500): NMP (40:60); 803-051-3: 50 mg/g TRI-1144 precipitated from 50:50 methanol:water by addition of zinc sulfate suspended in PLA3L-PEGI500:NMP (40:60); 803-051450 mg/g TRI-1144 precipitated from 50:50 methanol:water by addition of zinc sulfate suspended in PLA3L:NMP (40:60).

FIG. 35 shows the a Solvent Ratio and PLA/NMP Optimization PK Study # 560. TRI-1144 concentration in rat plasma. 803-072-8: 50 mg/g TRI-1144 precipitated from 50:50 methanol:water by addition of zinc sulfate suspended in PLA3L:NMP (40:60); 803-072-9: 50 mg/g TRI-1144 precipitated from 50:50 methanol:water by addition of zinc sulfate suspended in PLA3L:NMP (30:70); 803-072-1050 mg/g TRI-1144 precipitated from 50:50 methanol:water by addition of zinc sulfate suspended in (50:50 PLGA3A):NMP (40:60).

FIG. 36 shows the Solvent Optimization PK Study # 561. TRI-1144 concentration in rat plasma 803-073-3: 50 mg/g TRI-1144 precipitated from 50:50 methanol:water by addition of zinc sulfate suspended in (PLA3L:((NMP:PEG400 (50:50))) (30:70); 803-073-4: 50 mg/g TRI-1144 precipitated from 50:50 methanol:water by addition of zinc sulfate suspended in (PLA3L:((NMP:PEG400(50:50))) (40:60); 803-073-5: 50 mg/g TRI-1144 precipitated from 50:50 methanol:water by addition of zinc sulfate suspended in (PLA3L:((NMP:Propylene Glycol (70:30))) (40:60).

FIG. 37 shows the results of a PLGA Site Clearance PK Study #585. TRI-1144 concentration in rat plasma 782-165-1: 50 mg/g TRI-1144 precipitated from 50:50 methanol:water by addition of zinc sulfate suspended in (50:50 PLGA 2.5A:(((NMP:PEG400 (50:50))) (40:60); 782-165-2: 50 mg/g TRI-1144 precipitated from 50:50 methanol:water by addition of zinc sulfate suspended in (50:50 PLGA 2A:((NMP:PEG400 (50:50))) (40:60); 782-165-3: 50 mg/g TRI-1144 precipitated from 50:50 methanol:water by addition of zinc sulfate suspended in (50:50 PLGA IA:((NMP:PEG400(50:50))) (40:60).

### DETAILED DESCRIPTION

### Definitions

The term “patient,” when used herein for purposes of the specification and claims, means a mammal, such as a human. In a particular embodiment, a “patient” is a mammal, such as a human, in need of treatment of a disease or disorder disclosed herein, such as HIV or AIDS.

The term “target cell,” when used herein for purposes of the specification and claims, means a cell capable of being infected by HIV. In one embodiment, the cell is a human cell(s); and in another embodiment, the cell is a human cell(s) capable of being infected by HIV via a process including membrane fusion. In another embodiment, the cell is present in a patient, such as a human patient.

The term “composition,” when used herein for purposes of the specification and claims, means a formulation that comprises a solvent, a gelling material and a bioactive molecule, such as an antiviral peptide. Illustrative compositions are described herein. Compositions provided herein can, for example, be used as medicaments or used to prepare medicaments.

The term “solvent,” when used herein for purposes of the specification and claims, means a water-miscible liquid. In one embodiment, the solvent is a water-miscible liquid and is used in combination with a co-solvent, for example, NMP. A solvent can be used, for example, to dilute the gelling material sufficiently to allow for injection into a patient. Illustrative solvents are described herein and known by those skilled in the art.

The term “gelling material,” when used herein for purposes of the specification and claims, means a solvent-miscible material that, when present in a composition comprising a solvent and a gelling material, forms a matrix upon solvent-subcutaneous fluid exchange, that is, solvent-subcutaneous patient fluid exchange. Illustrative gelling materials are described herein and known by those skilled in the art.

The term “matrix,” when used herein for purposes of the specification and claims, means the biodegradable or bioerodible form that a gelling material takes after solvent-subcutaneous fluid exchange. In one embodiment, the matrix is a viscous (i.e., resistant to shear) matrix. In another embodiment, the matrix is a gel.

The term “vehicle,” when used herein for purposes of the specification and claims, means the liquid material comprising solvent and gelling material that can be employed to deliver a bioactive molecule (e.g., a peptide, such as an antiviral peptide) to a patient. A vehicle can be stored in an aqueous state.

The term “pharmaceutically acceptable carrier,” when used herein for purposes of the specification and claims, means a carrier medium that does not significantly alter the biological activity of the active ingredient (e.g., an HIV fusion inhibitor peptide) to which it is added. A pharmaceutically acceptable carrier includes, but is not limited to, one or more of: water, buffered water, saline, 0.3% glycine, aqueous alcohols, isotonic aqueous solution; and may further include one or more substances such as glycerol, oils, salts, such as sodium, potassium, magnesium and ammonium, phosphonates, carbonate esters, fatty acids, saccharides (e.g., mannitol), polysaccharides, polymers, excipients, and preservatives and/or stabilizers (to increase shelf-life or as necessary and suitable for manufacture and distribution of the composition). In one embodiment, the pharmaceutically acceptable carrier is suitable for intramuscular, subcutaneous or parenteral administration.

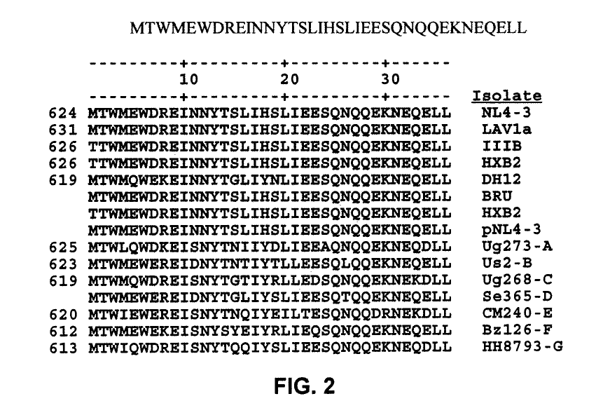

By the term “an amino acid comprising isoleucine or leucine,” unless otherwise specifically pointed out, what is meant for purposes of the specification and claims and in reference to an HIV fusion inhibitor peptide, is to refer to isoleucine or leucine, respectively, or their respective naturally occurring amino acid (e.g., L-amino acid), non-naturally occurring amino acid (e.g., D-amino acid), isomeric form (e.g., norleucine, allo-isoleucine, and the like) or to a derivative form (e.g., tert-leucine). One form of an amino acid isoleucine or leucine can be used to the exclusion of other forms of the amino acid. The HIV fusion inhibitor peptides described herein can also comprise, in their amino acid sequence, one or more polymorphisms found in the sequence of the HR2 region of the HIV gp41 from which each is derived (see, e.g., FIG. 2), except at the one or more positions of the amino acid sequence taught herein to include an amino acid comprising isoleucine or leucine.

The term “HIV” refers to Human Immunodeficiency Virus, and in one embodiment HIV-1.

The term “isolated” when used in reference to a bioactive molecule, e.g., an antiviral peptide such as an HIV fusion inhibitor peptide, or a peptide fragment, means that it is substantially free of components which have not become part of the integral structure of the bioactive molecule itself, e.g., such as substantially free of chemical precursors or other chemicals when chemically synthesized, produced, or modified using biological, biochemical, or chemical processes. In certain embodiments, the isolated bioactive molecule is more than about 75%, 80%, 85%, 90%, 95%, 97%, 99% or 99.9% pure by weight.

The term “between 1 to 3 amino acid substitutions” when used in reference to an HIV fusion inhibitor peptide, means that an HIV fusion inhibitor peptide can also have the amino acid sequence of any one of SEQ ID NOs: 9-15, except that there is not less than one and not more than three amino acid differences compared to any one of SEQ ID NOs: 9-15; while yet still having either (a) more than one leucine zipper-like motif and at least one additional leucine other than a leucine needed to form a leucine zipper-like motif (i.e., other than at position 1 or 8 of a leucine zipper-like motif), or (b) between 3 and 5 leucine zipper-like motifs; and having antiviral activity against HIV (activity in inhibiting HIV-mediated fusion). In that regard, the amino acid differences of an HIV fusion inhibitor peptide having substitutions (when compared to SEQ ID NOs:9-15) are in positions of the amino acid sequence other than the leucine and/or isoleucine residues denoted for HIV fusion inhibitor peptides according to the present invention (see, e.g., illustrations (I) and (II) herein). The not less than one and not more than 3 amino acid differences include, but are not limited to, a conservative amino acid substitution (known in the art to include substitutions of amino acids having substantially the same charge, size, hydrophilicity, and/or aromaticity as the amino acid replaced; examples including, but are not limited to, glycine-alanine-valine, tryptophan-tyrosine, aspartic acid-glutamic acid, arginine-lysine, asparagine-glutamine, and serine-threonine) and/or polymorphisms (e.g., as illustrated in FIG. 2, or as found in laboratory, various clades, and/or clinical isolates of HIV-1). For example, as related to SEQ ID NOs: 11, 12, or 13, an HIV fusion inhibitor peptide has between one to 3 amino acid differences that are in positions other than amino acid residues 10, 17, 24, 31, and 38 of any one of SEQ ID NOs: 11, 12, or 13. For example, as related to SEQ ID NO:9 and SEQ ID NO:14, an HIV fusion inhibitor peptide has between one to 3 amino acid differences that are in positions other than amino acid residues 10, 17, 21, 24, and 38 of SEQ ID NO:9 or of SEQ ID NO:14. For example, as related to SEQ ID NO:10 or SEQ ID NO:15, an HIV fusion inhibitor peptide has between one to 3 amino acid differences that are in positions other than amino acid residues 10, 17, 21, 31, and 38 of SEQ ID NO:10 or SEQ ID NO:15. An illustrative example of this embodiment includes, but is not limited to, an amino acid sequence of SEQ ID NO:16, wherein a position that may be the site of an amino acid difference of the between one and three amino acid substitutions is denoted by Xaa (representing any amino acid, naturally or non-naturally occurring; i.e., more than one possible amino acid may be used in this amino acid position). Also, one or more conservative amino acid substitutions can be made, such as a lysine substituted by an arginine or histidine, an arginine substituted by a lysine or histidine, a glutamic acid substituted by an aspartic acid, or an aspartic acid substituted by a glutamic acid. Amino acid positions 10, 17, 21, 24, 31, and 38 are underlined for illustrative purposes. In also referring to SEQ ID NOs:9-15, note that in SEQ ID NO:16 “Zaa” is used to denote an amino acid that may be either leucine or isoleucine; and Baa is used to denote an amino acid that is preferably either leucine, isoleucine, but may be Xaa, except that at least one Baa is either a leucine or isoleucine.

SEQ ID NO: 16:
XaaXaaXaaEAXaaDRAZaaAEXaaAARZaaEAZaaZaaRABaaXaaE
XaaXaaEKBaaEAAZaaREZaa

The HIV fusion inhibitor peptides described herein can also, for example, include peptides derived from the HR2 region of HIV gp41 corresponding to SEQ ID NO:5 (by sequence alignment) present in laboratory, clades or clinical isolates of HIV-1, for example, those laboratory strains and clinical isolates listed in FIG. 2, as long as the HIV fusion inhibitor peptides satisfy the amino acid requirements of SEQ ID NO:16. In one embodiment, such HIV fusion inhibitor peptides exhibit from between 1 to 3 amino acid substitutions, compared to any of SEQ ID NOs:9-15. In one embodiment, the HIV fusion inhibitor peptide further comprises a N-terminal blocking group or C-terminal blocking group, or both; and those terminal groups may include, but are not limited to: an amino group or an acetyl group at the N-terminus; and a carboxyl group or an amido group at the C-terminus.

The HIV fusion inhibitor peptides described herein can also include peptides exhibiting the variant amino acid sequences of any of the peptides disclosed in US 2006/0247416, the entire contents of which is incorporated herein by reference in its entirety, as long as the HIV fusion inhibitor peptides satisfy the amino acid requirements of SEQ ID NO:16. In one embodiment, such HIV fusion inhibitor peptides exhibit from between 1 to 3 amino acid substitutions compared to any one of SEQ ID NOs:9-15. In a preferred embodiment, the HIV fusion inhibitor peptide is between 14 and 60 amino acid residues in length. In one embodiment, the HIV fusion inhibitor peptide further comprises a N-terminal blocking group or C-terminal blocking group, or both; and those terminal groups may include, but are not limited to: an amino group or an acetyl group at the N-terminus; and a carboxyl group or an amido group at the C-terminus.

The term “reactive functionality,” when used herein for purposes of the specification and claims, means a chemical group or chemical moiety that is capable of forming a bond with another chemical group or chemical moiety. With respect to chemical groups, a reactive functionality is known to those skilled in the art to comprise a group that includes, but is not limited to, maleimide, thiol, carboxylic acid, hydrogen, phosphoryl, acyl, hydroxyl, acetyl, hydrophobic, amine, amido, dansyl, sulfa, a succinimide, a thiol-reactive, an amine-reactive, a carboxyl-reactive, and the like. One reactive functionality can be used to the exclusion of another reactive functionality.

The term “linker,” when used herein for purposes of the specification and claims, means a compound or moiety that acts as a molecular bridge to operably link two different molecules (e.g., a first reactive functionality of a linker is covalently coupled to a reactive functionality of a macromolecular carrier, and a second reactive functionality of the linker is covalently coupled to a reactive functionality of an HIV fusion inhibitor peptide). The linker can be amino acids, as in production of a recombinant fusion protein containing one or more copies of the HIV fusion inhibitor peptide. Alternatively, the two different molecules can be linked to the linker in a step-wise manner (e.g., via chemical coupling). In general, there is no particular size or content limitations for the linker so long as it can fulfill its purpose as a molecular bridge. Linkers are known to those skilled in the art to include, but are not limited to, chemical chains, chemical compounds (e.g., reagents), amino acids, and the like. The linkers can include, but are not limited to, homobifunctional linkers, heterobifunctional linkers, biostable linkers, hydrolysable linkers, and biodegradable linkers, as well as others known in the art. Heterobifunctional linkers, well known to those skilled in the art, contain one end having a first reactive functionality to specifically link a first molecule, and an opposite end having a second reactive functionality to specifically link to a second molecule. It will be evident to those skilled in the art that a variety of monofunctional, difunctional, and polyfunctional reagents (such as those described in the catalog of the Pierce Chemical Co., Rockford, Ill.) can be employed as a linker. Depending on such factors as the molecules to be linked, the conditions in which the linking is performed, and the intended pharmacokinetic properties upon administration, the linker can vary in length and make-up for optimizing such properties as: preservation of biological activity and function, stability, resistance to certain chemical and/or temperature parameters, susceptibility to cleavage in vivo, and of sufficient stereo-selectivity or size.

The term “macromolecular carrier” when used herein for purposes of the specification and claims, means a molecule which is linked, joined, or fused (e.g., chemically, or through recombinant means using genetic expression) to one or more peptides according to the present invention, whereby the molecule is capable of conferring one or more properties of: stability to the one or more peptides, increase in biological activity of the one or more peptides, or an increase in plasma half-life of the one or more peptides (e.g., prolonging the persistence of the one or more peptides in the body) relative to that with respect to the one or more peptides in the absence of the molecule. Such macromolecular carriers are well known in the art to include, but are not limited to, serum proteins, polymers, carbohydrates, and lipid-fatty acid conjugates. Serum proteins typically used as macromolecular carriers include, but are not limited to, transferring, albumin (preferably human), immunoglobulins (preferably human IgG or one or more chains thereof), or hormones. Polymers typically used as macromolecular carriers include, but are not limited to, polylysines or poly(D-L-alanine)-poly(L-lysine)s, or polyols. A polyol can comprise a water-soluble poly(alkylene oxide) polymer, and can have a linear or branched chain(s). A polymer can be a branched chain polyol (such as a PEG, having multiple (for example, 3 or more) chains, each which can be coupled to the HIV fusion inhibitor peptide directly or via a linker); and in one embodiment, a branched chain polyol that is biodegradable, and/or cleaved over time, under in vivo conditions. Suitable polyols include, but are not limited to, polyethylene glycol (PEG), polypropylene glycol (PPG), and PEG-PPG copolymers. In one embodiment, a polyol comprises PEG having an average molecular size selected from the range of from about 1,000 Daltons to about 20,000 Daltons. Other types of macromolecular carriers that can be used, which generally have molecular weights higher than 20,000 Daltons, are known in the art.

The term “chemical protecting group,” or “CPG,” when used herein for purposes of the specification and claims, means a chemical moiety that is used to block a reactive functionality comprising an amine group from chemically reacting with another reactive functionality. Chemical protecting groups are well known by those in the art of peptide synthesis to include, but are not limited to, Dmcp (dimethylcyclopropylmethyl), Bsmoc (Benzo[b]thiophenesulfone-2-methoxycarbonyl), tBu (t-butyl), trt (triphenylmethyl(trityl)), OtBu (tert-butoxy), Boc or t-Boc (tert-butyloxycarbonyl), Fmoc (9-fluorenylmethoxycarbonyl), Aoc (t-amyloxy-carbonyl), TEOC (β-trimethylethyloxycarbonyl), CLIMOC (2-chloro-1-indanyl methoxyl carbonyl), BIMOC (benz-[f]indene-3-methoxylcarbonyl), PBF (2,2,4,6,7-pentamethyldihydrobenzofuan-5-sulfonyl), 2-Cl-Z (chlorobenzyl-oxycarbonyl), Alloc (allyloxycarbonyl), Cbz (benzyloxycarbonyl), Adoc (adamantyloxy-carbonyl), Mcb (1-methylcyclobutyloxycarbonyl), Bpoc (2-(p-biphenylyl) propyl-2-oxycarbonyl), Azoc (2-(p-phenylazophenyl) propyl-2-oxycarbonyl), Ddz (2,2 dimethyl-3,5-dimethyloxybenzyl-oxycarbonyl), MTf (4-methoxy-2,3,6-trimethoylbenzenesulfonyl), PMC (2,2,5,7,8-pentamethylchroman-6-sulfonyl), Tos (tosyl), Hmb (2-hydroxyl-4-methoxybenzyl), Poc (2-phenylpropyl-2-oxycarbonyl), Dde (1-(4,4-dimethyl-2,6-dioxocyclohex-1-ylidene)ethyl), ivDde (1-(4,4-dimethyl-2,6-dioxo-cyclohex-1-ylidene)-3-methylbutyl), benzyl, dansyl, para-nitrobenzyl ester, and the like. One chemical protecting group can be used to the exclusion of another chemical protecting group.

The term “deprotection,” when used herein for purposes of the specification and claims, is known in the art to mean a process by which one or more chemical protecting group(s) is removed from a molecule containing one or more chemical protecting groups, wherein the molecule comprises an amino acid, peptide fragment, or HIV fusion inhibitor peptide. Generally, the deprotection process involves reacting the molecule protected by one or more chemical protecting groups with a chemical agent that removes the chemical protecting group. For example, an N-terminal alpha amino group, which is protected by a chemical protecting group, can be reacted with a base to remove base labile chemical protecting groups (e.g., Fmoc, and the like). Chemical protecting groups (e.g., Boc, TEOC, Aoc, Adoc, Bopc, Ddz, Cbz, and the like) are removed by acid. Other chemical protecting groups, particularly those derived from carboxylic acids, can be removed by acid or a base.

The term “derivative(s)” when used herein for purposes of the specification and claims, means a compound that arises from a parent compound by replacement of one or more atoms with another atom or group of atoms which, in the case of an antiviral compound, preferably retains all or substantially all of the antiviral activity of the parent compound.

The terms “first,” “second,” “third,” and the like, may be used herein to: (a) indicate an order; or (b) to distinguish between molecules or reactive functionalities of a molecule; or (c) a combination of (a) and (b). However, the terms “first,” “second,” “third,” and the like, are not otherwise to be construed as limiting the invention.

The terms “peptide fragment” and “intermediate” are used synonymously herein, in relation to an HIV fusion inhibitor peptide according to the present invention, and for the purposes of the specification and claims, to mean a peptide comprising an amino acid sequence of no less than about 5 amino acids and no more than about 30 amino acid residues in length, and comprises at least a portion (as contiguous amino acids) of the amino acid sequence of that HIV fusion inhibitor peptide. See Examples 4-7, and Tables 4, 5, 7 & 8 herein, for illustrative examples of peptide fragments useful for making SEQ ID NOs: 9 and 10. Further, while in a preferred embodiment peptide fragments (singly or when combined as a group to form an HIV fusion inhibitor peptide) are synthesized such that peptidic bonds are formed between the amino acid residues, it is readily apparent to one skilled in the art that non-peptidic bonds may be formed using reactions known to those skilled in the art (e.g., imino, ester, hydrazide, azo, semicarbazide, and the like).

The term “pharmacokinetic properties,” when used herein for purposes of the specification and claims, means the total amount of bioactive molecule (e.g., antiviral peptide) in a composition that is systematically available over time. Pharmacokinetic properties can be determined by measuring total systemic concentrations of the bioactive molecule (e.g., antiviral peptide) over time after being administered in vivo. As an example, pharmacokinetic properties can be expressed in terms of the Area Under the Curve (AUC), biological half-life, and/or clearance. AUC is the integrated measure of systemic bioactive molecule concentrations over time in units of mass-time/volume. Following the administration of a dose of bioactive molecule, the AUC from the time of dosing to the time when no active ingredient remains in the body, is a measure of the exposure of the individual to the bioactive molecule (and/or a metabolite of the bioactive molecule). A composition has “improved” or “increased” pharmacokinetic properties when the bioactive molecule(s) which it contains has one or more of: (a) a longer (“increase”) in biological (terminal elimination) half life (“t ½), (b) a reduction in biological (total body) clearance (Cl), (c) a longer sustained release profile, (d) an increased weight percent (e.g., about 10% or more) incorporation into the composition, (e) a decreased or lower Cₘₐₓ, (f) a longer tₘₐₓ, and (g) a longer t_{0.01} or t_{0.1}, as compared to that of the bioactive molecule(s) contained in a formulation other than those described herein. In one embodiment, improved pharmacokinetics means a clearance of a bioactive molecule that is reduced, relative to that of a compared formulation, such as typically being from about 2 fold reduction to about 10 fold reduction. In another embodiment, improved pharmacokinetics means an increase in biological half-life of from about a 10% increase to about a 60% increase relative to that of a formulation subjected to comparison. Improved pharmacokinetics can also encompass both a reduction in clearance and an increase in biological half-life. In one embodiment, desirable pharmacokinetic properties include reach Cₘₐₓ quickly (e.g., within 8, 12, 16, 20, 24, 28, 32, 36 or 48 hours) followed by relatively constant plasma concentrations for 5, 7, 10, 14, 17, 21 or 28 days or longer. In a particular embodiment, desirable pharmacokinetic properties for the compositions provided herein are a lower Cₘₐₓ, a longer tₘₐₓ and a longer t_{0.01} or t_{0.1}. The equations used to calculate area-under the plasma concentration vs. time curve (AUC), total body clearance (Cl), and terminal elimination half-life (t ½) are set forth herein in Example 1.

The term “in solution,” as standard in the art in referring to an aqueous fluid into which is dissolved one or more solids, is used herein for the purposes of the specification and claims to mean an aqueous solution containing a bioactive molecule such as an HIV fusion inhibitor peptide dissolved therein under realistic use conditions of concentration and temperature as described herein in more detail and as standard in the art for an injectable drug formulation. There are various ways known in the art to distinguish formation of a solution, as opposed to formation of a suspension, such as checking for visual clarity (transparency of a solution versus cloudiness of a suspension), light transmission, and the like. “Solubility” is determined by the amount (e.g., weight percent) of bioactive molecule such as an HIV fusion inhibitor peptide that is present in solution in an aqueous fluid without showing observed evidence of precipitation out of solution, or gelling of the aqueous fluid containing the bioactive molecule.

The term “sustained-release,” when used herein for purposes of the specification and claims, means that upon administration a bioactive molecule is released continuously over specified time intervals.

The term “effective amount,” when used herein for purposes of the specification and claims, means an amount of a bioactive molecule that will achieve the desired result of a particular method. In one embodiment, an effective amount of a biomolecule can be an amount that is sufficient (by itself and/or in conjunction with a regimen of doses) to reduce (e.g., relative to that in the absence of the bioactive molecule) HIV viral load in a patient. In another embodiment, an effective amount of a biomolecule can be an amount sufficient to inhibit (e.g., relative to that in the absence of the bioactive molecule) infection of a cell by HIV or to inhibit viral entry of a target cell. Such inhibition can be measured using assays known in the art. In another embodiment, an effective amount of a biomolecule can be an amount sufficient to ameliorate a symptom associated with an HIV infection. An effective amount of a biomolecule can be determined by one skilled in the art using data from routine in vitro and in vivo studies well know to those skilled in the art.

The terms “treatment” or “therapy,” or grammatical equivalents thereof, are used interchangeably with respect to HIV infection, and for purposes of the specification and claims, mean that a composition comprising a bioactive molecule such as an HIV fusion inhibitor peptide can be used to affect one or more processes associated with HIV infection, or one or more parameters or endpoints used as indicators for determining the therapeutic effect of such treatment or therapy (e.g., “therapeutic application”). For example, the bioactive molecule can be used to inhibit one or more of the following processes: transmission of HIV to a target cell; fusion between HIV and a target cell (“HIV fusion”); viral entry (the process of HIV or its genetic material entering into a target cell during the infection process); and syncytia formation (e.g., fusion between an HIV-infected cell, and a target cell). Viral suppression (determined by methods known in the art for measuring the viral load of HIV in a body fluid or tissue) is a commonly used primary endpoint, and an increase in the number of CD4⁺ cells circulating in the bloodstream is a commonly used secondary endpoint, for assessing the efficacy of a drug in treatment or therapy of HIV infection; each being a measurable effect of inhibiting transmission of HIV to a target cell. Thus, a composition comprising a bioactive molecule such as an HIV fusion inhibitor peptide can be used to affect a therapeutic application comprising viral suppression and/or an increase in the relative number of circulating CD4⁺ cells.

### Compositions

Provided herein are compositions useful for administering a bioactive molecule(s) to a patient. Specifically, provided herein are compositions comprising a solvent, a gelling material and a bioactive molecule, such as an antiviral peptide. Without being limited by theory, the embodiments provided herein are based, at least in part, on the unexpected discovery that an increased weight percent of a bioactive molecule can be incorporated in the compositions while exhibiting a desirable sustained release profile upon administration to a patient. The compositions described herein can constitute in situ forming gel compositions in that the compositions come to comprise a matrix when administered to a patient and solvent-subcutaneous fluid exchange occurs.

In one embodiment, upon administration to a patient, the compositions provided herein yield plasma concentrations of a biomolecule that quickly (e.g., within 8, 12, 16, 20, 24, 28, 32, 36 or 48 hours) reach Cₘₐₓ and then provide relatively constant plasma concentrations of the biomolecule for 5, 7, 10, 14, 17, 21 or 28 days or longer. In a particular embodiment, desirable pharmacokinetic properties for the compositions provided herein are a lower Cₘₐₓ, a longer tₘₐₓ and a longer t_{0.01} or t_{0.1}.

The compositions provided herein are, for example, useful for administering compositions comprising certain antiviral peptides, referred to as T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9), or a combination of two or more of these peptides, as well as derivatives of the T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9) peptides.

In one embodiment, the compositions comprise a solvent, a gelling material that forms a matrix upon solvent-subcutaneous fluid exchange, and at least one bioactive molecule, e.g., an antiviral peptide such as T20 (SEQ ID NO:2), T1249 (SEQ ID NO:57), T897 (SEQ ID NO:58), T2635 (SEQ ID NO:5), T999 (SEQ ID NO:59) and T1144 (SEQ ID NO:9) or a derivative thereof. The compositions can, for example, exist as a solution or suspension prior to administration. The solution or suspension can be aqueous or contain organic solvents. In one embodiment, the compositions can be stored at room temperature for up to 18 months. Upon administration and exposure to fluid in the subcutaneous space of a patient, the gelling material can form a matrix which is biodegradable or at least bioerodible. Thus, in one embodiment, the compositions can be administered in liquid form, e.g., by subcutaneous injection, to a patient in need thereof. The resulting matrix can, for example, act as a sustained-release matrix for the bioactive molecule(s).

The compositions provided herein generally comprise at least one gelling material in a sufficient amount (e.g., about 50-1000 mg/g, 100-900 mg/g, 150-900 mg/g, 200-900 mg/g, 250-900 mg/g, 500-900 mg/g, 750-900 mg/g or 750-1000 mg/g) to form a matrix upon administration to a patient. In one embodiment, the appropriate amount (in mg/g) of gelling material can be determined by adding vehicle gelling material compositions from the vehicle ratio and multiplying by 10. In one embodiment, the gelling material is a lactide or glycolide polymer. In a particular embodiment, the gelling material is sucrose acetate isobutyrate (SAIB), polylactide (PLA, e.g., PLA3L, PLA3M, or PLA-PEG) or polylactide-co-glycolide (PLG, PLGA, e.g., PLGA1, PLGA-glucose, or PLGA-PEG). The compositions provided herein can also comprise a bioactive molecule, such as an antiviral peptide. In one embodiment, the compositions provided herein comprise a sufficient concentration of the bioactive molecule such that an effective dose of the bioactive molecule is released, e.g., in a sustained release manner, from a matrix formed upon administration to a patient.

The compositions provided herein can generally comprise and be administered with concentrations of a bioactive molecule of at least 5%. Accordingly, in one embodiment, compositions provided herein comprise an amount of bioactive molecule (e.g., antiviral peptide) equal to or about 5%, 6%, 7%, 8%, 9%, 10%, 11%, 12%, 13%, 14%, 15%, 20% or more by weight of the composition. In one embodiment, compositions provided herein comprise about 80-90 mg/ml, 90-100 mg/ml, 100-105 mg/ml, 105-110 mg/ml, 110-125 mg/ml, 125-130 mg/ml, 130-140 mg/ml, 140-150 mg/ml, 150-200 mg/ml, 200-250 mg/ml, 250-300 mg/ml of bioactive molecule.

In a particular embodiment, upon administration to a patient, compositions provided herein form a matrix that provides an initial burst of the bioactive molecule, followed by sustained release of the bioactive molecule for at least 5, 7, 10 or 14 days after administration of the composition. In certain embodiments, the initial burst of bioactive molecule provided by administering a composition provided herein to a patient is a Cₘₐₓ of at least or about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml or 15 μg/ml or more within 2, 4, 6, 8, 10, 12, 14, 16, 18, 20 or 24 hours after administration of the composition. In certain embodiments, the sustained release of the bioactive molecule provided by administering a composition provided herein to a patient is greater than or equal to 0.1 μg/ml, 0.5 μg/ml, 1 μg/ml, 1.25 μg/ml, 1.5 μg/ml or 2.0 μg/ml or more for at least 5, 7, 10, 14, 21, 25 or 28 days or longer after administration of the composition.

In another embodiment, provided herein are compositions that, upon administration to a patient, form a matrix (e.g., in situ) and provide a Cₘₐₓ of a bioactive molecule (e.g., an antiviral peptide) of at least 10 μg/ml within 12 hours of administration followed by sustained release that results in plasma levels of at least 1 μg/ml for at least 7 days.

In one embodiment, compositions comprising an antiviral peptide provided herein further comprise at least one additional component such as a pharmaceutically acceptable carrier, a macromolecule, or a combination thereof.

In another embodiment, the compositions comprise one or more additional bioactive molecules. In one embodiment, the compositions can comprise a T20, T1249, T897, T2635, T999 or T1144 peptide or a derivative thereof. In another embodiment, such compositions can comprise or further comprise one or more other antiviral agents. Other antiviral agents which can be used in the compositions, either by themselves or as part of a combinatorial therapy regime, include but are not limited to DP107 (T21) or any other antiviral polypeptide, such as those described in U.S. Pat. No. 6,541,020 B1, incorporated herein by reference in its entirety. Other exemplary, non-limiting examples of therapeutic agents include antiviral agents such as cytokines, e.g., rIFN α, rIFN β, rIFN γ; reverse transcriptase inhibitors, including but not limited to, abacavir, AZT (zidovudine), ddC (zalcitabine), nevirapine, ddI (didanosine), FTC (emtricitabine), (+) and (−) FTC, reverset, 3TC (lamivudine), GS 840, GW-1592, GW-8248, GW-5634, HBY097, delaviridine, efavirenz, d4T (stavudine), FLT, TMC125, adefovir, tenofovir, and alovudine; protease inhibitors, including but not limited to, amprenivir, CGP-73547, CGP-61755, DMP-450, indinavir, nelfinavir, PNU-140690, ritonavir, saquinavir, telinavir, tipranovir, atazanavir, lopinavir, ABT378, ABT538 and MK639; inhibitors of viral mRNA capping, such as ribavirin; amphotericin B as a lipid-binding molecule with anti-HIV activity; castanospermine as an inhibitor of glycoprotein processing; viral entry inhibitors such as fusion inhibitors (enfuvirtide, T1249, other fusion inhibitor peptides, and small molecules), SCH-D (Schering-Plough), UK-427857 (Pfizer), TNX-355 (Tanox Inc.), AMD-070 (AnorMED), Pro 140, Pro 542 (Progenics), FP-21399 (EMD Lexigen), BMS806, BMS-488043 (Bristol-Myers Squibb), maraviroc (UK-427857), ONO-4128, GW-873140, AMD-887, CMPD-167, and GSK-873,140 (GlaxoSmithKline); CXCR4 antagonist, such as AMD-070); lipid and/or cholesterol interaction modulators, such as procaine hydrochloride (SP-01 and SP-01A); integrase inhibitors, including but not limited to, L-870, and 810; RNAseH inhibitors; inhibitors of rev or REV; inhibitors of vif (e.g., vif-derived proline-enriched peptide, HIV-1 protease N-terminal-derived peptide); viral processing inhibitors, including but not limited to betulin, and dihydrobetulin derivatives (e.g., PA-457); and immunomodulators, including but not limited to, AS-101, granulocyte macrophage colony stimulating factor, IL-2, valproic acid, and thymopentin.

In one embodiment, provided herein are compositions wherein the bioactive molecule (e.g., antiviral peptide) is dissolved.

In another embodiment, provided herein are compositions wherein the bioactive molecule (e.g., antiviral peptide) is suspended.

In another embodiment, provided herein are compositions wherein the suspended bioactive molecule (e.g., antiviral peptide) is in a spray-dried form.

In another embodiment, provided herein are compositions wherein the suspended bioactive molecule (e.g., antiviral peptide) is in a spray-dried form comprising a salt (e.g., a metal salt).

In another embodiment, provided herein are compositions wherein the suspended bioactive molecule (e.g., antiviral peptide) is in a spray-dried form comprising zinc.

In another embodiment, provided herein are compositions wherein the suspended bioactive molecule (e.g., antiviral peptide) is in a spray-dried form comprising calcium.

In another embodiment, provided herein are compositions wherein the suspended bioactive molecule (e.g., antiviral peptide) is in a spray-dried form comprising iron.

In another embodiment, provided herein are compositions wherein the suspended bioactive molecule (e.g., antiviral peptide) is in a precipitated form.

In another embodiment, provided herein are compositions wherein the suspended bioactive molecule (e.g., antiviral peptide) is in a precipitated form comprising a salt (e.g., a metal salt).

In another embodiment, provided herein are compositions wherein the suspended bioactive molecule (e.g., antiviral peptide) is in a precipitated form comprising zinc.

In another embodiment, provided herein are compositions wherein the suspended bioactive molecule (e.g., antiviral peptide) is in a precipitated form comprising calcium.

In another embodiment, provided herein are compositions wherein the suspended bioactive molecule (e.g., antiviral peptide) is in a precipitated form comprising iron.

In another embodiment, provided herein are compositions wherein the suspended bioactive molecule (e.g., antiviral peptide) is in a precipitated form comprising magnesium.

In another embodiment, provided herein are compositions wherein the suspended bioactive molecule (e.g., antiviral peptide) is in a precipitated form comprising copper.

In another embodiment, provided herein are compositions wherein the suspended bioactive molecule (e.g., antiviral peptide) is in a precipitated form comprising aluminum.

In another embodiment, provided herein are compositions comprising about 1-15%, 1-14%, 1-13%, 1-12%, 1-11%, 1-10%, 1-9%, 1-8%, 1-7%, 1-6%, 1-5%, 1-4%, 1-3%, 1-2%, 5-15%, 7-15%, 5-10%, 7-10% or 10-15% of a salt, such as a metal salt. In a further embodiment, provided herein are compositions comprising a salt, such as a metal salt, present in a 1:1 molar ratio to a biomolecule, such as an antiviral peptide. Particular examples of metal salts useful in the compositions provided herein include, but are not limited to, zinc, calcium and iron.

Altering the ratio of gelling materials in a composition provided herein can modulate, e.g., improve or optimize, the delivery rate of a biomolecule from a matrix formed upon administration of a composition provided herein to a patient. In a particular embodiment, decreasing the SAIB:PLA ratio in a composition provided herein can result in an increase in the plasma concentration of a biomolecule at any particular time in a patient and can increase the duration in which a particular plasma level of a biomolecule is maintained in a patient.

In a particular embodiment, using NMP as the solvent in place of triacetin or benzylbenzoate in a composition provided herein can result in an increase in the plasma concentration of a biomolecule at any particular time in a patient and can increase the duration in which a particular plasma level of a biomolecule is maintained in a patient. In a specific embodiment, using NMP as the solvent in place of triacetin can result in an increase in Cₘₐₓ.

In another embodiment, increasing the administered injection volume (e.g., doubling) of a composition provided herein can result in an increase in the plasma concentration of a biomolecule at any particular time in a patient and can increase the duration in which a particular plasma level of a biomolecule is maintained in a patient.

In another embodiment, the type of gelling material (e.g., PLA) used can affect the pharmacokinetic parameters of compositions provided herein. In a particular embodiment, changing the PLA type from 3L to 3M can result in a decrease in Cₘₐₓ, an increase in tₘₐₓ and an increase in t_{0.01}.

In another embodiment, altering the ratio of gelling material to solvent in a composition provided herein can influence the delivery rate of a biomolecule. In a particular embodiment, increasing gelling material to solvent ratio (e.g., PLGA1A to NMP) in a composition provided herein can result in a decrease in Cₘₐₓ, an increase in tₘₐₓ and an increase in t_{0.01}.

In another embodiment, altering the polymer type and molecular weight can improve the delivery rate of a biomolecule. In a particular embodiment, increasing polymer molecular weight and/or increasing L:G (lactide:glycolide) ratio can result in a decrease in Cₘₐₓ, an increase in tₘₐₓ and an increase in t_{0.01}.

In another embodiment, increasing the peptide concentration (e.g., doubling) in a composition provided herein can result in a decrease in Cₘₐₓ, an increase in tₘₐₓ and a decrease in t_{0.01}.

In another embodiment, increasing the amount of gelling material (e.g., PLGA) in the vehicle can result in a decrease in Cₘₐₓ, an increase in tₘₐₓ and a decrease in t_{0.01}.

In another embodiment, increasing the amount of gelling material (e.g., PLA) in the vehicle (e.g., an SAIB containing vehicle) slows the release (e.g., decreases Cₘₐₓ, increases tₘₐₓ and/or increases t_{0.01}) of a biomolelcule from compositions provided herein. In a particular embodiment, increasing the amount of PLA in the vehicle from 1% to 5% further slows the release of a biomolecule from compositions provided herein. In another embodiment, increasing the amount of PLA in the vehicle from 5% to 10% further slows the release of a biomolecule from compositions provided herein.

### Solvents

Solvents which are useful in the compositions and methods provided herein include any water-miscible liquid that can dilute the gelling material sufficiently to allow for injection of the composition into a patient. In one embodiment, the solvent is N-methyl-2-pyrrolidone (NMP). Other suitable solvents include, but are not limited to, water, alcohols (e.g., methyl, ethyl, isopropyl and benzyl alcohol), glycols (e.g., polyethylene, propylene and tetra glycol), benzoates (e.g., ethylbenzoate and benzylbenzoate), glycerides (e.g., mono-, di- and tri-glycerides), triacetin and pharmaceutically acceptable esters (e.g., ethyl-lactate and propyl-carbonate).

### Gelling Materials

Gelling materials which are useful in the compositions and methods provided herein include any solvent-miscible material that forms a matrix upon solvent-subcutaneous fluid exchange. In one embodiment, the gelling material is sucrose acetate isobutyrate (SAIB) or a derivative thereof, for example, sucrose acetate or sucrose acetate isobutyrate-special grade (SAIB-SG). In another embodiment, the gelling material is polylactide (PLA), for example, PLA3L or PLA3M. In another embodiment, the gelling material is polylactide-co-glycolide (PLG, PLGA, PLGA-glucose or a derivative thereof, for example, PLGA-PEG1500 or PLA-PEG1500). In another embodiment, the gelling material is poly-caprolactone or a derivative or lactide/glycolide co-polymer thereof. PLA and PLGA differ in lactide:glycolide ratio, molecular weight and their endgroup. Molecular weight is graded by the number in the name. An estimate of the molecular weight is 10000 times the number. The endgroup is either carboxylic acid (A), methyl ester (M) or lauryl ester (L).

In one embodiment, the compositions provided herein can contain two or more different gelling agents present at the same or different weight percents. In a particular embodiment, the gelling material is a mixture of two or more materials selected from PLA, PLG, PLGA or PLGA-glucose.

In certain embodiments, the gelling material is present in an amount between about 5-95% by weight, about 5-90% by weight, about 10-90% by weight, about 10-85% by weight, about 15-85% by weight, about 20-85% by weight, about 30-85% by weight, about 30-80% by weight, about 30-70% by weight, about 30-65% by weight, about 30-60% by weight, about 40-85% by weight, about 45-85% by weight, about 50-85% by weight, about 55-85% by weight, about 60-85% by weight, about 65-85% by weight, about 70-85% by weight, about 75-85% by weight, about 80-85% by weight, about 1-15% by weight, about 5-15% by weight or about 10-15% by weight.

In another embodiment, the gelling material is present at an amount of about 25-900 mg/g, 100-900 mg/g, 200-900 mg/g, 300-900 mg/g, 400-900 mg/g, 500-900 mg/g, 100-800 mg/g, 100-700 mg/g, 100-600 mg/g, 100-500 mg/g, 200-800 mg/g, 300-600 mg/g, 25-250 mg/g, 25-200 mg/g, 25-150 mg/g, 50-150 mg/g, 50-100 mg/g, 50 mg/g, 75 mg/g or 100 mg/g.

### Peptides

With respect to bioactive molecules that are antiviral peptides, any antiviral peptide known in the art can be used in the compositions and methods provided herein. In one embodiment, the antiviral peptide is a T20, T1249, T897, T2635, T999 or T1144 peptide or a derivative thereof.

Particular antiviral peptides useful in the compositions and methods provided herein include HIV fusion inhibitor peptides derived from a base amino acid sequence (“base sequence”) having an amino acid sequence of SEQ ID NO:5, but wherein each HIV fusion inhibitor peptide differs from the base sequence by having more than one leucine zipper-like motif in its amino acid sequence, and having at least one additional leucine present in its amino acid sequence other than that necessary to form a leucine zipper-like motif (i.e., an amino acid in the sequence other than at amino acid position 1 or 8 of a leucine zipper-like motif; as exemplified by substituting isoleucine by leucine at amino acid position 21 of SEQ ID NO:5).

Further antiviral peptides useful in the compositions and methods provided herein include HIV fusion inhibitor peptides derived from a base amino acid sequence (“base sequence”) having an amino acid sequence of SEQ ID NO:5, but wherein each HIV fusion inhibitor peptide differs from the base sequence by having more than two leucine zipper-like motif in its amino acid sequence.

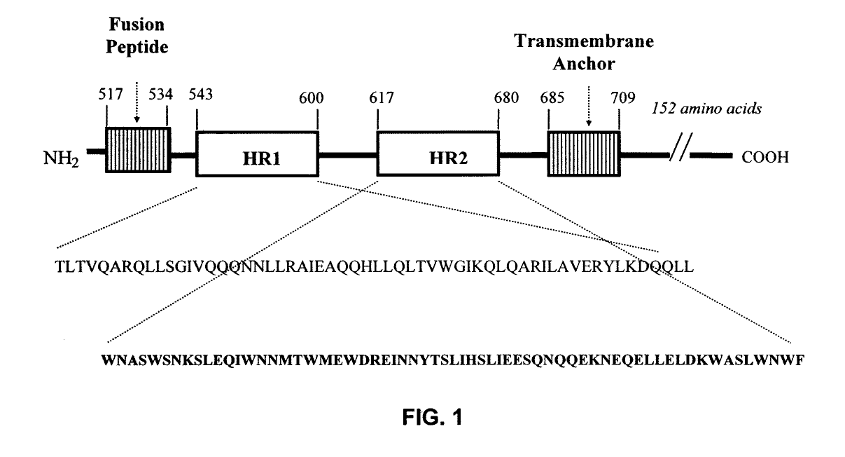

Further antiviral peptides useful in the compositions and methods provided herein include a series of HIV fusion inhibitor peptides, wherein each HIV fusion inhibitor peptide: (a) contains amino acid sequence derived from the HR2 region of HIV gp41; (b) has an amino acid sequence having not less than 2 and not more than 5 leucine zipper-like motifs; (c) having at least one additional leucine (e.g., compared to a base sequence of any one or more of SEQ ID NOs: 5-7) in its amino acid sequence other than at amino acid position 1 or 8 of a leucine zipper-like motif; and optionally (d) demonstrates an unexpected improvement in one or more biological properties. In one embodiment, the HIV fusion inhibitor peptide contains an amino acid sequence derived from the HR2 region of HIV gp41, wherein the amino acid sequence comprises the HR2 leucine zipper-like motif, e.g., the HR2 leucine zipper-like motifs depicted in FIG. 1 or FIG. 2. In a preferred embodiment, the HIV fusion inhibitor peptide is between 14 and 60 amino acid residues in length. In one embodiment, the HIV fusion inhibitor peptide further comprises a N-terminal blocking group or C-terminal blocking group, or both; those terminal blocking groups may include, but are not limited to: an amino group or an acetyl group at the N-terminus; and a carboxyl group or an amido group at the C-terminus.

Further antiviral peptides useful in the compositions and methods provided herein include HIV fusion inhibitor peptides, wherein each HIV fusion inhibitor peptide: (a) contains amino acid sequence derived from the HR2 region of HIV gp41; (b) has an amino acid sequence having greater than 2 and not more than 5 leucine zipper-like motifs; and (c) having at least one additional leucine (e.g., compared to a base sequence of any one or more of SEQ ID NOs: 5-7) in its amino acid sequence other than at amino acid position 1 or 8 of a leucine zipper-like motif; and d) demonstrates an unexpected improvement in one or more biological properties. In one embodiment, the HIV fusion inhibitor peptide contains an amino acid sequence derived from the HR2 region of HIV gp41, wherein the amino acid sequence comprises the HR2 leucine zipper-like motif, e.g., the HR2 leucine zipper-like motifs depicted in FIG. 1 or FIG. 2. In a preferred embodiment, the HIV fusion inhibitor peptide is between 14 and 60 amino acid residues in length. In one embodiment, the HIV fusion inhibitor peptide further comprises a N-terminal blocking group or C-terminal blocking group, or both; those terminal groups may include, but are not limited to: an amino group or an acetyl group at the N-terminus; and a carboxyl group or an amido group at the C-terminus.

Further antiviral peptides useful in the compositions and methods provided herein include HIV fusion inhibitor peptides having an amino acid sequence similar to SEQ ID NO:5, except that the HIV fusion inhibitor peptide amino acid sequence: (a) has more than one leucine zipper-like motif, and has at least one additional leucine other than a leucine needed to form a leucine zipper-like motif (i.e., other than at position 1 or 8 of a leucine zipper-like motif); or (b) has more than two leucine zipper-like motifs; and wherein the HIV fusion inhibitor peptide demonstrates an improvement in one or more biological properties. In one embodiment, the HIV fusion inhibitor peptide is between 14 and 60 amino acid residues in length. In one embodiment, the HIV fusion inhibitor peptide contains an amino acid sequence derived from the HR2 region of HIV gp41, wherein the amino acid sequence comprises the HR2 leucine zipper-like motif, e.g., the HR2 leucine zipper-like motifs depicted in FIG. 1 or FIG. 2.

Further antiviral peptides useful in the compositions and methods provided herein include peptides exemplified by SEQ ID NOs:9, 10, 14, and 15, or an HIV fusion inhibitor peptide containing between one to three amino acid differences as compared to any one of SEQ ID NOs:9, 10, 14, and 15.

Further antiviral peptides useful in the compositions and methods provided herein include HIV fusion inhibitor peptides which are similar in amino acid sequence to a base amino acid sequence of SEQ ID NO:5 except that, as compared to the base amino acid sequence, the HIV fusion inhibitor peptide amino acid sequence has more than two leucine zipper-like motif in its amino acid sequence; wherein the HIV fusion inhibitor peptide demonstrates an unexpected, improvement in one or more biological properties. Further antiviral peptides useful in the compositions and methods provided herein include peptides exemplified by SEQ ID NOs:11-13, or HIV fusion inhibitor peptides containing between one to three amino acid differences as compared to any one of SEQ ID NOs:11-13.

The HIV fusion inhibitor peptides described herein can routinely be produced via well-known methods, including the recombinant expression of nucleic acids encoding the peptide. For example, cells engineered to recombinantly express an HIV fusion inhibitor peptide can be cultured for an appropriate time and under appropriate conditions such that the peptide is expressed, and the peptide can be obtained therefrom. The HIV fusion inhibitor peptides described herein can also be produced via synthesis methods. In one embodiment, the peptides are assembled via linear synthesis methods. In other embodiments, the peptides are assembled using a fragment condensation approach from 2 or more peptide fragments. In one embodiment, a 2 fragment condensation approach is used wherein fragments AA(1-26) and AA(27-37) are covalently coupled and assembled with AA(38) to yield the HIV fusion inhibitor peptide having an amino acid sequence of SEQ ID NO:9. In a further embodiment, a 3 fragment approach is used wherein fragments AA(1-12), AA(13-26) and AA(27-37) are covalently coupled and assembled with AA(38) to yield the HIV fusion inhibitor peptide having an amino acid sequence of SEQ ID NO:9.

Specific peptide fragments that can be used in the compositions described herein follow. Each peptide fragment can serve as an intermediate that can be covalently coupled with one or more other peptide fragments in a group of peptide fragments to yield the HIV fusion inhibitor peptide having an amino acid sequence of either SEQ ID NO:9, SEQ ID NO:10, SEQ ID NO:11, SEQ ID NO:12, SEQ ID NO:13, SEQ ID NO:14, or SEQ ID NO:15. In one embodiment, the peptide fragments, within a group of peptide fragments, are coupled in a solution phase process in a manner to result in the desired HIV fusion inhibitor peptide having an amino acid sequence of SEQ ID NO:9, SEQ ID NO:10, SEQ ID NO:11, SEQ ID NO:12, SEQ ID NO:13, SEQ ID NO:14, or SEQ ID NO:15. Also useful in the compositions provided herein are HIV fusion inhibitor peptides produced by synthesizing its constituent peptide fragments, and then assembling the peptide fragments to form the HIV fusion inhibitor peptide, wherein the HIV fusion inhibitor peptide has an amino acid sequence of either SEQ ID NO:9, SEQ ID NO:10, SEQ ID NO:11, SEQ ID NO:12, SEQ ID NO:13, SEQ ID NO:14, or SEQ ID NO:15.

In one embodiment, provided herein are methods for preparing a peptide comprising spray drying the peptide. For example, a peptide can be dissolved (e.g., in water) at a pH less than 4 or greater than 6, wherein an appropriate acid or base, such as 1N NaOH or 1N HCl, is used to adjust pH, followed by spraying the peptide solution through an atomizing nozzle into a heated chamber. Dried peptide particles can be collected manually.

In another embodiment, the spray drying method described above further comprises adding an excipient to the spray drying solution, thereby incorporating the excipient and the peptide. Examples of illustrative excipients include, but are not limited to, fillers, extenders, diluents, wetting agents, solvents, emulsifiers, preservatives, flavors, absorption enhancers, sustained-release matrices, coloring agents, and macromolecular substances such as albumin, or substances such as amino acids and sugars.

In another embodiment, provided herein are methods for preparing a peptide comprising spraying a peptide solution through an atomizing nozzle (in a manner similar to spray drying) into another solution (e.g., a metal salt solution, such as a zinc salt, iron salt or calcium salt solution). The resulting suspension can then be centrifuged, the supernatant decanted, and the precipitate frozen. The precipitate can be lyophilized and passed through a 200 μm screen.

In another embodiment, provided herein are methods for preparing a peptide comprising salt or pH precipitation, wherein a peptide is dissolved (e.g., in water) at a pH less than 4 or greater than 6, wherein an appropriate acid or base, such as 1N NaOH or 1N HCl, is used to adjust pH to between about 4 and 6 or between about 4.8 and 5.2. In a particular embodiment, this method comprises adding a salt solution or strong acid/base solution to the peptide solution to cause precipitation. In another embodiment, this method further comprises collection of the precipitate by centrifugation, drying of the precipitate by lyophilization and optional passage of the precipitate through a 200 μm screen to control particle size.

Without being limited by theory, it is thought that the process and reagents used to prepare a peptide used in a composition provided herein can result in desirable or improved pharmacokinetic parameters (e.g., amount or duration of release of a bioactive molecule) useful for certain patients or diseases. In particular, the manner (e.g., sprayed and/or precipitated) in which a metal (e.g., zinc, iron or calcium) is incorporated into a peptide precipitate can affect the pharmacokinetic parameters of the resulting composition.

In one embodiment, increasing the metal content (e.g., zinc content) during the peptide precipitation process can decrease Cₘₐₓ, increase tₘₐₓ and increase t_{0.01}. In another embodiment, adding a metal salt (e.g., zinc sulfate) as a lyophilized salt to a low-metal (e.g., low-zinc) precipitate can decrease Cₘₐₓ, increase tₘₐₓ and increase t_{0.01}.

In another embodiment, the solution from which the peptide is precipitated can affect Cₘₐₓ and tₘₐₓ. For example, precipitating peptide from 50:50 methanol:water can decrease Cₘₐₓ and increase tₘₐₓ relative to precipitating peptide from water alone.

In another embodiment, the manner in which the peptide is prepared can affect Cₘₐₓ and tₘₐₓ. For example, sprayed precipitates can increase Cₘₐₓ and increase tₘₐₓ relative to non-sprayed precipitates.

### Methods of Use

Further provided herein are methods of using the compositions provided herein. In one embodiment, the compositions are used as a part of a therapeutic regimen, for example, an antiviral therapeutic regimen. In certain embodiments, such a therapeutic regimen can, for example, be used for the therapy of HIV infection.

In one embodiment, provided herein is a method of using the compositions provided herein for inhibition of transmission of HIV to a target cell, comprising administering an amount of a composition provided herein to a patient such that the target cell is contacted with an amount of an active agent, e.g., an antiviral peptide, effective to inhibit infection of the cell by the virus. This method can, for example, be used to treat HIV-infected patients. In one embodiment, inhibiting transmission of HIV to a target cell comprises inhibiting gp41-mediated fusion of HIV-1 to a target cell and/or inhibiting syncytia formation between an HIV-infected cell and a target cell.

Also provided herein are methods of treating HIV infection (in one embodiment, HIV-1 infection) comprising administering to an HIV-infected patient a composition provided herein in an amount effective to treat the HIV infection. In one embodiment, the composition comprises an amount of an HIV fusion inhibitor peptide effective to inhibit transmission of HIV to a target cell, and/or an amount of an HIV fusion inhibitor peptide effective to inhibit gp41-mediated fusion of HIV to a target cell. These methods can, for example, be used to treat HIV-infected patients.

In a particular embodiment, provided herein are methods for ameliorating a symptom associated with an HIV infection, comprising administering to an HIV infected patient a composition comprising a solvent, a gelling material and a peptide selected from T20, T1249, T897, T2635, T999 and T1144, or a combination thereof.

Further provided herein are methods for the use of a composition containing an HIV fusion inhibitor peptide in the manufacture of a medicament for use in therapy of HIV infection (e.g., used in a method of inhibiting transmission of HIV, a method of inhibiting HIV fusion, and/or a method of treating HIV infection). The medicament can be in the form of a pharmaceutical composition comprising a bioactive molecule, such as an HIV fusion inhibitor peptide, a solvent, a gelling material and optionally one or more pharmaceutically acceptable carriers.

In one embodiment, the compositions provided herein can be administered by injection, such as subcutaneous injection.

In another embodiment, the compositions provided herein can be administered (e.g., by subcutaneous injection) once every 3, 5, 7, 10, 14, 17, 21, 28 or 60 days.

In another embodiment, the compositions provided herein can be administered (e.g., by subcutaneous injection) once, twice, three times or more per day for one or more days, one or more weeks, one or more months, or one or more years.

In another embodiment, the composition provided herein can be administered (e.g., by subcutaneous injection) one, two, three, four, five, six, seven or more times per week. In a specific embodiment, the compositions provided herein can be administered (e.g., by subcutaneous injection) once or twice per week. In another specific embodiment, the compositions provided herein are administered (e.g., by subcutaneous injection) twice every two weeks. Each administration can comprise one, two, three or more injections.

In one embodiment, the compositions provided herein are administered at a volume of 100 μl, 200 μl, 300 μl, 400 μl, 500 μl, 600 μl, 700 μl, 800 μl, 900 μl, 1000 μl or more.

In one embodiment, the compositions provided herein are delivered by injection by syringe, for example, syringes of 100 μl, 200 μl, 300 μl, 400 μl, 500 μl, 600 μl, 700 μl, 800 μl, 900 μl, 1000 μl or more in volume. In one embodiment, an auto-injector or pen device for delivery may be used. In another embodiment, the delivery device may be prefilled. In one particular embodiment, an AutoJect 2 by Owen Mumford may be used as a delivery syringe with 300 μl, 500 μl or 1000 μl volumes with a 30 gauge, 0.5 inch fixed needle may be used. In another particular embodiment, Becton Dickinson UltraFine syringes with 300 μl, 500 μl or 1000 μl volumes with a 30 gauge, 0.5 inch fixed needle may be used. In one embodiment, injection depth of drug delivery may be controlled.

In one particular embodiment, the compositions provided herein are administered once daily at a dosage of 50 mg/ml of active pharmaceutical ingredient, for example, SEQ ID NO:9, with a pharmaceutically acceptable carrier, for example, mannitol, in water for injection (at pH 7.4). In another particular embodiment, the compositions provided herein are administered once daily at a dosage of 125 mg/ml of active pharmaceutical ingredient, for example, SEQ ID NO:9, in water at pH 7.4.

### EXAMPLES

### Example 1

In the following examples, various biophysical parameters and biological parameters were assessed. The general methodologies for determining these parameters are as follows.

Peptides, including HIV fusion inhibitor peptides and base sequences, were synthesized on a peptide synthesizer using standard solid phase synthesis techniques and using standard FMOC peptide chemistry, or a combination of solid phase synthesis and solution phase synthesis as described in more detail in Example 3 herein. In this example, the HIV fusion inhibitor peptides could further comprise reactive functionalities; i.e., most were blocked at the N-terminus by an acetyl group and/or at the C-terminus by an amide group. After cleavage from the resin, the peptides were precipitated, and the precipitate was lyophilized. The peptides were then purified using reverse-phase high performance liquid chromatography; and peptide identity was confirmed with electrospray mass spectrometry.

Assessment of biophysical parameters included measurement of helicity and thermal stability. Helicity was assessed by circular dichroism (“CD”) as follows. Briefly, CD spectra were obtained using a spectrometer equipped with a thermoelectric temperature controller. The spectra was obtained at 25° C. with 0.5 nanometer (nm) steps from 200 to 260 nm, with a 1.5 nm bandwith, and a typical averaging time of 4 seconds/step. After the cell/buffer blank was subtracted, spectra were smoothed using a third-order least-squares polynomial fit with a conservative window size to give random residuals. Raw ellipticity values were converted to mean residue ellipticity using standard methods, and plotted was the wavelength (from 200 to 260 nm) versus [θ]×10−3 (degrees cm²/dmol). Percent helicity values were then calculated using standard methods (usually expressed as percent helicity at 10 μM, 25° C.). Assessment of thermal stability was performed by monitoring the change in CD signal at 222 nm as temperature was raised in 2° C. steps, with 1 minute equilibration times. The stability for each sample (e.g., HIV fusion inhibitor peptide), as represented by the Tm value, is the temperature corresponding to the maximum value of the first derivative of the thermal transition.

Assessment of biological properties included measurement of antiviral activity against HIV-1 strains. In determining antiviral activity (e.g., one measure being the ability to inhibit transmission of HIV to a target cell) of the HIV fusion inhibitor peptides, an in vitro assay which has been shown, by data generated using peptides derived from the HR regions of HIV gp41, to be predictive of antiviral activity observed in vivo was used. More particularly, antiviral activity observed using an in vitro infectivity assay (“Magi-CCR5 infectivity assay”; see, e.g., U.S. Pat. No. 6,258,782) has been shown to reasonably correlate to antiviral activity observed in vivo for the same HIV gp41 derived peptides (see, e.g., Kilby et al., 1998, Nature Med. 4:1302-1307). These assays score for reduction of infectious virus titer employing the indicator cell lines MAGI or the CCR5 expressing derivative cMAGI. Both cell lines exploit the ability of HIV-1 tat to transactivate the expression of a β-galactosidase reporter gene driven by the HIV-LTR. The β-gal reporter has been modified to localize in the nucleus and can be detected with the X-gal substrate as intense nuclear staining within a few days of infection. The number of stained nuclei can thus be interpreted as equal to the number of infectious virions in the challenge inoculum if there is only one round of infection prior to staining. Infected cells are enumerated using a CCD-imager and both primary and laboratory adapted isolates show a linear relationship between virus input and the number of infected cells visualized by the imager. In the MAGI and cMAGI assays, a 50% reduction in infectious titer (Vn/Vo=0.5) is significant, and provides the primary cutoff value for assessing antiviral activity (“IC50” is defined as the concentration of active ingredient resulting in a 50% reduction in infectious virus titer). Peptides tested for antiviral activity were diluted into various concentrations, and tested in duplicate or triplicate against an HIV inoculum adjusted to yield approximately 1500-2000 infected cells/well of a 48 well microtiter plate. The peptide (in the respective dilution) was added to the cMAGI or MAGI cells, followed by the virus inocula; and 24 hours later, an inhibitor of infection and cell-cell fusion (e.g., SEQ ID NO:2 (enfuvirtide)) was added to prevent secondary rounds of HIV infection and cell-cell virus spread. The cells were cultured for 2 more days, and then fixed and stained with the X-gal substrate to detect HIV-infected cells. The number of infected cells for each control and peptide dilution was determined with the CCD-imager, and then the IC50 was calculated (expressed in μg/ml).

Viruses resistant to the antiviral activity of a peptide consisting of a base sequence can be produced using standard laboratory methods. Basically, after calculating the IC50 and IC90, cells were mixed with virus and the peptide (e.g., at a concentration close to the IC90) in culture (including when the cells are split thereafter). The cultures are maintained and monitored until syncytia are present. Virus harvested from this first round of culture is used to infect cells in a second round of culture, with the peptide present in a higher concentration (2 to 4 times) than that used in the first round of culture. The second round of culture is maintained and monitored for presence of virus resistant to the antiviral activity of the peptide. Subsequent rounds of culture may be needed to finally generate a viral isolate resistant to the antiviral activity of the peptide (at a pre-determined level of the IC50 of the peptide against such isolate).

For determining pharmacokinetic properties, an HIV fusion inhibitor peptide or a base sequence from which an HIV fusion inhibitor peptide is derived was dosed intravenously in cynomolgus monkeys (Macaca fasicularis) (other animal models may be used for determining pharmacokinetic properties, as known in the art). At various times post-dose, blood samples were drawn and plasma isolated by centrifugation. Plasma samples were stored frozen until analysis by LC-MS (liquid chromatography/mass spectrometry) in the electrospray, positive-ion mode. An HIV fusion inhibitor or base sequence was eluted from a C18 or C8 HPLC column with a gradient of acetonitrile in a buffer of 10 mM ammonium acetate, pH 6.8. At the time of analysis, plasma samples were deproteinated with either two or three volumes of acetonitrile containing 0.5% formic acid. Duplicate calibration standards in cynomolgus plasma samples were prepared at the same time as the samples and analyzed before and after the samples containing either HIV fusion inhibitor peptide or base sequence. Pharmacokinetic properties were calculated from the plasma concentration-time data using either mono-exponential or bi-exponential mathematical models. Models were derived by non-linear least squares optimization. A 1/C² weighting of concentrations was used. The following equations were used to calculate area-under the plasma concentration vs. time curve (AUC), total body clearance (Cl), and terminal elimination half-life (t ½).

AUC=A/−a+B/−b

Where A and B are intercepts and a and b are the rate constants of the exponential equations describing the distribution and elimination phases, respectively. When mono-exponential models were used, the “A” and “a” properties were eliminated.

Cl=Dose/AUC (expressed in L/K/hr)

t ½=0.6903/b (expressed in hr)

### Example 2

For purposes of illustrating the embodiments provided herein, the base sequence has the following amino acid sequence (SEQ ID NO:5).

TTWEAWDRAIAEYAARIEALIRAAQEQQEKNEAALREL

In one embodiment, an HIV fusion inhibitor peptide comprises, as compared to a base sequence from which it is derived, more than 2 leucine zipper-like motifs. Examples of such HIV fusion inhibitor peptides include, but are not limited to, SEQ ID NO:11, SEQ ID NO:12, and SEQ ID NO:13; or an amino acid sequence having between one and three amino acid differences as compared to (e.g., at least a 92% identity with) any one of SEQ ID NO:11, SEQ ID NO:12, SEQ ID or NO:13; each HIV fusion inhibitor peptide has an amino acid sequence of between 3 and 5 leucine zipper-like motifs. The following illustration (I) shows amino acid sequences of HIV fusion inhibitor peptides with amino acid differences (as compared to the base sequence) using the one letter amino acid code (“L” for leucine, and “I” for isoleucine) under the amino acid position of the base sequence (as aligned using an “I”), and with the isoleucine and leucines involved in a leucine zipper-like motif (either at position 1 or 8 of the same leucine zipper-like motif, or position 8 of one leucine zipper-like motif and position 1 of an adjacent leucine zipper-like motif) underlined. Position 1 or 8 of one leucine zipper may also function as the opposite terminal position of another leucine zipper-like motif in a sequence, i.e., as position 8 of one motif and position 1 of another subsequent motif.

| | | |
|---|---|---|
| (I) | TTWEAWDRAIAEYAARIEALIRAAQEQQEKNEAALREL | SEQ ID NO: 5 |
| | \| \| | |
| | L L | SEQ ID NO: 11 |
| | I L | SEQ ID NO: 12 |
| | L I | SEQ ID NO: 13 |

In another embodiment, an HIV fusion inhibitor peptide comprises, as compared to a base sequence from which it is derived, more than 1 leucine zipper-like motif, as well as an additional leucine not involved in formation of a leucine zipper-like motif. Examples of such HIV fusion inhibitor peptides include, but are not limited to, SEQ ID NO:9, SEQ ID NO:10, SEQ ID NO:14, and SEQ ID NO:15; or an amino acid sequence having between one and three amino acid differences as compared to (e.g., at least a 92% identity with) any one of SEQ ID NO:9, SEQ ID NO:10, SEQ ID NO:14, or SEQ ID NO:15; and each HIV fusion inhibitor peptide amino acid sequence differing from the base sequence of SEQ ID NO:5 by containing more than 1 leucine zipper-like motif, and an additional leucine not involved in formation of a leucine zipper-like motif (i.e., other than at position 1 or 8 of a leucine zipper-like motif). In one embodiment, the non-leucine zipper-like motif leucine substitution replaces a isoleucine at the amino acid 21 position in the base sequence of SEQ ID NO:5, a substitution that provides a factor in promoting beneficial biological properties for these peptides. The following illustration (II) shows amino acid sequences of HIV fusion inhibitor peptides with amino acid differences (as compared to the base sequence) using the one letter amino acid code (“L” for leucine, and “I” for isoleucine) under the amino acid position of the base sequence (as aligned using an “I”), and with the isoleucine and leucines involved in a leucine zipper-like motif (either at position 1 or 8 of the same leucine zipper-like motif, or position 8 of one leucine zipper-like motif and position 1 of an adjacent leucine zipper-like motif) underlined. Italicized is a leucine not involved in formation of a leucine zipper-like motif.

| | | |
|---|---|---|
| (II) | TTWEAWDRAIAEYAARIEALIRAAQEQQEKNEAALREL | SEQ ID NO: 5 |
| | \| \| \| | |
| | L L | SEQ ID NO: 9 |
| | L L | SEQ ID NO: 10 |
| | L I | SEQ ID NO: 14 |
| | L I | SEQ ID NO: 15 |

The following illustration (III) presents a summary comparison of the “base sequence” SEQ ID NO:5 to peptides SEQ ID NOs:9-15 of the present disclosure which demonstrate improved biological properties relative to SEQ ID NO:5. Amino acid substitutions in each of SEQ ID NOs:9-15 relative to the SEQ ID NO:5 base sequence are underlined and in bold.

(III)

SEQ ID NO: 5

T T W E A W D R A I A E Y A A R I E A L I R A A Q
E Q Q E K N E A A L R E L

SEQ ID NO: 9

T T W E A W D R A I A E Y A A R I E A L L R A L Q
E Q Q E K N E A A L R E L

SEQ ID NO: 10

T T W E A W D R A I A E Y A A R I E A L L R A A Q
E Q Q E K L E A A L R E L

SEQ ID NO: 11

T T W E A W D R A I A E Y A A R I E A L I R A L Q
E Q Q E K L E A A L R E L

SEQ ID NO: 12

T T W E A W D R A I A E Y A A R I E A L I R A I Q
E Q Q E K L E A A L R E L

SEQ ID NO: 13

T T W E A W D R A I A E Y A A R I E A L I R A L Q
E Q Q E K I E A A L R E L

SEQ ID NO: 14

T T W E A W D R A I A E Y A A R I E A L L R A I Q
E Q Q E K N E A A L R E L

SEQ ID NO: 15

T T W E A W D R A I A E Y A A R I E A L L R A A Q
E Q Q E K I E A A L R E L

With reference to Table 1, an HIV fusion inhibitor peptide according to this present invention was compared to synthetic peptides which have the same base sequence, but differ in amino acid sequence (as compared to SEQ ID NOs:9-15) and that have anti-HIV activity. The comparison includes biophysical parameters and biological activity parameters, as determined using the methodology described in Example 1 herein. In determining biological activity, as assessed by antiviral activity, a viral isolate is utilized which is resistant to the antiviral activity of some peptides known to inhibit HIV-mediated fusion (the resistant viral isolate being designated as “Res” in Table 1).

TABLE 1
Biophysical and Biological (antiviral activity) Parameters

| | | | Antiviral | Antiviral |
|---|---|---|---|---|
| SEQ ID | Helicity | | Activity (μg/ml) | Activity (μg/ml) |
| NO: | (%) | Tm (° C.) | HIV-IIIB IC50 | HIV-Res IC50 |
| 5 | 71 | 42 | <0.10 | <0.10 |
| 6 | 97 | 65 | <0.10 | >0.10 |
| 7 | 84 | 75 | <0.10 | >0.10 |
| 8 | 99 | 46 | <0.10 | Not tested |
| 9 | 61 | 62 | <0.10 | <0.10 |
| 10 | 77 | 75 | <0.10 | <0.10 |

SEQ ID NO:6 and SEQ ID NO:7 differ from base sequence SEQ ID NO:5 by a single leucine substitution (at position 24 or position 31, respectively); as seen above in Table 1, this substitution does not markedly impact antiviral activity, yet this substitution leads to an improvement in half-life (see Table 2, below). SEQ ID NO:6 is similar to an HIV fusion inhibitor peptide according to the present invention having SEQ ID NO:9, except that the amino acid sequence of SEQ ID NO:9 has one further amino acid difference, a leucine in amino acid position 21 (whereas SEQ ID NO:6 has an isoleucine in amino acid position 21). With reference to Table 1, the leucine for isoleucine substitution in SEQ ID NO:9 delivers a reduction (from 97% to 61%) in helicity, while maintaining a good resistance profile (activity against the resistant viral isolate “Res”) as compared to a peptide of SEQ ID NO:6. Similarly, SEQ ID NO:7 is an amino acid sequence similar to an HIV fusion inhibitor peptide according to the present invention having SEQ ID NO:10, except that the amino acid sequence of SEQ ID NO:10 has one amino acid difference, a leucine in amino acid position 21 (whereas SEQ ID NO:7 has an isoleucine in amino acid position 21). With reference to Table 1, the leucine for isoleucine substitution in SEQ ID NO:10 results in a reduction (from 84% to 77%) in helicity, while maintaining a good resistance profile (activity against the resistant viral isolate “Res”) as compared to a peptide of SEQ ID NO:7. Thus, Table 1 demonstrates improved properties for SEQ ID NO:9 and 10 relative to SEQ ID NOs:6-7.

Illustrated in this embodiment are pharmacokinetic properties of an HIV fusion inhibitor peptide according to the present invention as compared to a base amino acid sequence. Using methods for assessing pharmacokinetic properties as previously described in more detail in Example 1, Table 2 illustrates pharmacokinetic properties of a representation of HIV fusion inhibitor peptides according to the present invention as compared to the pharmacokinetic properties of a base sequence SEQ ID NO: 5.

TABLE 2

| SEQ ID NO: | Clearance (L/kg/hr) | Half-life (t½; hr) |
|---|---|---|
| 5 | >0.04 | 6 |
| 6 | <0.02 | 15 |
| 7 | <0.02 | 17 |
| 8 | >0.04 | 7 |
| 9 | <0.02 | 12 |
| 10 | <0.02 | 21 |

As shown in Table 2, each of SEQ ID NOs:6, 7, 9, and 10 exhibit an increased biological half-life (“t½”). SEQ ID NO:8, which contains a leucine at amino acid position 21, but not at either of amino acid positions 24 or 31, does not exhibit the dramatic increase in half-life exhibited by the peptides of SEQ ID NOs:6, 7, 9, and 10.

For formulating an HIV fusion inhibitor into a pharmaceutically acceptable carrier in producing a pharmaceutical formulation, stability in aqueous solution may be an important parameter, particularly if the pharmaceutical formulation is to be administered parenterally. It is noted that an HIV fusion inhibitor peptide according to the present invention demonstrates improvement in stability in aqueous solutions at physiological pH. For example, synthetic peptides having an amino acid sequence of SEQ ID NO:2, SEQ ID NO:5, and an HIV fusion inhibitor peptide having an amino acid sequence of SEQ ID NO:9, were each individually tested for solubility by adding the peptide at a concentration of 10 mg/ml to phosphate-buffered saline (PBS), and by measuring (e.g., by HPLC) at different time points over a period of 1 week (168 hours) the amount of peptide remaining in solution at a range of about pH 7.3 to about pH 7.5 at 37° C. A solution containing SEQ ID NO:2 becomes unstable after just several hours (minimal peptide detected in solution). In contrast, 90% or more of the HIV fusion inhibitor peptide having an amino acid sequence of SEQ ID NO:9 remains detectable in solution at a time point of 1 week, whereas less than 80% of a peptide having the amino acid sequence of SEQ ID NO:5 remains detectable in solution at a time point of 1 week.

### Example 3

Biological properties of the HIV fusion inhibitor peptides provided herein have been compared with other recognized, effective antiviral agents, including SEQ ID NO: 2 (enfuvirtide). In particular, in vitro resistance comparison studies were performed between the novel SEQ ID NO:9 compound of interest and established antiviral agent SEQ ID NO:2, described in detail as follows: MT2 cells were infected with virus isolates (IIIB, 030, 060 and 098) and cultured in increasing concentrations of SEQ ID NO:2 (enfuvirtide) or SEQ ID NO:9 to select for resistance. The initial peptide concentration was approximately 2 times the IC₅₀ of each peptide against the corresponding wild type isolate. Peptide concentrations were maintained by adding fresh peptide every 1-3 days. Cultures were monitored for cytopathic effect (CPE) using standard techniques and when maximal CPE was achieved, a small aliquot of virus was used for subsequent rounds of infection. Peptide concentrations were increased 2 to 4-fold depending on the length of time in culture when compared to the growth rate of wild type virus. During the course of selection, peptide-free virus stocks were also collected. Peptide-free virus stocks were characterized for gp41 genotypic changes by dideoxy sequencing chemistries and phenotypic susceptibility was determined using a cMAGI infectivity assay.

The results of comparison between in vitro selections using SEQ ID NO:2 and SEQ ID NO:9 are shown in Table 3. These data show that SEQ ID NO:9 selections were in culture an average of 3 times longer than SEQ ID NO:2 selections, resulting in lower fold changes in IC50 (42-fold for SEQ ID NO:2 compared to 24-fold for SEQ ID NO:9). SEQ ID NO:9 selections required more mutations (geometric mean of 3.6) to achieve these lower fold changes than did SEQ ID NO. 2 (geometric mean of 1.7). The longer days in culture, lower fold changes and higher number of mutations required to effect the lower fold changes, all indicate that SEQ ID NO:9 exhibits a higher barrier to development of resistance in vitro compared to SEQ ID NO:2. That is, these results indicate that HIV resistance to SEQ ID NO:9 takes longer to arise than resistance to SEQ ID NO:2. Based on previous studies of HIV resistance development conducted on other peptides, e.g., SEQ ID NO:2 and T1249, one would expect that the in vitro results presented herein should reasonably correlate with results in vivo.

TABLE 3
Comparison between SEQ ID NO: 2 (enfuvirtide) and SEQ ID NO: 9 in vitro
selections

| | | | | | Fold | # of |
|---|---|---|---|---|---|---|
| Starting Virus | Peptide | Days in | Starting IC50 | Ending IC50 | Change in | Mutations |
| Isolate | (SEQ ID NO) | Culture | (ng/mL) | (ng/mL) | IC50 | Acquired |
| IIIB | 2 | 62 | 6 | 163 | 27 | 2 |
| 584.000030 | 2 | 46 | 42 | 798 | 19 | 2 |
| 584.000060 | 2 | 46 | 10 | 68 | 7 | 2 |
| 584.000098 | 2 | 45 | 50 | 45575 | 912 | 1 |
| Geometric Mean | 2 | 49 | 19 | 797 | 42 | 1.7 |
| IIIB | 9 | 168 | 12 | 2768 | 231 | 4 |
| 584.000030 | 9 | 77 | 28 | 113 | 4 | 2 |
| 584.000060 | 9 | 173 | 8 | 208 | 26 | 4 |
| 584.000098 | 9 | 173 | 37 | 521 | 14 | 5 |
| Geometric Mean | 9 | 140 | 18 | 429 | 24 | 3.6 |

Based on previous studies of HIV resistance development conducted on other peptides, e.g., SEQ ID NO:2 and T1249, one would expect that the in vitro results presented herein should reasonably correlate with results in vivo (see, e.g., Melby et al., 2006, AIDS Research and Human Retroviruses 22(5):375-385; Greenberg & Cammack, 2004, J. Antimicrobial Chemotherapy 54:333-340; Sista et al., 2004, AIDS 18:1787-1794).

### Example 4

In general, an HIV fusion inhibitor peptide provided herein can be synthesized by each of two methods. A first method is by linear synthesis using standard solid-phase synthesis techniques and using standard Fmoc peptide chemistry or other standard peptide chemistry (using chemical protecting groups, or CPGs). A second method for synthesis of an HIV fusion inhibitor peptide provided herein is by a fragment condensation approach. Briefly, 2 or more fragments, each fragment containing a respective portion of the complete amino acid sequence of the HIV fusion inhibitor peptide to be produced, is synthesized. In the synthesis of a fragment, if desired, incorporated may be an amino acid having its free amine (e.g., side chain amine) chemically protected by a chemical protecting agent. The fragments are then assembled (covalently coupled together in a manner and order) such that the HIV fusion inhibitor peptide is produced (with the proper amino acid sequence).

With respect to peptide synthesis, the individual peptide fragments themselves, and the HIV fusion inhibitor peptide provided herein which is produced from a combination of a group of peptide fragments, can each be made using techniques known to those skilled in the art for synthesizing peptide sequences. For example, in one approach, the peptide fragments can be synthesized in solid phase, and then combined in solution phase, in a process of assembly to produce the resultant HIV fusion inhibitor peptide. In another approach, solution phase synthesis can be used to produce the peptide fragments, which then are combined in solid phase in a process of assembly to produce the HIV fusion inhibitor peptide. In still another approach, each peptide fragment can be synthesized using solid phase synthesis, and then combined in solid phase in a process of assembly to produce the complete amino acid sequence of the HIV fusion inhibitor peptide. In one embodiment, each peptide fragment is produced using solid phase synthesis known to those skilled in the art. In another embodiment, an HIV fusion inhibitor peptide having the amino acid sequence of SEQ ID NO:9 is produced using an assembly process that combines solid phase and solution phase techniques using a group of peptide fragments. For example, a group of peptide fragments comprises between 2 to 4 peptide fragments that are synthesized, and then assembled, to complete the synthesis of an HIV fusion inhibitor peptide provided herein. Based on the teachings herein, it is apparent to one skilled in the art that this approach of fragment assembly can be used, and has been used, for some of the HIV fusion inhibitor peptides having an amino acid sequence of any one of SEQ ID NOs:9-16.

To illustrate production of an HIV fusion inhibitor peptide provided herein by the fragment condensation approach, peptides fragments, in a group of peptide fragments, were covalently coupled in assembling the peptide fragments in a method of synthesizing an HIV fusion inhibitor peptide having an amino acid sequence of SEQ ID NO:9. The peptide fragments provided herein can include, but are not limited to, those having the amino acid sequences depicted in the following Table 4. Certain peptide fragment(s) provided herein can be used to the exclusion of other peptide fragment(s). The corresponding amino acids in SEQ ID NO:9 of each peptide fragment are also indicated; thus, it is shown that each peptide fragment is made up of a number of contiguous amino acids of the amino acid sequence of SEQ ID NO:9.

TABLE 4

| SEQ | | Amino acid |
|---|---|---|
| ID | | positions in |
| NO: | Amino acid sequence | SEQ ID NO: 9 |
| 17 | TTWEAWDRAIAE | 1-12 |
| 18 | YAARIEALLRALQE | 13-26 |
| 19 | QQEKNEAALRE | 27-37 |
| 20 | QQEKNEAALREL | 27-38 |
| 21 | TTWEAWDRAIA | 1-11 |
| 22 | EYAARIEALLRALQE | 12-26 |
| 23 | TTWEAWDRAI | 1-10 |
| 24 | AEYAARIEALLRALQE | 11-26 |
| 25 | TTWEAWDRA | 1-9 |
| 26 | IAEYAARIEALLRALQE | 10-26 |
| 27 | TTWEAWDR | 1-8 |
| 28 | AIAEYAARIEALLRALQE | 9-26 |
| 29 | TTWEAWDRAIAEYAARIEAL | 1-20 |
| 30 | LRALQEQQEKNEAALRE | 21-37 |
| 31 | LRALQEQQEKNEAALREL | 21-38 |
| 32 | TTWEAWDRAIAEYAARIE | 1-18 |
| 33 | ALLRALQEQQEKNEAALRE | 19-37 |
| 34 | ALLRALQEQQEKNEAALREL | 19-38 |
| 35 | YAARIE ALLRALQEQQEKNEAALREL | 13-38 |
| 36 | EYAARIE ALLRALQEQQEKNEAALREL | 12-38 |
| 37 | AEYAARIE ALLRALQEQQEKNEAALREL | 11-38 |
| 38 | IAEYAARIE ALLRALQEQQEKNEAALREL | 10-38 |
| 39 | AIAEYAARIE ALLRALQEQQEKNEAALREL | 9-38 |
| 40 | TTWEAWDRAIAEYAARIEALLRALQE | 1-26 |
| 57 | TTWEAWDRAIAEYAARIEALLRALQEQQEKNEAA | 1-37 |
| | LRE | |
| 58 | AARIEALLRALQEQQEKNEAALRE | 14-37 |
| 59 | RIEALLRALQEQQEKNEAALRE | 16-37 |
| 60 | QEQQEKNEAALREL | 25-38 |
| 61 | LQEQQEKNEAALREL | 24-37 |
| 62 | EQQEKNEAALREL | 26-38 |
| 63 | TTWEAWDRAIAEYAARIEALLRALQ | 1-25 |
| 64 | TTWEAWDRAIAEYAARIEALLRAL | 1-24 |
| 65 | TTWEAWDRAIAEYAARIEALLR | 1-22 |
| 66 | TTWEAWDRAIAEYAARIEALL | 1-21 |
| 67 | TTWEAWDRAIAEYAARIEA | 1-19 |
| 68 | TTWEAWDRAIAEYAARI | 1-17 |
| 69 | TTWEAWDRAIAEYAAR | 1-16 |
| 70 | TTWEAWDRAIAEYAA | 1-15 |
| 71 | TTWEAWDRAIAEYA | 1-14 |
| 72 | TYWEAWDRAIAEY | 1-13 |
| 73 | EL | 37-38 |
| 74 | AARIEALLRALQE | 14-26 |
| 75 | ARIEALLRALQE | 15-26 |
| 76 | RIEALLRALQE | 16-26 |
| 77 | EALLRALQE | 18-26 |

Further provided herein are particular groups of peptide fragments which act as intermediates in a method of synthesis of an HIV fusion inhibitor peptide having the amino acid sequence of SEQ ID NO:9. The groups of peptide fragments provided herein include Groups 1-16, as designated in Table 5 (the numbering of a group is for ease of description only). Certain group(s) of peptide fragments can be used to the exclusion of other group(s) of peptide fragments.

TABLE 5

| | | |
|---|---|---|
| | | Amino acid |
| Group | | positions in |
| Number | Peptide fragments | SEQ ID NO: 9 |

| | | | |
|---|---|---|---|
| 1 | TTWEAWDRAIAE | (SEQ ID NO: 17) | 1-12 |
| | YAARIEALLRALQE | (SEQ ID NO: 18) | 13-26 |
| | QQEKNEAALRE | (SEQ ID NO: 19) | 27-37 |
| 2 | TTWEAWDRAIAE | (SEQ ID NO: 17) | 1-12 |
| | YAARIEALLRALQE | (SEQ ID NO: 18) | 13-26 |
| | QQEKNEAALREL | (SEQ ID NO: 20) | 27-38 |
| 3 | TTWEAWDRAIAEYAARIEAL | (SEQ ID NO: 29) | 1-20 |
| | LRALQEQQEKNEAALRE | (SEQ ID NO: 30) | 21-37 |
| 4 | TTWEAWDRAIAEYAARIEAL | (SEQ ID NO: 29) | 1-20 |
| | LRALQEQQEKNEAALREL | (SEQ ID NO: 31) | 21-38 |
| 5 | TTWEAWDRAIA | (SEQ ID NO: 21) | 1-11 |
| | EYAARIEALLRALQE | (SEQ ID NO: 22) | 12-26 |
| | QQEKNEAALRE | (SEQ ID NO: 19) | 27-37 |
| 6 | TTWEAWDRAI | (SEQ ID NO: 23) | 1-10 |
| | AEYAARIEALLRALQE | (SEQ ID NO: 24) | 11-26 |
| | QQEKNEAALRE | (SEQ ID NO: 19) | 27-37 |
| 7 | TTWEAWDRA | (SEQ ID NO: 25) | 1-9 |
| | IAEYAARIEALLRALQE | (SEQ ID NO: 26) | 10-26 |
| | QQEKNEAALRE | (SEQ ID NO: 19) | 27-37 |
| 8 | TTWEAWDR | (SEQ ID NO: 27) | 1-8 |
| | AIAEYAARIEALLRALQE | (SEQ ID NO: 28) | 9-26 |
| | QQEKNEAALRE | (SEQ ID NO: 19) | 27-37 |
| 9 | TTWEAWDRAIA | (SEQ ID NO: 21) | 1-11 |
| | EYAARIEALLRALQE | (SEQ ID NO: 22) | 12-26 |
| | QQEKNEAALREL | (SEQ ID NO: 20) | 27-38 |
| 10 | TTWEAWDRAI | (SEQ ID NO: 23) | 1-10 |
| | AEYAARIEALLRALQE | (SEQ ID NO: 24) | 11-26 |
| | QQEKNEAALREL | (SEQ ID NO: 20) | 27-38 |
| 11 | TTWEAWDRA | (SEQ ID NO: 25) | 1-9 |
| | IAEYAARIEALLRALQE | (SEQ ID NO: 26) | 10-26 |
| | QQEKNEAALREL | (SEQ ID NO: 20) | 27-38 |
| 12 | TTWEAWDR | (SEQ ID NO: 27) | 1-8 |
| | AIAEYAARIEALLRALQE | (SEQ ID NO: 28) | 9-26 |
| | QQEKNEAALREL | (SEQ ID NO: 20) | 27-38 |
| 13 | TTWEAWDRAIAEYAARIE | (SEQ ID NO: 32) | 1-18 |
| | ALLRALQEQQEKNEAALRE | (SEQ ID NO: 33) | 19-37 |
| 14 | TTWEAWDRAIAEYAARIE | (SEQ ID NO: 32) | 1-18 |
| | ALLRALQEQQEKNEAALREL | (SEQ ID NO: 34) | 19-38 |
| 15 | TTWEAWDRAIAEYAARIEALLRALQE | (SEQ ID NO: 40) | 1-26 |
| | QQEKNEAALRE | (SEQ ID NO: 19) | 27-37 |
| 16 | TTWEAWDRAIAEYAARIEALLRALQE | (SEQ ID NO: 40) | 1-26 |
| | QQEKNEAALREL | (SEQ ID NO: 20) | 27-38 |

Thus, in one embodiment, provided herein are methods, peptide fragments, and groups of peptide fragments that can be used to synthesize an HIV fusion inhibitor peptide having the amino acid sequence of SEQ ID NO:9. It is also apparent from the description herein that such methods, peptide fragments, and groups of peptide fragments can be used to synthesize an HIV fusion inhibitor Peptide having the amino acid sequence of SEQ ID NO:9, wherein the HIV fusion inhibitor peptide contains one or more chemical groups:

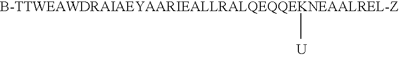

wherein one or more of the amino terminal end, carboxyl terminal end, or side chain free reactive functionality (e.g., an epsilon amine of an internal lysine) is modified by a chemical group (B, U, Z; wherein B, U, and Z may be the same chemical group or different chemical groups) which may include, but is not limited to, one or more of: a reactive functionality, a chemical protecting group (CPG), and a linker. Techniques useful for introducing a chemical group at the N-terminus of a peptide fragment, or the C-terminus of a peptide fragment, at a free amine at an internal amino acid, or a combination thereof, are well known in the art. Illustrative examples of protected peptide fragments (peptide fragments having one or more chemical groups), as related to the production of an HIV fusion inhibitor peptide having an amino acid sequence of SEQ ID NO:9, include, but are not limited to, the peptide fragments listed in Table 6.

TABLE 6

| | | Amino acid |
|---|---|---|
| SEQ | | positions in |
| ID NO: | Amino acid sequence | SEQ ID NO: 9 |
| 17 | Ac-TTWEAWDRAIAE | 1-12 |
| 18 | CPG-YAARIEALLRALQE | 13-26 |
| 19 | CPG-QQEKNEAALRE | 27-37 |
| 19 | 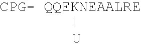 | 27-37 |
| 20 | QQEKNEAALREL—NH2 | 27-38 |
| 29 | Ac-TTWEAWDRAIAEYAARIEAL | 1-20 |
| 30 | CPG-LRALQEQQEKNEAALRE | 21-37 |
| 31 | LRALQEQQEKNEAALRE L—NH2 | 21-38 |
| 21 | Ac-TTWEAWDRAIA | 1-11 |
| 22 | CPG-EYAARIEALLRALQE | 12-26 |
| 23 | Ac-TTWEAWDRAI | 1-10 |
| 24 | CPG-AEYAARIEALLRALQE | 11-26 |
| 25 | Ac-TTWEAWDRA | 1-9 |
| 26 | CPG-IAEYAARIEALLRALQE | 10-26 |
| 27 | Ac-TTWEAWDR | 1-8 |
| 28 | CPG-AIAEYAARIEALLRALQE | 9-26 |
| 32 | Ac-TTWEAWDRAIAEYAARIE | 1-18 |
| 33 | CPG-ALLRALQEQQEKNEAALRE | 19-37 |
| 34 | ALLRALQEQQEKNEAALRE L—NH2 | 19-38 |

Ac-acetyl group, NH₂-amide group (but can be another chemical group as described in more detail in the “Definitions” section herein); CPG is chemical protecting group (e.g., Fmoc or other N-terminal chemical protecting group, as described in more detail in the “Definitions” section herein); U is as defined above.

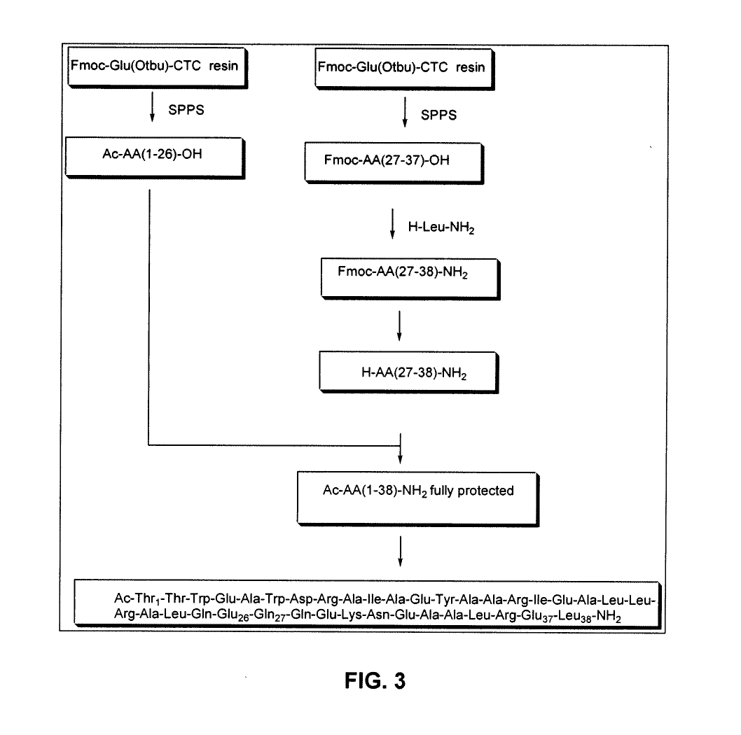

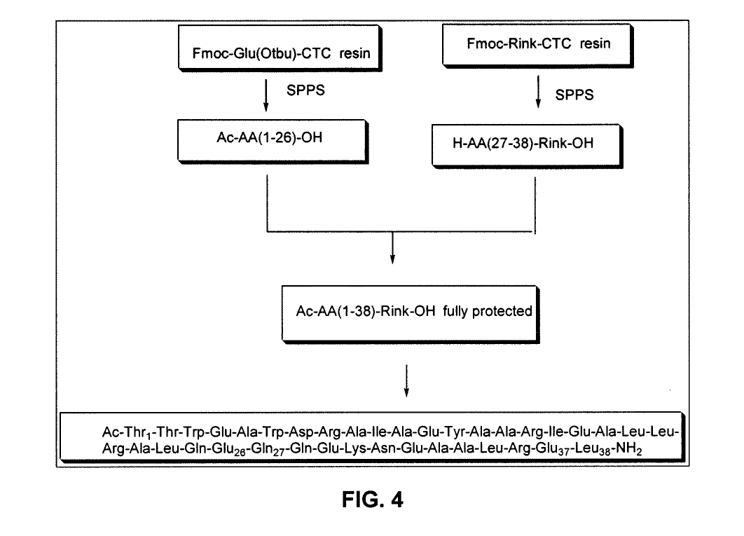

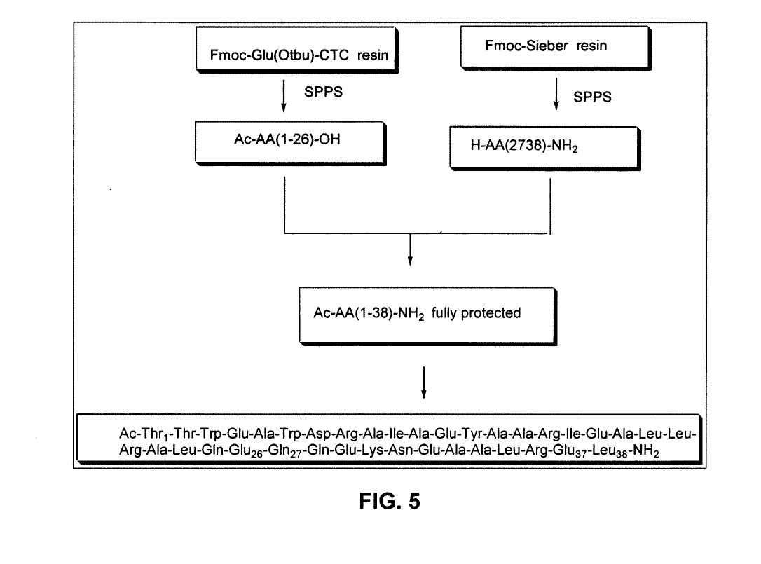

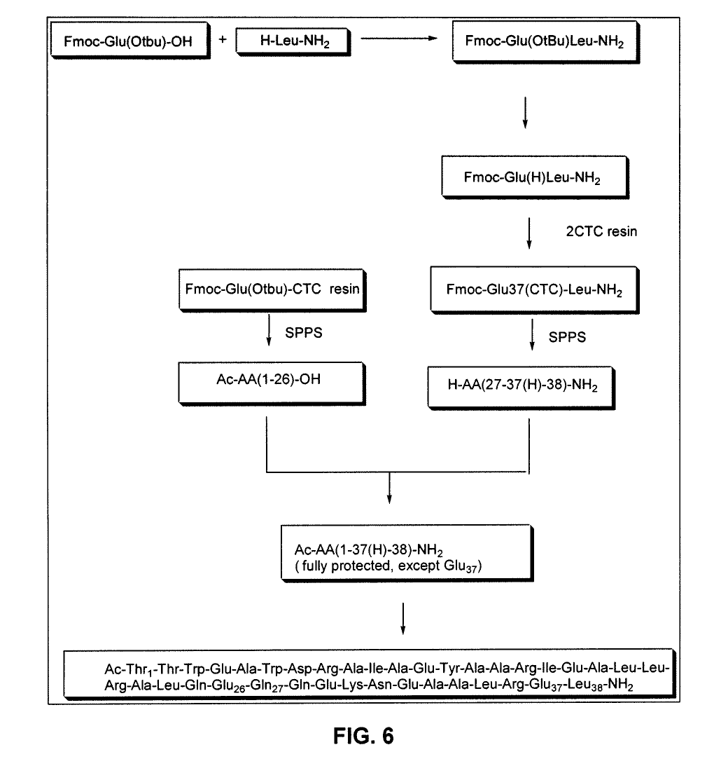

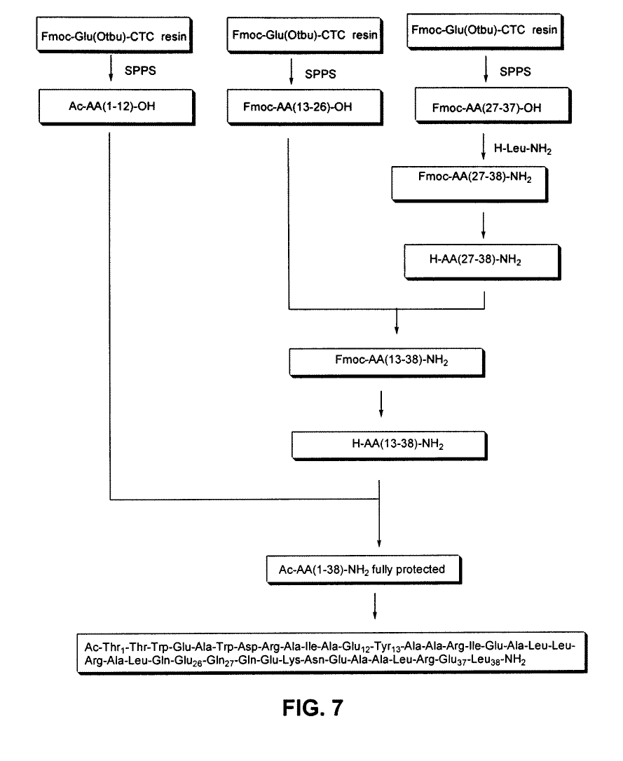

SEQ ID NO:9 was initially synthesized linearly to produce initial research quantities. A two fragment synthesis was first used to produce large amounts of SEQ ID NO:9, and due to the efficiency introduced in the process by a two fragment approach, this route (FIG. 3) was used for the first GMP synthesis of toxicology and clinical material. Three fragment approaches (see, e.g., FIG. 7) were also implemented. Leading routes of synthesis include, for example, a 2 fragment approach using fragments of amino acid positions 1-26 and amino acid positions 27-37, and a 3 fragment approach using fragments of amino acid positions 1-12, amino acid positions 13-26, and amino acid positions 27-37. Various modifications to the originally linear synthesis of SEQ ID NO:9 have been examined (Example 12). SEQ ID NO:9 was assembled linearly, for example, on Sieber amide resin, on rink-loaded CTC resin, and on Glu37 side chain loaded resin. Further examples of linear synthesis methods used to assemble SEQ ID NO:9 include, for example, synthesis using rink-loaded CTC (FIG. 4), Sieber resin (FIG. 5) and Glu-loaded CTC (FIG. 6).

### Example 5

In referring to Table 5 (Group 1 or Group 2) and FIG. 7, illustrated is a method for synthesis of an HIV fusion inhibitor peptide having the amino acid sequence of SEQ ID NO:9 using 3 specific peptide fragments (e.g., SEQ ID NOs:17-19+Leu; or SEQ ID NOs:17, 18, and 20), and using a fragment condensation approach involving combining the 3 peptide fragments to produce the HIV fusion inhibitor peptide. Each of these peptide fragments demonstrated physical properties and solubility characteristics that make them preferred peptide fragments (relative to certain two fragment approaches) to be used in a method for synthesis of an HIV fusion inhibitor peptide having the amino acid sequence of SEQ ID NO:9 in high yield and high purity, and further requires only one loaded resin as starting material (in simplifying the method for synthesis). A peptide fragment having the amino acid sequence of SEQ ID NO:17, and comprising the first 12 amino acids of SEQ ID NO:9 (see FIG. 7, “AA(1-12)”, was synthesized by standard solid phase synthesis (using a super acid sensitive resin; e.g., 4-hydroxymethyl-3-methoxyphenoxy-butyric acid resin, or 2-chlorotrityl chloride resin-“CTC”, FIG. 7), with acetylation of (“Ac”, as a chemical group) the N-terminus, while having a carboxyl group (—COOH) at the C-terminus (see, FIG. 7, “Ac-AA(1-12)-OH”). A peptide fragment having the amino acid sequence of SEQ ID NO:18, and comprising amino acids 13-26 of SEQ ID NO:9 (see, FIG. 7, “AA(13-26)”), was synthesized by standard solid phase synthesis with Fmoc at the N-terminus (as a chemical protecting group), and —OH at the C-terminus (see, FIG. 7, “Fmoc-AA(13-26)-OH”). A peptide fragment having the amino acid sequence of SEQ ID NO:19, and comprising amino acids 27-37 of SEQ ID NO:9 (see, FIG. 7, “AA(27-37)”), was synthesized by standard solid phase synthesis with Fmoc at the N-terminus (as a chemical protecting group), and —OH at the C-terminus (see, FIG. 7, “Fmoc-AA(27-37)-OH”). Each peptide fragment was cleaved from the resin used for its solid phase synthesis by cleavage reagents, solvents, and techniques well known to those skilled in the art. Each peptide fragment was then isolated by removing the majority of above mentioned solvents by distillation and precipitating the peptide fragment by the addition of water with or without an alcohol containing-cosolvent. The resulting solid was isolated by filtration, washed, reslurried in water or alcohol/water, refiltered, and dried in a vacuum oven.

As shown in FIG. 7, a peptide fragment was produced by solution phase synthesis, wherein the peptide fragment having the amino acid sequence of SEQ ID NO:19 (see, FIG. 7, “Fmoc-AA(27-37)-OH”) was chemically coupled to Leu, amino acid 38 of SEQ ID NO:9, which has been amidated in solution phase to result in a peptide fragment having the amino acid sequence of SEQ ID NO:20 (comprising amino acids 27-38 of SEQ ID NO:9) with amidation of the C-terminus (as a chemical group) (see, FIG. 7, “Fmoc-AA(27-38)-NH₂”). In one method of synthesis, amidated peptide fragments provided herein, including but not limited to peptide fragment H-AA(27-38)-NH₂, can be synthesized directly using an amide resin. In summary of this solution phase reaction, the carboxy terminus of isolated peptide fragment Fmoc-AA(27-37)-OH is converted to an active ester of HOBT (1-hydroxybenzotriazole hydrate), 6-Cl HOBt*H₂O, or HOAT (1-Hydroxy-7-azabenzotriazole) using HBTU (O-benzotriazol-1-yl-N,N,N′,N′-tetramethyluronium hexafluorophosphate) or TBTU (O-benzotriazol-1-yl-N,N,N′,N′-tetramethyltetrafluoro-borate) and HOBT, 6-Cl HOBT, or HOAT, respectively, in the presence of DIEA (diisopropylethyl amine) and leucine amide (e.g., a combination of coupling reagents and racemization suppressants). The reaction is run in a polar, aprotic solvent such as DMF (dimethyl formamide), DMAc (dimethylacetamide) or NMP (N-methylpyrrolidinone) at 0 to 30° C. At the completion of the coupling reaction, piperidine, potassium carbonate, DBU or other bases known to those in the art are added to the reaction with or without an additional cosolvent to effect removal of the terminal Fmoc protecting groups. At the completion of the reaction, alcohol or a water miscible solvent and/or water are added to precipitate the peptide fragment having the amino acid sequence of SEQ ID NO:20 with amidation of the C-terminus (H-AA(27-38)-NH₂).

As schematically illustrated in FIG. 7, to produce peptide fragment H-AA(27-38)-NH₂ using a peptide fragment Fmoc-AA(27-37)-OH combined with leucine amide (see, FIG. 3, “H-Leu-NH₂”) in a solution phase process, the peptide fragment Fmoc-AA(27-37)-OH (571 g, 205 mmol, 1 eq), H-Leu-NH₂ (32.0 g, 246 mmol, 1.2 eq), and 6-Cl HOBT (41.7 g, 246 mmol, 1.2 eq) were added to DMF (4568 ml, 8 vol), treated with DIEA (53.6 ml, 307.5 mmol, 1.5 eq) and stirred at room temperature until dissolved (about 20 minutes). The solution was cooled, and TBTU (79.0 g, 246 mmol, 1.2 eq) was added. The reaction was stirred for at 0° C., then at 25° C. When analysis by HPLC showed the reaction was complete, piperidine (81 ml, 820 mmol, 4 eq) was added to remove the Fmoc protecting group (other bases such as potassium carbonate, DBU, etc., could be used) of the peptide fragment Fmoc-AA(27-38)-NH₂. The reaction was stirred at 30° C. until shown to be complete by HPLC. Then the reaction mixture was cooled below 5° C. and pre-cooled water (8 vol, 4568 mL) was slowly added keeping the temperature of the resulting slurry below 10° C. The suspension was stirred for 30 minutes, and then filtered and washed twice with 25% ethanol/water (2284 mL, 4 vol each). Residual piperidine and piperidine dibenzylfulvene was removed by reslurries in ethanol/water (with or without dilute acid) and/or MTBE/heptane or other similar solvent mixtures. As shown in FIG. 7, the result was a preparation of isolated peptide fragment H-AA(27-38)-NH₂.

As illustrated in FIG. 7, a solution phase reaction was then performed in which peptide fragment H-AA(27-38)-NH₂ (SEQ ID NO:20) is combined with peptide fragment Fmoc-AA(13-26)-OH (SEQ ID NO:18) and deprotected to yield a peptide fragment H-AA(13-38)-NH₂ (SEQ ID NO:35 with chemical groups at each of the N-terminus and C-terminus).

Peptide fragment Fmoc-AA(13-26)-OH (460 g, 167 mmol, 1 eq), peptide fragment H-AA(27-38)-NH₂ (460 g, 172 mmol, 1.03 eq), and 6-Cl HOBT (34 g, 200 mmol, 1.2 eq) were added to DMF (6900 ml, 15 vol), treated with DIEA (47 mL, 267 mmol, 1.6 eq), and stirred to dissolve all solids. The resulting solution was cooled to below 5° C. To the reaction was added TBTU (64 g, 200 mmol, 1.2 eq), and the reaction was stirred at 0° C. and then at 25° C. Once analysis by HPLC showed the reaction was complete, piperidine (58 ml, 668 mmol, 4 eq) was added to remove the Fmoc, and the reaction stirred until shown complete by HPLC. The solution was cooled to below 5° C. and water (6900 mL, 15 vol) was slowly added at a rate such that the temperature did not rise above 10° C. After stirring the resulting suspension for 30 minutes, the solids were collected by filtration and washed with water (twice, 2300 mL, 5 vol each), and dried. Residual piperidine and piperidine dibenzylfulvene was removed by reslurries in ethanol/water (with or without dilute acid) and/or MTBE/heptane or other similar solvent mixtures. The solids were collected by filtration, washed, and dried affording H-AA(13-38)-NH₂ (SEQ ID NO:35) as a substantially pure white solid as determined by high performance liquid chromatography (HPLC) analysis for purity.

As illustrated in FIG. 7, peptide fragment H-AA(13-38)-NH₂ (SEQ ID NO:35) was then assembled in a solution phase reaction with peptide fragment Ac-(1-12)-OH (SEQ ID NO:17) to yield an HIV fusion inhibitor peptide having the amino acid sequence of SEQ ID NO:9 (see, e.g., FIG. 7, Ac-(1-38)-NH₂). Peptide fragment Ac-AA(1-12)-OH (130 g, 58.5 mmol, 1 eq) was milled to a fine powder and mixed with peptide fragment H-AA(13-38)-NH₂ (303 g, 58.5 mmol, 1 eq). This mixture was slowly added to a warm solution of 2:1 DCM/DMF (20 vol, 2600 mL) and DIEA (25.5 mL, 146 mmol, 2.5 eq). HOAT (15.9 g, 117 mmol, 2.0 eq) was added and the mixture was stirred to dissolve all solids. The resulting solution was cooled to below 5° C. and TBTU (28.2 g, 87.8 mmol, 1.5 eq) was added. The solution was stirred for 30 minutes at 0° C. and then at 25° C., until HPLC showed the reaction was complete. The solution was warmed to 30-35° C. and additional DCM (13 vol, 1740 mL) followed by H₂O (1820 mL, 14 vol) was added. The mixture was stirred for 5 min and then the layers were allowed to separate. The aqueous layer was removed and replaced with fresh H₂O (1820 mL, 14 vol). The separation was repeated a total of 5 times. The organic layer was distilled to approximately ⅓ its original volume and isopropyl alcohol (IPA; 1820 mL, 14 vol) was added. The distillation was continued to remove the remaining DCM. The resulting slurry was cooled to below 5° C. and H₂O (1820 mL, 14 vol) was slowly added. The solids formed were collected by filtration, washed twice with H₂O (520 mL, 4 vol each) and dried affording a preparation of isolated HIV fusion inhibitor peptide Ac-AA(1-38)-NH₂ (SEQ ID NO:9), as determined by HPLC analysis for purity.

As shown in FIG. 7, the side chain chemical protecting groups of HIV fusion inhibitor peptide Ac-AA(1-38)-NH₂ may be removed by acidolysis or any other method known to those skilled in the art for deprotecting a peptide by removing side chain chemical protecting groups. In this example, HIV fusion inhibitor peptide Ac-AA(1-38)-NH₂ (60 g, 8.1 mmol) was treated with TFA (trifluoracetic acid):DTT(dithiothreitol):water (90:10:5; 570 ml) and stirred at room temperature for 6 hours. The solution was cooled to below 10° C., and pre-cooled MTBE (25 vol, 1500 ml) was slowly added at a rate such that the temperature remained below 10° C. The resulting solids were collected by filtration, washed with MTBE, and dried. The resulting powder was then slurried in acetonitrile (ACN; 10 vol, 600 mL) and the pH was adjusted to between 4 and 5 with DIEA and acetic acid to decarboxylate the peptide. Once this was complete by HPLC, the solids were collected by filtration, washed with ACN, and dried to yield a preparation of deprotected and decarboxylated peptide, which was then purified by HPLC or other suitable chromatographic techniques to yield a preparation of isolated protected HIV fusion inhibitor peptide having an amino acid sequence of SEQ ID NO:9.

### Example 6

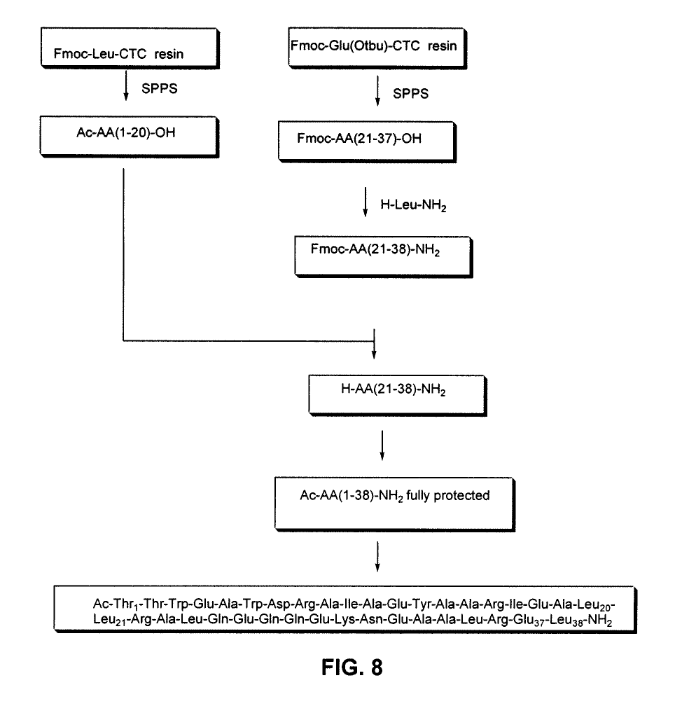

In referring to Table 5 (Group 3 and Group 4) and FIG. 8, illustrated is a method for synthesis of a HIV fusion inhibitor peptide having the amino acid sequence of SEQ ID NO:9 using 2 specific peptide fragments (e.g., SEQ ID NOs:29 & 30+Leu; or SEQ ID NOs:29 & 31), and using a fragment assembly approach involving combining 2 peptide fragments by chemically coupling (“assembling”) them to produce HIV fusion inhibitor peptide having an amino acid sequence of SEQ ID NO:9. Each of these peptide fragments demonstrated physical properties and solubility characteristics that make them useful and/or preferable in a method for synthesis, using 2 peptide fragments, of an HIV fusion inhibitor peptide having the amino acid sequence of SEQ ID NO:9 in high yield and purity. In selecting peptide fragments to be used in a two fragment assembly approach, it was discovered that having leucine and/or glutamic acid residues at the point of juncture between the two fragments being assembled together (e.g., the C-terminal amino acid of a peptide fragment having the amino acid sequence of SEQ ID NO:29, and the N-terminal amino acid of a peptide fragment having the amino acid sequence of SEQ ID NO:31) favored assembly in the high yield and the degree of purity obtained.

A peptide fragment having the amino acid sequence of SEQ ID NO:29, and comprising the first 20 amino acids of SEQ ID NO:9 (“AA(1-20)”), was synthesized by standard solid phase synthesis, with acetylation of (“Ac”, as a chemical group) the N-terminus, while having a hydroxyl group (—OH) at the C-terminus (see, Table 6; also referred to herein as “Ac-AA(1-20)-OH”). A peptide fragment having the amino acid sequence of SEQ ID NO:30, and comprising amino acids 21-37 of SEQ ID NO:9 (“AA(21-37)”), was synthesized by standard solid phase synthesis with Fmoc at the N-terminus (as a chemical protecting group), and —OH at the C-terminus (see, Table 6; also referred to herein as “Fmoc-AA(21-37)-OH”).

As shown in Table 5, Groups 3 and 4, and FIG. 8, a peptide fragment was produced by solution phase synthesis, when the peptide fragment Fmoc-AA(21-37)-OH was chemically coupled to Leu, amino acid 38 of SEQ ID NO:9 which had been amidated, in solution phase to result in a peptide fragment having the amino acid sequence of SEQ ID NO:31 (comprising amino acids 21-38 of SEQ ID NO:9) with amidation of the C-terminus (as a chemical group) (“Fmoc-AA(21-38)-NH₂”). To produce peptide fragment Fmoc-AA(21-38)-NH₂ using a peptide fragment Fmoc-AA(21-37)-OH combined with leucine (“H-Leu NH₂”) in a solution phase process, the peptide fragment Fmoc-AA(21-37)-OH (30 g, 7.43 mmol, 1.0 eq), H-Leu-NH₂*HCl (1.36 g, 8.16 mmol, 1.2 eq), and HOAT (1.52 g, 11.2 mmol, 1.5 eq) were dissolved in DMF (450 ml, 15 vol), treated with DIEA (6.5 ml, 37.3 mmol, 5 eq), and stirred at room temperature until dissolved (about 30 minutes). The solution was cooled to 0±5° C., and TBTU (2.86 g, 8.91 mmol, 1.2 eq) was added, stirred for 5 minutes at 0±5° C., and then allowed to react at 25±5° C. for 2 hours or until the reaction was shown complete by HPLC.

The Fmoc chemical protecting group of the peptide fragment Fmoc-AA(21-38)-NH₂ was then removed prior to isolation of the fragment H-AA(21-38)-NH₂. Piperidine (7.3 mL, 73.8 mmol, 10 eq) was added and the solution was stirred for 1 hour at 25±5° C. or until analysis by HPLC showed that substantially all the Fmoc was removed from the peptide fragment. The reactor was cooled, and water (1000 ml, 30 vol) was added, and the free-flowing slurry was stirred 30 minutes at less than 10° C., and then isolated by filtration. The collected solid was washed with 1:1 EtOH/water and dried in a vacuum oven at 35±5° C. The peptide fragment is then reslurried in 1:1 EtOH/water (450 mL, 15 vol) for 3 hours. The solids were collected and dried. Then the peptide fragment was slurried in 3:1 hexanes:MTBE (450 mL, 15 vol) overnight, and then isolated by filtration and redried. The MTBE reslurry may be repeated if necessary to remove additional piperidine. The result is a preparation of isolated peptide fragment H-AA(21-38)-NH₂ (see FIG. 8).

A solution phase reaction was then performed in which peptide fragment H-AA(21-38)-NH₂ (SEQ ID NO:31) was combined with peptide fragment Ac-AA(1-20)-OH (SEQ ID NO:29) to yield an HIV fusion inhibitor peptide having the amino acid sequence of SEQ ID NO:9 (see, FIG. 8, Ac-(1-38)-NH₂). Peptide fragment H-AA(21-38)-NH₂ (3.40 g, 0.86 mmol, 1 eq), peptide fragment Ac-AA(1-20)-OH (3.00 g, 0.86 mmol, 1.0 eq), and HOAT (0.177 g, 1.3 mmol, 1.5 eq) and DIEA (0.599 ml, 3.44 mmol, 4 eq) were dissolved in DMAc (dimethyl acetamide; 100 ml, 33 vol), cooled to 0±5° C. Added to the reaction was TBTU (0.331 g, 1.03 mmol, 1.2 eq). The reaction was stirred for 5 minutes at 0±5° C. and at 25±5° C. for 3 hours or until the reaction was shown to be complete by HPLC. The reactor was cooled, and water (200 ml, 66 vol) was slowly added. A slurry was formed and stirred at less than 10° C. for at least 30 minutes. The solid was isolated by filtration and washed with additional water. The collected solid was dried in a vacuum oven at 35±5° C. The result was a preparation of fully protected, isolated HIV fusion inhibitor peptide Ac-AA(1-38)-NH₂ (SEQ ID NO:9), as determined by HPLC analysis for purity. The HIV fusion inhibitor peptide was then deprotected (by removing the side chain chemical protecting groups) and decarboxylated (at the tryptophan residues) by using the methods described herein in Example 5, or any other method known to those skilled in the art, for deprotection and decarboxylation, and then purified (e.g., by HPLC). The result was a preparation (deprotected and decarboxylated) of HIV fusion inhibitor peptide having an amino acid sequence of SEQ ID NO:9 (in this illustration, acetylated at the N-terminus, and amidated at the C-terminus).

Using similar techniques and conditions, additional fragment assembly approaches, involving 2 fragment assembly or 3 fragment assembly, have been used to produce an HIV fusion inhibitor peptide having an amino acid sequence of SEQ ID NO:9 (see, for example, Tables 4 and 5). It is understood from the descriptions herein that preferred peptide fragments, used to produce an HIV fusion inhibitor peptide having an amino acid sequence of SEQ ID NO:9 by the method of the present invention, may be used to the exclusion of peptide fragments other than the preferred peptide fragments. Likewise, a preferred group of peptide fragments, used to produce an HIV fusion inhibitor peptide having an amino acid sequence of SEQ ID NO:9 by the method of the present invention, may be used to the exclusion of groups of peptide fragments other than the preferred group of peptide fragments.

### Example 7

Another embodiment of the present invention relates to methods, peptide fragments, and groups of peptide fragments that may be used to synthesize an HIV fusion inhibitor peptide having the amino acid sequence of SEQ ID NO:10. It is also apparent from the description herein that such methods, peptide fragments, and groups of peptide fragments may be used to synthesize an HIV fusion inhibitor peptide having the amino acid sequence of SEQ ID NO:10, wherein the HIV fusion inhibitor peptide contains one or more chemical groups:

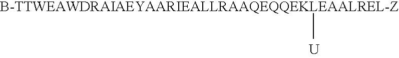

wherein either or both of the amino terminal end or carboxyl terminal end is modified by a chemical group (B, U, Z; wherein B, U, and Z may be the same chemical group or different chemical groups) which may include, but is not limited to, one or more of: a reactive functionality, a chemical protecting group (CPG), and a linker. Illustrative examples of peptide fragments, groups of peptide fragments, and protected peptide fragments (peptide fragments having one or more chemical groups), as related to the production of an HIV fusion inhibitor peptide having an amino acid sequence of SEQ ID NO:10, include, but are not limited to, those listed in Tables 7, 8, & 9, respectively.

TABLE 7

| SEQ | | Amino acid |
|---|---|---|
| ID | | positions in |
| NO: | Amino acid sequence | SEQ ID NO: 10 |
| 17 | TTWEAWDRAIAE | 1-12 |
| 41 | YAARIEALLRAAQE | 13-26 |
| 42 | QQEKLEAALRE | 27-37 |
| 43 | QQEKLEAALREL | 27-38 |
| 21 | TTWEAWDRAIA | 1-11 |
| 44 | EYAARIEALLRAAQE | 12-26 |
| 23 | TTWEAWDRAI | 1-10 |
| 45 | AEYAARIEALLRAAQE | 11-26 |
| 25 | TTWEAWDRA | 1-9 |
| 46 | IAEYAARIEALLRAAQE | 10-26 |
| 27 | TTWEAWDR | 1-8 |
| 47 | AIAEYAARIEALLRAAQE | 9-26 |
| 29 | TTWEAWDRAIAEYAARIEAL | 1-20 |
| 48 | LRAAQEQQEKLEAALRE | 21-37 |
| 49 | LRAAQEQQEKLEAALREL | 21-38 |
| 32 | TTWEAWDRAIAEYAARIE | 1-18 |
| 50 | ALLRAAQEQQEKLEAALRE | 19-37 |
| 51 | ALLRAAQEQQEKLEAALREL | 19-38 |
| 52 | YAARIEALLRAAQEQQEKLEAALREL | 13-38 |
| 53 | EYAARIEALLRAAQEQQEKLEAALREL | 12-38 |
| 54 | AEYAARIE ALLRAAQEQQEKLEAALREL | 11-38 |
| 55 | IAEYAARIE ALLRAAQEQQEKLEAALREL | 10-38 |
| 56 | AIAEYAARIEALLRAAQEQQEKLEAALREL | 9-38 |
| 78 | WQEWEQKITALLEQAQIQQEKNEYELQKLDKWAS | |
| | LWEWF | |
| 79 | YTSLIHSLIEESQNQQEKNEQELLELDKWASLWN | |
| | WFNIT | |
| 80 | YQEWERKVDFLEENITALLEEAQIQQEKNMYELQ | |
| | KL | |

Further provided herein are particular groups of peptide fragments which act as intermediates in a method of synthesis of an HIV fusion inhibitor peptide having the amino acid sequence of SEQ ID NO:10. The groups of peptide fragments provided herein include Groups 1-14, as designated in Table 8 (the numbering of a group is for ease of description only). Certain group(s) of peptide fragments can be used to the exclusion of other group(s) of peptide fragments.

TABLE 8

| | | |
|---|---|---|
| | | Amino acid |
| Group | | positions in |
| Number | Peptide fragments | SEQ ID NO: 10 |

| | | | |
|---|---|---|---|
| 1 | TTWEAWDRAIAE | (SEQ ID NO: 17) | 1-12 |
| | YAARIEALLRAAQE | (SEQ ID NO: 41) | 13-26 |
| | QQEKLEAALRE | (SEQ ID NO: 42) | 27-37 |
| 2 | TTWEAWDRAIAE | (SEQ ID NO: 17) | 1-12 |
| | YAARIEALLRAAQE | (SEQ ID NO: 41) | 13-26 |
| | QQEKLEAALREL | (SEQ ID NO: 43) | 27-38 |
| 3 | TTWEAWDRAIAEYAARIEAL | (SEQ ID NO: 29) | 1-20 |
| | LRAAQEQQEKLEAALRE | (SEQ ID NO: 48) | 21-37 |
| 4 | TTWEAWDRAIAEYAARIEAL | (SEQ ID NO: 29) | 1-20 |
| | LRAAQEQQEKLEAALREL | (SEQ ID NO: 49) | 21-38 |
| 5 | TTWEAWDRAIA | (SEQ ID NO: 21) | 1-11 |
| | EYAARIEALLRAAQE | (SEQ ID NO: 44) | 12-26 |
| | QQEKLEAALRE | (SEQ ID NO: 42) | 27-37 |
| 6 | TTWEAWDRAI | (SEQ ID NO: 23) | 1-10 |
| | AEYAARIEALLRAAQE | (SEQ ID NO: 45) | 11-26 |
| | QQEKLEAALRE | (SEQ ID NO: 42) | 27-37 |
| 7 | TTWEAWDRA | (SEQ ID NO: 25) | 1-9 |
| | IAEYAARIEALLRAAQE | (SEQ ID NO: 46) | 10-26 |
| | QQEKLEAALRE | (SEQ ID NO: 42) | 27-37 |
| 8 | TTWEAWDR | (SEQ ID NO: 27) | 1-8 |
| | AIAEYAARIEALLRAAQE | (SEQ ID NO: 47) | 19-26 |
| | QQEKLEAALRE | (SEQ ID NO: 43) | 27-37 |
| 9 | TTWEAWDRAIA | (SEQ ID NO: 21) | 1-11 |
| | EYAARIEALLRAAQE | (SEQ ID NO: 44) | 12-26 |
| | QQEKLEAALREL | (SEQ ID NO: 43) | 27-38 |
| 10 | TTWEAWDRAI | (SEQ ID NO: 23) | 1-10 |
| | AEYAARIEALLRAAQE | (SEQ ID NO: 45) | 11-26 |
| | QQEKLEAALREL | (SEQ ID NO: 43) | 27-38 |
| 11 | TTWEAWDRA | (SEQ ID NO: 25) | 1-9 |
| | IAEYAARIEALLRAAQE | (SEQ ID NO: 46) | 10-26 |
| | QQEKLEAALREL | (SEQ ID NO: 43) | 27-38 |
| 12 | TTWEAWDR | (SEQ ID NO: 27) | 1-8 |
| | AIAEYAARIEALLRAAQE | (SEQ ID NO: 47) | 9-26 |
| | QQEKLEAALREL | (SEQ ID NO: 43) | 27-38 |
| 13 | TTWEAWDRAIAEYAARIE | (SEQ ID NO: 32) | 1-18 |
| | ALLRAAQEQQEKLEAALRE | (SEQ ID NO: 50) | 19-37 |
| 14 | TTWEAWDRAIAEYAARIE | (SEQ ID NO: 32) | 1-18 |
| | ALLRAAQEQQEKLEAALREL | (SEQ ID NO: 51) | 19-38 |

TABLE 9

| | | Amino acid |
|---|---|---|
| SEQ | | positions in |
| ID NO: | Amino acid sequence | SEQ ID NO: 10 |
| 17 | Ac-TTWEAWDRAIAE | 1-12 |
| 41 | CPG-YAARIEALLRAAQE | 13-26 |
| 42 | CPG-QQEKLEAALRE | 27-37 |
| 42 | 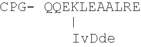 | 27-37 |
| 43 | QQEKLEAALREL—NH₂ | 27-38 |
| 29 | Ac-TTWEAWDRAIAEYAARIEAL | 1-20 |
| 48 | CPG-LRAAQEQQEKLEAALRE | 21-37 |
| 49 | LRAAQEQQEKLEAALRE L—NH₂ | 21-38 |
| 21 | Ac-TTWEAWDRAIA | 1-11 |
| 44 | CPG-EYAARIEALLRAAQE | 12-26 |
| 23 | Ac-TTWEAWDRAI | 1-10 |
| 45 | CPG-AEYAARIEALLRAAQE | 11-26 |
| 25 | Ac-TTWEAWDRA | 1-9 |
| 46 | CPG-IAEYAARIEALLRAAQE | 10-26 |
| 27 | Ac-TTWEAWDR | 1-8 |
| 47 | CPG-AIAEYAARIEALLRAAQE | 9-26 |
| 32 | Ac-TTWEAWDRAIAEYAARIE | 1-18 |
| 50 | CPG-ALLRAAQEQQEKLEAALRE | 19-37 |
| 51 | ALLRAAQEQQEKLEAALRE L—NH₂ | 19-38 |

In referring to Table 8 (Group 3 and Group 4), illustrated is a method for synthesis of an HIV fusion inhibitor peptide having the amino acid sequence of SEC) ID NO:10 using 2 specific peptide fragments (e.g., SEQ ID NOs:29 & 48+Leu; or SEQ ID NOs:29 & 49), and using a fragment assembly approach involving combining 2 peptide fragments by chemically coupling (“assembling”) them to produce an HIV fusion inhibitor peptide having an amino acid sequence of SEQ ID NO:10. To produce peptide fragment having an amino acid sequence of SEQ ID NO:49 (“Fmoc-AA(21-38)-NH₂”), using a peptide fragment having an amino acid sequence of SEQ ID NO:48 (“Fmoc-AA(21-37)-OH”) combined with leucine (“H-Leu-NH₂”) in a solution phase process, the peptide fragment Fmoc-AA(21-37)-OH (30.01 g, 7.98 mmol, 1.0 eq), H-Leu-NH₂*HCl (1.48 g, 8.78 mmol, 1.1 eq), and HOAT (1.63 g, 11.97 mmol, 1.5 eq) were dissolved in DMF (450 ml, 15 vol), treated with DIEA (7.0 ml, 39.91, mmol, 5 eq) and stirred at room temperature until dissolved (about 30 minutes), The solution was cooled to 0±5° C., and TBTU (3.09 g, 9.58 mmol, 1.2 eq) was added, stirred for 5 minutes at 0±5° C., and then allowed to react at 25±5° C. for 2 hours or until the reaction was shown complete by HPLC.

The Fmoc chemical protecting group of the peptide fragment Fmoc-AA(21-38)-NH₂ was then removed prior to isolation of the fragment H-AA(21-38)-NH₂. Piperidine (8.0 mL, 79.8 mmol, 10 eq) was added and the solution was stirred for 1.5 hours at 25±5° C. or until analysis by HPLC showed that substantially all the Fmoc was removed from the peptide fragment. The reactor was cooled, and water (1000 ml, 30 vol) was added, and the free-flowing slurry was stirred 30 minutes at less than 10° C., and then isolated by filtration. The collected solid was washed with 1:3 EtOH/water and dried in a vacuum oven at 35±5° C. The peptide fragment is then reslurried in 1:3 EtOH/water (400 mL, 13 vol) for 3 hours. The solids were collected and dried, and then the peptide fragment was slurried in 3:1 hexanes:MTBE (400 mL, 13 vol) overnight, isolated by filtration and redried. The MTBE reslurry may be repeated if necessary to remove additional piperidine. The result is a preparation of isolated peptide fragment H-AA(21-38)-NH₂ (see Table 9, SEQ ID NO:49).

A solution phase reaction was then performed in which peptide fragment H-AA(21-38)-NH₂ (SEQ ID NO:49) is combined with peptide fragment Ac-AA(1-20)-OH (SEQ ID NO:29, Table 9) to yield an HIV fusion inhibitor peptide having the amino acid sequence of SEQ ID NO:10 (see, e.g., Ac-(1-38)-NH₂). Peptide fragment H-AA(21-38)-NH₂ (3.14 g, 0.86 mmol, 1 eq), peptide fragment Ac-AA(1-20)-OH (3.00 g, 0.86 mmol, 1.0 eq), and HOAT (0.18 g, 1.3 mmol, 1.5 eq) and DIEA (0.599 ml, 3.44 mmol, 4 eq) were dissolved in DMAc (100 ml, 33 vol), cooled to 0±5° C. Added to the reaction was TBTU (0.331 g, 1.03 mmol, 1.2 eq). The reaction was stirred for 5 minutes at 0±5° C. and at 25±5° C. for 3 hours or until the reaction was shown to be complete using HPLC. The reactor was cooled, and water (250 ml, 83 vol) was slowly added. A slurry was formed and stirred at less than 10° C. for at least 30 minutes. The solid was isolated by filtration and washed with additional water. The collected solid dried in a vacuum oven at 35±5° C. The result was a preparation of fully protected, isolated HIV fusion inhibitor peptide Ac-AA(1-38)-NH₂ (SEQ ID NO:10), as determined by HPLC analysis for purity. The HIV fusion inhibitor peptide Ac-AA(1-38)-NH₂ was then deprotected (by removing the side chain chemical protecting groups) and decarboxylated (at the tryptophan residues) by using the methods described herein in Example 4, or any other method known to those skilled in the art, for deprotection and decarboxylation. Following purification, the result was a preparation (deprotected and decarboxylated) of isolated HIV fusion inhibitor peptide having an amino acid sequence of SEQ ID NO:10 (acetylated at the N-terminus and amidated at the C-terminus), as determined using HPLC.

Using similar techniques and conditions, additional fragment assembly approaches, involving 2 fragment assembly or 3 fragment assembly, may be used to produce the HIV fusion inhibitor having an amino acid sequence of SEQ ID NO:10 (see, for example, Tables 8 and 9). It is understood from the descriptions herein that peptide fragments, used to produce an HIV fusion inhibitor peptide having an amino acid sequence of SEQ ID NO:10 by the methods provided herein, can be used to the exclusion of other peptide fragments. Likewise, a group of peptide fragments, used to produce an HIV fusion inhibitor peptide having an amino acid sequence of SEQ ID NO:10 by the methods provided herein, can be used to the exclusion of other groups of peptide fragments.

### Example 8

A two fragment approach using Ac-AA(1-26)-OH and Fmoc-AA(27-38)-OH in the synthesis of the SEQ ID NO:9 peptide has been developed. Fragments Ac-AA(1-26)-OH and Fmoc-AA(27-37)-OH of SEQ ID NO:9 were first synthesized by standard solid phase synthesis techniques.

For synthesis of Fmoc-AA (27-37)-OH, the resin (2-CTC, 2 kg, 3.1 mol active Cl) was swelled in DCM (10 vol, 20 L) for 15-30 min at 25±5° C. and then drained while a solution of Fmoc-Glu(Otbu)-OH*H₂O (0.45 eq, 619 g, 1.40 mol) and DIEA (3 eq to Glu, 4.19 mmol, 729 mL) was prepared in DCM (5 vol, 10 L). The resin was reslurried in DCM (5 vol, 10 L), the amino acid solution was added to the resin, and the slurry was agitated for 2 hours at 25±5° C. The vessel was drained and the resin was slurried in NMP (4 vol, 8 L). The end-capping solution 9:1 (v/v) MeOH/DIEA (6 vol, 12 L) was added and the slurry stirred for 40-50 min at 25±5° C. The vessel was drained and the resin was washed with DCM (2×6 vol, 900 mL each). The resin was then washed with NMP (4 vol, 600 mL) and stored at 10° C.

The resin was swelled in NMP (10 vol, 20 L) for 30-60 min at 25±5° C. and then drained. The assembly of the fragment was accomplished using deprotections with 2×20-40 min×5 vol of 10% PIP in NMP at 30±5° C. The resin was then washed with NMP until a negative Chloranil test was obtained. Couplings were initiated after preactivating in DMF (6 vol) using 1.5 eq protected amino acid, 1.5 eq 6-Cl HOBt, 1.7 eq DIEA, and 1.5 eq TBTU at 0-5° C. for 15 min. After adding the coupling solution a DMF wash (2 vol) was added and the reaction was allowed to proceed for up to 4 h at 30±5° C. until complete by the Kaiser test. If incomplete after 3 hours, a recouple was performed. Once the coupling was complete, the resin was washed with NMP (3×6 vol, 12 L) for 5 min each.

The completed resin-bound fragment was washed 2× with 8 volumes NMP (16 L) and 4× with 8 vol DCM (16 L). The resin was slurried in 2 vol (4 L) DCM and 1% TFA in DCM (5 vol, 10 L) precooled to <5° C. was added. The slurry was stirred for 5 min and then filtered into a carboy containing pyridine (1.26 vol relative to TFA, 126 mL). Another portion of 1% TFA in DCM (5 vol, 10 L) precooled to <5° C. was added, stirred, and drained as before (without the 2 vol preswell). An additional 3 TFA/DCM treatments were performed (5 total). The resin was slurried in DCM (5 vol, 10 L) for 5 min and drained and the filtrate was monitored for product by HPLC. DCM washes were continued until the amount of product in the wash was minimal. The DCM was removed from the pooled DCM filtrates by distillation until the remaining solution was less than 4 volume level (8 L). Isopropanol (8 vol. 16 L) was added and the distillation was continued until the residual DCM content in the IPA slurry was <1%. The concentrate was cooled to <10° C. and precooled water (8 vol, 16 L) was added with rapid agitation to precipitate the product, at a rate that kept the temperature below 10° C. The slurry was warmed to 10-15° C. and aged. The product was isolated by filtration and washed with water (2×4 vol). The solids were air dried and then reslurried in 20% IPA/water (10 vol). The product was reisolated by filtration, washed with water (2 vol), and dried in a vacuum oven at 30-40° C. to constant weight. 2618 g (72.6%) of a white powder was recovered.

For synthesis of the fragment Ac-AA(1-26)-OH, the resin (2-CTC, 1.5 kg, 2.3 mol active Cl) was loaded as above for fragment Fmoc-AA(27-37)-OH. The fragment Ac-AA(1-26)-Resin was assembled as described above and cleaved in a similar manner. 2841 g (61.6%) of a white powder was recovered.

The corresponding synthesis of the fragment H-AA(27-38)-NH₂ was produced by solution phase synthesis, when the peptide fragment Fmoc-AA(27-37)-OH was chemically coupled to H-Leu-NH₂ (amino acid 38) in solution phase to yield Fmoc-AA(27-38)-NH₂ followed by removal of the N-terminal Fmoc protecting group to yield H-AA(27-38)-NH₂. Alternate bases (K₂CO₃, DBU, N-methyl piperidine, and DEA (Diethylamine) can be used.

To produce peptide fragment Fmoc-AA(27-38)-NH₂ the peptide fragment Fmoc-AA(27-37)-OH (2618 g, 940 mmol, 1.0 eq), H-Leu-NH₂ (153 g, 1.18 mol, 1.25 eq) (H-Leu-NH₂HCl salt has also been used), and 6-Cl-HOBT (183 g, 1.08 mol, 1.15 eq) were dissolved in DMF (18.3 L, 7 vol) treated with DIEA (287 ml, 1.65 mol, 1.75 eq) and stirred until dissolved while cooling to <2.5° C. TBTU (347 g, 1.08 mol, 1.15 eq) was slurried in DMF (1 vol, 2.6 L) and added and the solution which was stirred for 15 minutes at 0±5° C., and then allowed to react at 35±5° C. until the reaction was shown complete by HPLC.

The Fmoc chemical protecting group of the peptide fragment Fmoc-AA(27-38)-NH₂ was then removed prior to isolation of the fragment H-AA(27-38)-NH₂. Piperidine (372 mL, 3.76 mol, 4 eq) was added and the solution was stirred at 35±5° C. until analysis by HPLC showed that substantially all the Fmoc was removed from the peptide fragment. The solution was cooled to <10° C. and pre-cooled water (20.8 L. 8 vol) was slowly added to precipitate the product. The free-flowing slurry was aged at 10-15° C., and then isolated by filtration. The collected solid was washed with 25% EtOH/water and air dried. The peptide fragment was then reslurried in 25% EtOH/water (20.8 L, 8 vol) for >2 hours. The solids were collected, washed, and air dried. The wet solids were then reslurried in MTBE/heptane (1:1, 8 vol, 20.8 L), collected by filtration, washed with MTBE/heptane (2×4 vol, 10.4 L each) and air dried. A second MTBE/heptane reslurry was conducted after which the solids were dried in a vacuum oven at 35±5° C. The result was 2475 g (98.4%) of a preparation of isolated peptide fragment H-AA(27-38)-NH₂.

For the preparation of fragment Ac-AA(1-38)-NH₂ a solution phase reaction was performed in which peptide fragment H-AA(27-38)-NH₂ was combined with peptide fragment Ac-AA(1-26)-OH to yield Ac-(1-38)-NH₂. Peptide fragment Ac-AA(1-26)-OH (1.18 kg, 0.25 mol, 1.0 eq), peptide fragment H-AA(27-38)-NH₂ (836 g, 0.31 mol, 1.25 eq), 6-Cl-HOBT (57 g, 338 mmol, 1.35 eq) and DIEA (78 mL, 0.45 mol, 1.8 eq) were dissolved in DMF (8.9 L, 7.5 vol), and cooled to <2.5° C. Added to the reaction was TBTU (108 g, 338 mmol, 1.35 eq) slurried in DMF (0.5 vol, 0.6 L). The reaction was stirred for 15 min at 0±5° C. and at 30±5° C. until the reaction was shown to be complete by HPLC. The reactor was cooled, and pre-cooled water (9.4 L, 8 vol) was added with rapid stirring. The resulting solid was isolated by filtration and washed with additional water. The solid was reslurried in 20% ethanol/water (7.1 L, 6 vol), recollected by filtration and washed with water. The collected solid was dried in a vacuum oven at 35±5° C. The result was 2005 g (108%) of a preparation of fully protected, isolated HIV fusion inhibitor peptide Ac-AA(1-38)-NH₂.

As an aspect of the process for synthesis of crude SEQ ID NO:9 peptide, the side chain chemical protecting groups of HIV fusion inhibitor peptide Ac-AA(1-38)-NH₂ may be removed by acidolysis or any other method known to those skilled in the art for deprotecting a peptide by removing side chain chemical protecting groups. As one example, HIV fusion inhibitor peptide Ac-AA(1-38)-NH₂ (678 g, 92 mmol) was treated with TFA (trifluoracetic acid):DTT(dithiothreitol):water (90:15:5; 10 vol) and stirred at 20+/−2° C. for 5 hours. In a preferred embodiment, 10-15 eq DTT, rather than 5 eq, was used to optimize results. The solution was cooled to below 10° C., preferably below 5° C., and pre-cooled MTBE (25 vol, 17 L, <0° C., preferably <−15° C.) was slowly added at a rate such that the temperature remained below 10° C., preferably between 7 and 8° C. The slurry was aged at 10-15° C. and the resulting solids were collected by filtration, washed with MTBE, and air dried. The resulting powder was then slurried in acetonitrile (ACN; 10 vol, 6.8 L), the pH was adjusted to between 4 and 5, preferably closer to 5, with DIEA and acetic acid, and the slurry was stirred at 25±5° C. to decarboxylate the peptide. Once this was complete by HPLC, the solids were collected by filtration, washed with ACN, and dried to yield a preparation of deprotected and decarboxylated peptide (414 g, 100%).

In one embodiment, the 1144 peptide SEQ ID NO:9 was purified, concentrated and directly precipitated from a concentration column. SEQ ID NO:9 (270 g) was purified in three injections by RP-HPLC in aqueous NH₄OAc/acetonitrile buffer. The acceptable fractions were pooled and diluted by water until the total content of acetonitrile in the buffer was approximately 28%. The solution was reloaded onto the HPLC column and the peptide was eluted with 1:1 aqueous NaOAc/acetonitrile buffer. The pH of pooled fractions was adjusted to 5-6 and then total content of acetonitrile was brought up to above 85% by adding acetonitrile to the solution. The precipitated solid was collected by vacuum filtration, washed by 90% acetonitrile/water and dried in the vacuum oven to yield 78 g pure SEQ ID NO:9 peptide.

### Example 9

In one approach using a 2 fragment Rink-loaded CTC strategy, H-AA(27-38)-Rink-OH (table 1, sequence 27) was synthesized by standard solid phase synthesis with H at the N-terminus, and Rink Linker-OH (p-[(R,S)-α-amino-2,4-dimethoxybenzyl]-phenoxyacetic acid) at the C-terminus as a chemical protecting group.

A solution phase reaction was then performed in which peptide fragment H-AA(27-38)-Rink-OH (SEQ ID NO:18) was combined with peptide fragment Ac-AA(1-26)-OH (SEQ ID NO:10) to yield an HIV fusion inhibitor peptide having the amino acid sequence of SEQ ID NO:9. Peptide fragment Ac-AA(1-26)-OH (1.0 g, 0.21 mmol, 1.0 eq), 6-Cl-HOBT (39 mg, 0.23 mmol, 1.1 eq) and DIEA (55 μL, 0.32 mmol, 1.5 eq) were dissolved in DMF (10 ml, 10 vol), and cooled to 0-5° C. Added to the reaction was TBTU (71 mg, 0.22 mmol, 1.05 eq). The reaction was stirred for 15 minutes at 0-5° C. and peptide fragment H-AA(27-38)-Rink-OH (628 mg, 0.21 mmol, 1.0 eq), separately dissolved in DMF (2 mL) with DIEA (37 μL, 0.21 mmol, 1.0 eq) and cooled to 0-5° C., was added. The reaction was stirred for 15 min at 0-5° C. and at 25±5° C. for 3 hours or until the reaction was shown to be complete by HPLC. The reactor was cooled, and water (10 vol) was added with rapid stirring. The resulting solid was isolated by filtration and washed with additional water. The solid was reslurried in water/isopropanol, recollected by filtration and washed with water. The collected solid was dried in a vacuum oven at 35±5° C. The result was 1.55 g (95.7%) of a preparation of fully protected, isolated HIV fusion inhibitor peptide Ac-AA(1-38)-Rink-OH. The HIV fusion inhibitor peptide was then deprotected (by removing the side chain chemical protecting groups and the C-terminal Rink group) and decarboxylated (at the tryptophan residues) by using the methods described above, or any other method known to those skilled in the art, for deprotection and decarboxylation; and then purified (e.g., by HPLC) The result was a preparation (deprotected, decarboxylated, and purified) of HIV fusion inhibitor peptide having the SEQ ID NO:9 sequence (in this illustration, acetylated at the N-terminus, and amidated at the C-terminus).

### Example 10

In a further embodiment, using a 2 fragment approach with a super acid sensitive resin to generate the SEQ ID NO:9 peptide, a peptide fragment comprising amino acids 27-38 of SEQ ID NO:9 (“H-AA(27-38)-NH₂”) was synthesized by standard solid phase synthesis on Sieber amide resin (other amide resins such as Ramage could be used) with H at the N-terminus, and —NH₂ at the C-terminus.

A solution phase reaction was then performed in which peptide fragment H-AA(27-38)-NH₂ was combined with peptide fragment Ac-AA(1-26)-OH (SEQ ID NO:40) to yield an HIV fusion inhibitor peptide having the amino acid sequence of SEQ ID NO:9. Peptide fragment Ac-AA(1-26)-OH (1 g, 0.21 mmol, 1.0 eq), Peptide fragment H-AA(27-38)-NH₂ (677 mg, 0.25 mmol, 1.2 eq), 6-Cl-HOBT (39 mg, 0.23 mmol, 1.1 eq) and DIEA (92 μL, 0.053 mmol, 2.5 eq) were dissolved in DMF (10 ml, 10 vol), and cooled to 0-5° C. Added to the reaction was TBTU (75 mg, 0.23 mmol, 1.1 eq). The reaction was stirred for 15 minutes at 0-5° C. and then at 25±5° C. for 3 hours or until the reaction was shown to be complete by HPLC. The reactor was cooled, and water (10 vol) was added with rapid stirring. The resulting solid was isolated by filtration and washed with additional water. The solid was reslurried in water/isopropanol, recollected by filtration and washed with water. The collected solid was dried in a vacuum oven at 35±5° C. The result was 1.58 g (101%) of a preparation of fully protected, isolated HIV fusion inhibitor peptide Ac-AA(1-38)-NH₂. The HIV fusion inhibitor peptide was then deprotected (by removing the side chain chemical protecting groups and the C-terminal Rink group) and decarboxylated (at the tryptophan residues) by using the methods described above, or any other method known to those skilled in the art, for deprotection and decarboxylation; and then purified (e.g., by HPLC) The result was a preparation (deprotected, decarboxylated, and purified) of HIV fusion inhibitor peptide having SEQ ID NO:9 (in this illustration, acetylated at the N-terminus, and amidated at the C-terminus).

### Example 11

In a further preferred 2 fragment approach for synthesis of SEQ ID NO:9, a fragment comprising amino acids 27-38 of SEQ ID NO:9 (“AA(27-38)-NH₂”), was synthesized by standard solid phase synthesis starting from H-Glu-(CTC resin)-Leu-NH₂ (synthesis of this is described below) with H at the N-terminus, and —NH₂ at the C-terminus and with the side-chain of Glu₃₇ bound to CTC resin. (see, Table 7; also referred to herein as “H-AA(27-38)-NH₂ free Glu₃₇”).

A solution phase reaction was then performed in which peptide fragment H-AA(27-38)-NH₂ free Glu₃₇ side chain (SEQ ID NO:9) was combined with peptide fragment Ac-AA(1-26)-OH (SEQ ID NO:40) to yield an HIV fusion inhibitor peptide having the amino acid sequence of 291144 Peptide fragment Ac-AA(1-26)-OH (1.0 g, 0.21 mmol, 1.0 eq), 6-Cl-HOBT (39 mg, 0.23 mmol, 1.1 eq) and DIEA (55 μL, 0.32 mmol, 1.5 eq) were dissolved in DMF (10 ml, 10 vol), and cooled to 0±5° C. Added to the reaction was TBTU (71 mg, 0.22 mmol, 1.05 eq). The reaction was stirred for 15 minutes at 0±5° C. and peptide fragment H-AA(27-38)-NH₂, free Glu₃₇ (556 mg, 0.21 mmol, 1 eq), separately dissolved in DMF (2 mL) with DIEA (37 μL, 0.21 mmol, 1.0 eq) and cooled to 0-5° C., was added. The reaction was stirred for 15 min at 0±5° C. and at 25±5° C. for 3 hours or until the reaction was shown to be complete by HPLC. The reactor was cooled, and water (10 vol) was added with rapid stirring. The resulting solid was isolated by filtration and washed with additional water. The collected solid was reslurried in water/isopropanol, reisolated by filtration, washed with water, and dried in a vacuum oven at 35±5° C. The result was 1.43 g (92%) of a preparation of fully protected, isolated HIV fusion inhibitor peptide Ac-AA(1-38)-NH₂ free Glu₃₇ (SEQ ID NO:9), as determined by HPLC analysis for purity. The HIV fusion inhibitor peptide was then deprotected (by removing the side chain chemical protecting groups and the C-terminal Rink group) and decarboxylated (at the tryptophan residues) by using the methods described herein in Example 8, or any other method known to those skilled in the art, for deprotection and decarboxylation; and then purified (e.g., by HPLC) The result was a preparation (deprotected, decarboxylated, and purified) of HIV fusion inhibitor peptide SEQ ID NO:9 (in this illustration, acetylated at the N-terminus, and amidated at the C-terminus).

### Example 12

Illustrated herein is a method for synthesis of an HIV fusion inhibitor peptide having the 1144 amino acid sequence (SEQ ID NO:9) using a linear solid phase synthesis approach. An HIV fusion inhibitor peptide with SEQ ID NO:9 can be synthesized on solid phase support in linear fashions in high yield and high purity, which requires only one loaded resin as starting material, and does not involve solution condensation of fragments and deprotection of corresponding fragment in solution phase as part of simplifying the method for synthesis.

A SEQ ID NO:9 peptide was synthesized by standard solid phase synthesis (using amide resins, e.g., Sieber resin, or Rink resin (others, such as Ramage, are also available); or modified acid resins, e.g., rink-linker-loaded CTC resin, or gamma-glutamyl(Leu-amide)-loaded CTC resin) with acetylation (“Ac”, as a chemical group) of the N-terminus, while having a amide group (—NH₂) at the C-terminus. The peptide was cleaved from the resin used for its solid phase synthesis and the side chain chemical protecting groups of HIV fusion inhibitor peptide Ac-AA(1-38)-NH₂ may be removed by acidolysis or any other method commonly known to those skilled in the art for deprotecting a peptide by removing side chain chemical protecting groups. The deprotected and decarboxylated peptide was then purified by HPLC or other suitable chromatographic techniques to yield a preparation of isolated HIV fusion inhibitor SEQ ID NO:9 peptide.

The rink-linker-loaded CTC resin can be obtained by loading Fmoc-rink linker onto CTC resin by methods known to those skilled in the art. In this example, Fmoc-gamma-glutamyl(Leu-amide) and its loading to CTC resin is illustrated. Fmoc-Glu(OtBu)-OH (22.175 g, 50 mmol), 6-Cl-HOBt (10.176 g, 60 mmol), DIEA (21.78 ml, 125 mmol) and H-Leu-NH2 (9.764 g, 75 mmol) were dissolved in DMF (220 mL, 10 vol) and cooled to below 5° C., then TBTU (19.266 g, 60 mol) was added into the reaction flask. The reaction mixture was stirred for 30 min at below 5° C. and 1 hr at room temperature or until HPLC showed the completion of reaction. Fmoc-Glu(OtBu)-Leu-NH₂ was precipitated by water, washed by 0.2% N HCl and reslurried in 20% IPA and 4% NaHCO₃. After removal of t-Bu protection group by treating with 95% TFA/water, Fmoc-Glu-Leu-NH₂ was loaded onto CTC resin by the method known to those skilled in the art to provide gamma-glutamyl(Leu-amide)-loaded CTC resin.

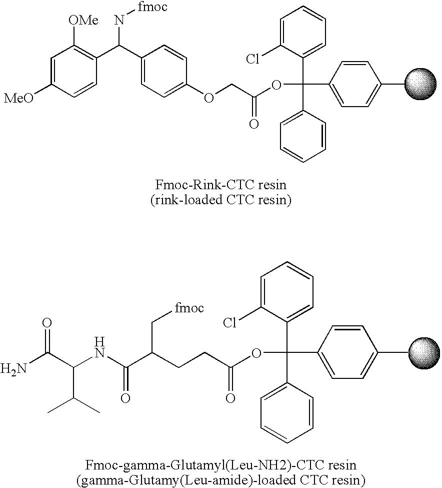

### Example 13

In an approach to the isolation and purification of the SEQ ID NO:9 peptide, pure lyophilized Ac-AA(1-38)-NH₂ (908 mg) was slurried in 8:2 Acetonitrile:water (18 mL, 20 vol) (10-30 volume acceptable) for 3 hours at 20±5° C. (1-5 hours acceptable). The product was reisolated by filtration, washed with 8:2 Acetonitrile:water, and dried under vacuum at 35±5° C. to yield 854 mg (94%) pure, desalted Ac-AA(1-38)-NH₂.

Significant reduction in the salt content of SEQ ID NO:9 (such that it is acceptable for formulation) may also be accomplished by loading pure SEQ ID NO:9 onto a Sephadex column and eluting with 9:1 water/ACN or by reloading back onto a RP-HPLC column and eluting with 5 mM NH₄OAc/ACN 1:1. In both cases the peptide is reisolated by lyophilization.

Precipitation 1 of lyophilized SEQ ID NO:9 peptide. 1144 (SEQ ID NO:9) solid (186.2 g, previously isolated by lyophilization) was dissolved in 9312 mL 20% ACN in water and the pH lowered to ˜1.6 with TFA (46 mL). Then the pH was increased with 1 N NaOH (583 mL) to a final pH of 4.76. The flask was cooled in an ice-bath for 2 hours and then the solids were filtered off, and dried in a vacuum oven at 35±5° C., affording 179.5 g (96%) pure, desalted Ac-AA(1-38)-NH₂.

Precipitation 2 of lyophilized SEQ ID NO:9 peptide. 1144 (SEQ ID NO:9) solid (previously isolated by lyophilization) was dissolved in 50% acetonitrile-water at concentration of 50 mg/ml by adjusting pH to 8-9. Then pH of the solution was adjusted back to 5-6 before adding more acetonitrile to bring total content of acetonitrile is above 85%. The precipitated solid was collected by vacuum filtration, washed by 90% acetonitrile/water and dried in the vacuum oven.

In another embodiment, 1144 (SEQ ID NO:9) solid (previously isolated by lyophilization) was dissolved in 50% acetonitrile/water (14 vol) at 70 mg/ml concentration with pH adjustment by NH₄OH to 6-9. After a polish filtration, the solution was added into acetonitrile (48 vol) and 2 vol more of 50% acetonitrile/water was used to wash the dissolution tank and lines. The resulting slurry was stirred for 10 min before the vacuum filtration. 10 vol of 90% acetonitrile/water and straight acetonitrile (10 vol each) were used to wash the solid. The collected peptide was dried in vacuum oven until at constant weight.

In another embodiment, pure 1144 (SEQ ID NO:9) solution in 50% ACN, 50% 10 mM NaOAC was acidified to pH 5-6 and diluted with acetonitrile to precipitate the product. The resulting solids were isolate by filtration and washed with 90% acetonitrile/water and then straight acetonitrile (10 vol each). The collected peptide was dried in a vacuum oven until at constant weight.

### Example 14

Further provided herein are methods for administering an antiviral peptide, e.g., and HIV fusion inhibitor peptide, itself or as an active drug substance in a composition provided herein, in treatment of, therapy for, or as part of a therapeutic regimen for, HIV infection and/or AIDS. Antiviral activity of an HIV fusion inhibitor peptide can be utilized in a method for inhibiting transmission of HIV to a target cell, comprising contacting the virus and/or cell with an amount of HIV fusion inhibitor peptide effective to inhibit infection of the cell by HIV, and more preferably, to inhibit HIV-mediated fusion between the virus and the target cell. This method can be used to treat HIV-infected patients (therapeutically) or to treat patients newly exposed to or at high risk of exposure (e.g., through drug usage or high risk sexual behavior) to HIV (prophylactically). Thus, for example, in the case of an HIV-1 infected patient, an effective amount of HIV fusion inhibitor peptide would be a dose sufficient (by itself and/or in conjunction with a regimen of doses) to reduce HIV viral load in the patient being treated. As known to those skilled in the art, there are several standard methods for measuring HIV viral load which include, but are not limited to, by quantitative cultures of peripheral blood mononuclear cells and by plasma HIV RNA measurements. The HIV fusion inhibitor peptides can be administered in a single administration, intermittently, periodically, or continuously, as can be determined by a medical practitioner, such as by monitoring viral load. Depending on the particular composition provided herein containing the HIV fusion inhibitor peptide, and such factors as whether or not the particular composition provided herein is further comprising a pharmaceutically acceptable carrier and/or macromolecular carrier, the HIV fusion inhibitor peptide can be administered with a periodicity ranging from days to weeks or possibly longer. Further, an HIV fusion inhibitor peptide can be used, in antiviral therapy, when used in combination or in a therapeutic regimen (e.g., when used simultaneously, or in a cycling on with one drug and cycling off with another) with other antiviral drugs or prophylactic agents used for treatment of HIV.

One commonly used treatment involving a combination of antiviral agents, is known as HAART (Highly Active Anti-Retroviral Therapy). HAART typically combines three or more drugs with antiviral activity against HIV, and typically involves more than one class of drug (a “class” referring to the mechanism of action, or viral protein or process targeted by the drug). Thus, a composition provided herein containing an HIV fusion inhibitor peptide can be administered alone (e.g., as monotherapy) or can be administered in a treatment regimen, or co-administered, involving a combination of additional therapeutic agents for the treatment of HIV infection and/or AIDS, as described in more detail herein.

For example, in one embodiment, one or more therapeutic agents can be combined in treatment with an HIV fusion inhibitor peptide in a composition provided herein. Such a combination can comprise at least one antiviral agent in addition to the HIV fusion inhibitor peptide. Such combinations can, for example, be prepared from effective amounts of antiviral agents (useful in treating of HIV infection) currently approved or approved in the future, which include, but are not limited to, one or more additional therapeutic agents selected from the following: antiviral agents such as cytokines, e.g., rIFN α, rIFN β, rIFN γ; reverse transcriptase inhibitors, including but not limited to, abacavir, AZT (zidovudine), ddC (zalcitabine), nevirapine, ddI (didanosine), FTC (emtricitabine), (+) and (−) FTC, reverset, 3TC (lamivudine), GS 840, GW-1592, GW-8248, GW-5634, HBY097, delaviridine, efavirenz, d4T (stavudine), FLT, TMC125, adefovir, tenofovir, and alovudine; protease inhibitors, including but not limited to, amprenivir, CGP-73547, CGP-61755, DMP-450, indinavir, nelfinavir, PNU-140690, ritonavir, saquinavir, telinavir, tipranovir, atazanavir, lopinavir, ABT378, ABT538 and MK639; inhibitors of viral mRNA capping, such as ribavirin; amphotericin B as a lipid-binding molecule with anti-HIV activity; castanospermine as an inhibitor of glycoprotein processing; viral entry inhibitors such as fusion inhibitors (enfuvirtide, T1249, other fusion inhibitor peptides, and small molecules), SCH-D, UK-427857 (Pfizer), TNX-355 (Tanox Inc.), AMD-070 (AnorMED), Pro 140, Pro 542 (Progenics), FP-21399 (EMD Lexigen), BMS806, BMS-488043 (Bristol-Myers Squibb), maraviroc (UK-427857), ONO-4128, GW-873140, AMD-887, CMPD-167, and GSK-873,140 (GlaxoSmithKline); CXCR4 antagonist, such as AMD-070); lipid and/or cholesterol interaction modulators, such as procaine hydrochloride (SP-01 and SP-01A); integrase inhibitors, including but not limited to, L-870, and 810; RNAseH inhibitors; inhibitors of rev or REV; inhibitors of vif (e.g., vif-derived proline-enriched peptide, HIV-1 protease N-terminal-derived peptide); viral processing inhibitors, including but not limited to betulin, and dihydrobetulin derivatives (e.g., PA-457); and immunomodulators, including but not limited to, AS-101, granulocyte macrophage colony stimulating factor, IL-2, valproic acid, and thymopentin. As appreciated by one skilled in the art of treatment of HIV infection and/or AIDS, a combination drug treatment can comprise two or more therapeutic agents having the same mechanism of action, or can comprise two or more therapeutic agents having a different mechanism of action.

Effective dosages of these illustrative additional therapeutic agents, which can be used in combination with an HIV fusion inhibitor peptide, and/or a composition provided herein, are known in the art. Moreover, effective dosages of an HIV fusion inhibitor peptide or pharmaceutical composition provided herein to be administered can be determined through procedures well known to those in the art; e.g., by determining potency, biological half-life, bioavailability, and toxicity. In one embodiment, an effective amount of an HIV fusion inhibitor peptide and its dosage range are determined by one skilled in the art using data from routine in vitro and in vivo studies well know to those skilled in the art. For example, in vitro infectivity assays of antiviral activity, such as described herein, enables one skilled in the art to determine the mean inhibitory concentration (IC) of the compound, as the sole active ingredient or in combination with other active ingredients, necessary to inhibit a predetermined range of viral infectivity (e.g., 50% inhibition, IC₅₀; or 90% inhibition, IC₉₀) or viral replication. Appropriate doses can then be selected by one skilled in the art using pharmacokinetic data from one or more standard models, so that a minimum plasma concentration (C[min]) of the active ingredient is obtained which is equal to or exceeds a predetermined value for inhibition of viral infectivity or viral replication. While dosage ranges typically depend on the route of administration chosen and the formulation of the dosage, when administered, such as routes of administration including but not limited to, subcutaneously, parenterally, intradermal or orally, an exemplary dosage range of a compound, as an active ingredient, can be from about 1 mg/kg body weight to about 100 mg/kg body weight; and more preferably no less than 1 mg/kg body weight to no more than 10 mg/kg body weight. In one embodiment, administration is by injection (using, e.g., subcutaneous), In one embodiment, an HIV fusion inhibitor peptide.

Thus, there is provided a method for inhibition of transmission of HIV to a cell, comprising administering a composition described herein comprising an HIV fusion inhibitor peptide in an effective amount to inhibit infection of the cell by HIV. The method can further include administering a composition described herein in combination with other therapeutic agents used to treat HIV infection and/or AIDS to a patient by administering to the individual the combination (simultaneously or sequentially, or a part of a therapeutic regimen) of therapeutic agents which includes an effective amount of the HIV fusion inhibitor peptide or pharmaceutical composition provided herein. Also provided is a method for inhibiting HIV entry comprising administering to a patient in need thereof a composition described herein comprising an HIV fusion inhibitor peptide in an effective amount to inhibit viral entry of a target cell. The method may further comprise administering a composition described herein in combination with an effective amount of one or more additional inhibitors, e.g., inhibitors of viral entry, useful in treating HIV infection.

### Example 15

Methods for the preparation of compositions provided herein are set forth below. In addition, illustrative compositions are described.

Materials: Sucrose acetate isobutyrate (SAIB) was obtained from Eastman Chemicals. Polylactide (PLA) and Polylactide-co-glycolide (PLGA) were obtained from Lakeshore Biomateials. PLA and PLGA differ in lactide:glycolide ratio, molecular weight and their endgroup. All PLGAs used in this Study were 50:50 lactide:glycolide. Molecular weight is graded by the number in the name. An estimate of the molecular weight is 10000 times the number. The endgroup is either carboxylic acid (A), methyl ester (M) or lauryl ester (L). N-methyl-2-pyrrolidone (NMP) was obtained from Spectrum. Benzylbenzoate and triacetin were obtained from Sigma. GuanidineHCl was obtained from Amresco. Tris-HCl was obtained from Sigma. 4-(2-pyridylazo)resorcinol was obtained from Sigma. Methanol was obtained from VWR. Zinc Sulfate Heptahydrate was obtained from Sigma. Zinc Chloride was obtained from Sigma.

Preparation of T1144 Peptide Material: T1144 peptide material was prepared according to the following protocols.

Spray drying: T1144 peptide was dissolved at a pH less than 4 or greater than 6, usually in water. 1N NaOH or 1N HCl were used to adjust pH. Peptide solution was sprayed through an atomizing nozzle into a heated chamber. Dried peptide particles were collected manually.

Peptides can further be prepared by the spray drying method described above, but with an excipient added to the spray drying solution, thereby incorporating the excipient and the peptide.

Salt or pH precipitation: Peptide was dissolved at a pH less than 4 or greater than 6, usually in water. 1N NaOH or 1N HCl were used to adjust pH. Either a salt solution or strong acid/base was added to cause precipitation.

Precipitate was collected by centrifugation, dried by lyophilization and passed through a 200 μm screen to control particle size.

Vehicle Preparation: Vehicles were prepared according to the following protocols.

SAIB Vehicle Preparation: an appropriate amount of SAIB to arrive at the desired final concentration was warmed and added to NMP, and mixed until uniform.

SAIB/PLA Vehicle Preparation: an appropriate amount of PLA to arrive at the desired final concentration was dissolved into NMP, benzylbenzoate or triacetin. An appropriate amount of SAIB to arrive at the desired final concentration was warmed and added to the PLA solution, and mixed until uniform.

PLA, PLG and PLGA Vehicle Preparation: an appropriate amount of PLA, PLG or PLGA to arrive at the desired final concentration of PLA, PLG or PLGA was dissolved into NMP.

Compositions can be prepared by any method known to those skilled in the art. In the present examples, peptide material (precipitated or spray-dried) was added to a vial. Vehicle was added to the vial, and the contents were mixed until uniform. In some cases, this required warming to ˜40° C. to ensure proper mixing. Formulas were quantified as peptide weight per formula weight in mg/g.

Peptide content was determined based on tryptophan and tyrosine absorbance in a manner similar to the Edelhoch method. Briefly, ˜1 mg peptide material was dissolved in 1 mL 8M guanidine hydrochloride. The solution was evaluated for UV absorbance at 276, 280 and 288 nm. Using these measured absorbance values, along with the known sample weight, sample volume, number of tryptophan and tyrosine residues in the peptide, and peptide molecular weight, peptide content (% w/w) of the solid was determined.

Metal cation content was determined using a UV/vis absorbance assay that employed the use of 4-(2-pyridylazo)resorcinol (PAR), a metallochromic indicator that is known to form a 2:1 complex with M²⁺. Briefly, ˜1 mg solid was dissolved in 1 mL of Tris-HCl buffer (pH 8) containing 6M guanidineHCl. This solution was diluted (using the same buffer) such that the final metal cation concentration was 1-10 μM. 50 μL of 0.1M PAR was added to 950 μL of the diluted solution. After equilibration, the test solution was evaluated for UV/vis absorbance at 500 nm. Metal cation concentration was calculated based on the linear least-squares analysis from a standard curve that was obtained on the same day, and the metal cation content (wt %) of the sample was determined.

Peptide concentration in plasma was determined by LC-MS evaluation. Plasma samples were diluted with 3 volumes of acetonitrile containing 0.5% (v/v) formic acid, centrifuged, and the supernatant assayed directly. Chromatography was performed using gradient elution (10 mM Ammonium acetate, pH6.8: acetonitrile, 0.6 mL/min) in a 6 minute total run time. Separations were performed on a Phenomenex Luna C8(2) 50×2 mm column protected by a 4×2 mm Phenomenex SecurityGuard C8 guard column. Mass spectrometry (ESI+) was performed on either Sciex API4000 or API4000 Qtrap instruments, usually in single-quad mode, with the [M+3H]³⁺ or [M+4H]⁴⁺ ions detected. Calibration curves for T1144 were constructed from 30 ng/mL to 30 ng/mL. Results are presented as plasma peptide concentration over time. The following abbreviations are used: Cₘₐₓ=maximum plasma peptide concentration; tₘₐₓ=time at Cₘₐₓ; t_{0.1}=time at which plasma peptide concentration drops below 0.1 μg/ml; and t_{0.01} is the time normalized plasma concentration drops below 0.01 μg/mL. In some cases, plasma concentrations were normalized to 3 mg peptide per kg animal weight to facilitate comparison.

Animals were dosed as follows. Excess T1144-containing composition was drawn into a 1 cc syringe through a 16G needle. This needle was replaced by an 18G or 21G needle, and the syringe emptied to the correct dose. Animals were dosed in the subcutaneous space between the scapulas. Rats (400 g) were dosed at 400 μL, three animals per dose group. Cynomolgus monkeys (2.5 kg) were dosed at either 400 μL or 1000 μL, three animals per group.

All pharmacokinetic data was collected using rats or monkeys. In some cases, plasma concentrations were normalized to 3 mg peptide/kg animal to facilitate comparison.

The following peptide containing compositions were prepared.

T1144/Zinc Precipitate A. T1144 was dissolved in water. pH was adjusted to ˜6.2 and water added to a concentration of 25 mg/mL. The solution was passed through a 0.22 μm filter. 2 mL 0.1M ZnSO4 was added to 80 mL 11144 solution. The resulting suspension was centrifuged, the supernatant decanted, and the precipitate frozen. The precipitate was lyophilized and passed through a 200 μm screen.

T1144/Zinc Precipitate B. T1144 was dissolved in water. pH was adjusted to ˜6.2 and water added to a concentration of 25 mg/mL. The solution was passed through a 0.22 μm filter. 60 mL 0.1M ZnSO₄ was added to 120 mL T1144 solution. The resulting suspension was centrifuged, the supernatant decanted, and the precipitate frozen. The precipitate was lyophilized and passed through a 200 μm screen.

T1144/Zinc Precipitate C. T1144 was dissolved in water. pH was adjusted to ˜5.7 and water added to a concentration of 40 mg/mL. The solution was passed through a 0.22 μm filter. <100 mg ZnCl₂ was added to 20 mL T1144 solution. The resulting suspension was centrifuged, the supernatant decanted, and the precipitate washed with 1 mL water. The precipitate was frozen, lyophilized and passed through a 200 μm screen.

T1144/Zinc Precipitate D. Precipitate B was washed with 5 mL water, centrifuged and the supernatant decanted. This was repeated twice more. The resulting precipitate was frozen, lyophilized and passed through a 200 μm screen.

T1144/Zinc Precipitate E. 445 mg ZnSO₄*7H₂O was dissolved in 2 mL water. 1.0 g Precipitate D was slurried in the zinc solution. The slurry was frozen, lyophilized and passed through a 200 μm screen.

T1144 Precipitate F. T1144 was dissolved in water. pH was adjusted to ˜6.2 and water added to a concentration of 25 mg/mL. The solution was passed through a 0.22 μm filter. 5 mL 1N Acetic Acid was added, decreasing pH to ˜5. The resulting suspension was centrifuged, the supernatant decanted, and the precipitate frozen. The precipitate was lyophilized and passed through a 200 μm screen.

T1144/Zinc Precipitate G. 230 mg ZnSO₄*7H₂O was dissolved in 2 mL water. 500 mg Precipitate F was slurried in the zinc solution. The slurry was frozen, lyophilized and passed through a 200 μm screen.

T1144/Zinc Precipitate H. T1144 was dissolved in water. pH was adjusted to ˜8.4 and water added to a concentration of 50 mg/mL. The solution was passed through a 0.22 μm filter. Methanol was added to a concentration of 25 mg/mL (50:50 Methanol:Water). ˜1 mL 0.1M ZnSO₄ was added to 20 mL TRI-1144 solution. The resulting suspension was centrifuged, the supernatant decanted, and the precipitate frozen. The precipitate was lyophilized and passed through a 200 μm screen.

T1144 Precipitate I. TRI-1144 was dissolved in water. pH was adjusted to ˜8.4 and water added to a concentration of 50 mg/mL. The solution was passed through a 0.22 μm filter. Methanol was added to a concentration of 25 mg/mL (50:50 Methanol:Water). pH was adjusted to ˜5. The resulting suspension was centrifuged, the supernatant decanted, and the precipitate frozen. The precipitate was lyophilized and passed through a 200 μm screen.

T1144/Zinc Precipitate J. T1144 was dissolved in water. pH was adjusted to ˜6.2 and water added to a concentration of 25 mg/mL. The solution was passed through a 0.22 μm filter. 60 mL 0.1M ZnSO4 was added to 120 mL T1144 solution. The resulting suspension was centrifuged, the supernatant decanted, and the precipitate frozen. The precipitate was lyophilized and passed through a 150 μm screen.

T1144/Zinc Precipitate K. T1144 was dissolved in water. pH was adjusted to ˜6.2 and water added to a concentration of 25 mg/mL. The solution was passed through a 0.22 μm filter. 1 mL 0.1M ZnSO4 was added to 40 mL T1144 solution. The resulting suspension was centrifuged, the supernatant decanted, and the precipitate frozen. The precipitate was lyophilized and passed through a 150 μm screen.

T1144/Zinc Precipitate L. 1.0 g Precipitate J was washed with 30 mL water, centrifuged and the supernatant decanted. This was repeated. The resulting precipitate was frozen, lyophilized and passed through a 200 μm screen.

T1144/Zinc Precipitate M. T1144 was dissolved in water. pH was adjusted to ˜6.3 and water added to a concentration of 25 mg/mL. The solution was passed through a 0.22 μm filter. 25 mL T1144 solution was sprayed through an atomizing nozzle (in a manner similar to spray drying) into 50 mL of a vigorously-mixed 0.3M ZnSO4 solution. The resulting suspension was centrifuged, the supernatant decanted, and the precipitate frozen. The precipitate was lyophilized and passed through a 200 μm screen.

T1144/Zinc Precipitate N. T1144 was dissolved in water. pH was adjusted to ˜6.3 and water added to a concentration of 50 mg/mL. Methanol was added to a final T1144 solution concentration of 25 mg/mL. The solution was passed through a 0.22 μm filter. 25 mL T1144 solution was sprayed through an atomizing nozzle (in a manner similar to spray drying) into 50 mL of a vigorously-mixed 0.1M ZnSO4 solution. The resulting suspension was centrifuged and the supernatant decanted. The precipitate was washed with 10 mL water three times. The suspension was centrifuged, the supernatant decanted and the precipitate frozen. The precipitate was lyophilized and passed through a 200 μm screen.

TABLE 10
Peptide Material composition

| Peptide Material | Peptide content (%) | Zinc Content (%) |
|---|---|---|
| PRECIPITATED A | 90.6 | 1.6 |
| PRECIPITATED B | 72.5 | 7.8 |
| PRECIPITATED C | 87.7 | 1-5 |
| PRECIPITATED D | 89.3, 87.9 | 2.9, 2.4 |
| PRECIPITATED E | 70.1 | 10.0 |
| PRECIPITATED F | 88.3 | N/A |
| PRECIPITATED G | 71.7 | 7.7 |
| PRECIPITATED H | 90.6, 89.7 | 1.7, 1.9 |
| PRECIPITATED I | 87.7 | N/A |
| PRECIPITATED J | 60.2 | 11.5 |
| PRECIPITATED K | 88.2 | 1.7 |
| PRECIPITATED L | 93.7 | 1.9 |
| PRECIPITATED M | 60.9 | 12.4 |
| PRECIPITATED N | 92.5 | 2.1 |

Compositions described above were administered to rats or monkeys as described below. The results obtained in rats or monkeys are expected to reasonably correlate with human results.

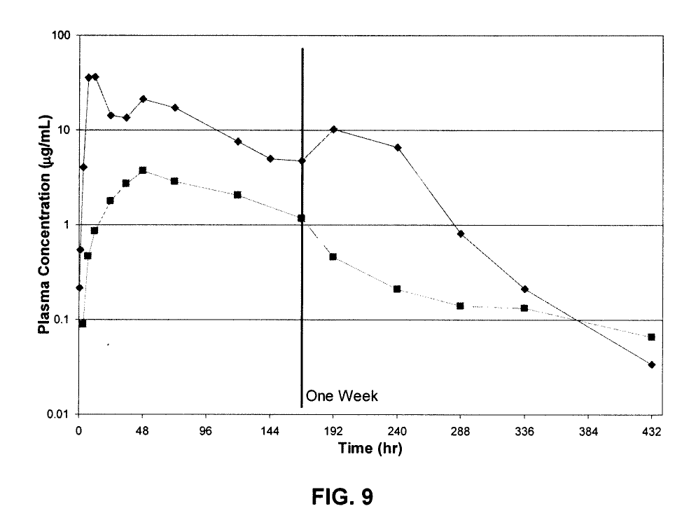

Precipitate D was formulated at 100 mg/g in 74:11:15 SAIB:PLA3L:NMP and dosed at 1000 μL in cynomolgus monkeys. As shown in FIG. 9 (--♦--), plasma concentration was greater than the target value of 1 μg/mL for 12 days, exceeding the target time of 7 days. Precipitate J was formulated at 50 mg/g in 40:60 PLA3L:NMP and dosed at 400 μL in cynomolgus monkeys. As shown in FIG. 9 (--▪--), plasma concentration was greater than the target value of 1 μg/mL for 7 days; a larger dose, such as 1000 μL of 100 mg/g Precipitate J, potentially could have yielded target plasma concentrations for 10-12 days. This indicates that T1144 can be delivered subcutaneously in either a SAIB/PLA or PLA vehicle and provide plasma concentrations in excess of target values (i.e., 1 μg/mL) for greater than one week.

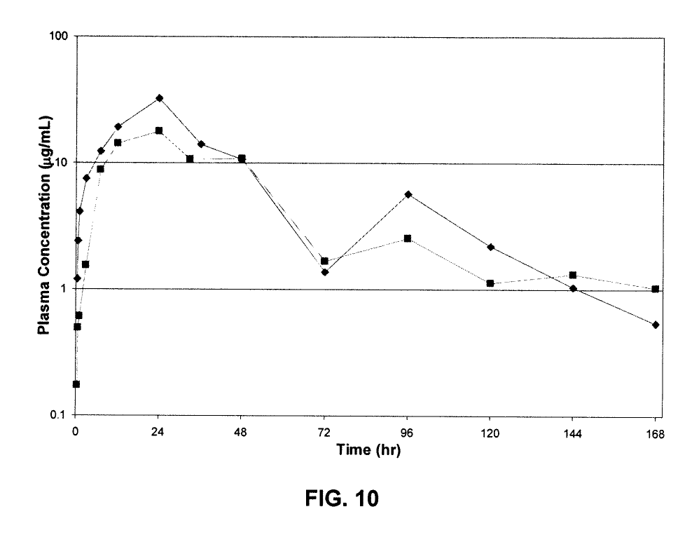

Precipitate D was formulated at 100 mg/g in 74:11:15 SAIB:PLA3L:NMP and dosed at 400 μL in rats. As shown in FIG. 10 (--♦--), plasma concentration was greater than the target value of 1 μg/mL for 6 days, nearly meeting the target time of 7 days. Precipitate J was formulated at 50 mg/g in 40:60 PLA3L:NMP and dosed at 400 μL in rats. As shown in FIG. 10 (--▪--), plasma concentration was greater than the target value of 1 μg/mL for 7 days. This indicates the formulations provided similar sustained delivery of T1144 in both rodent and primate models.

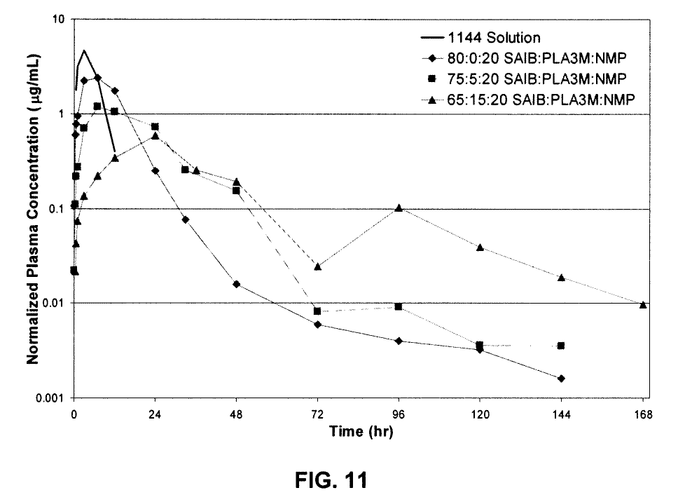

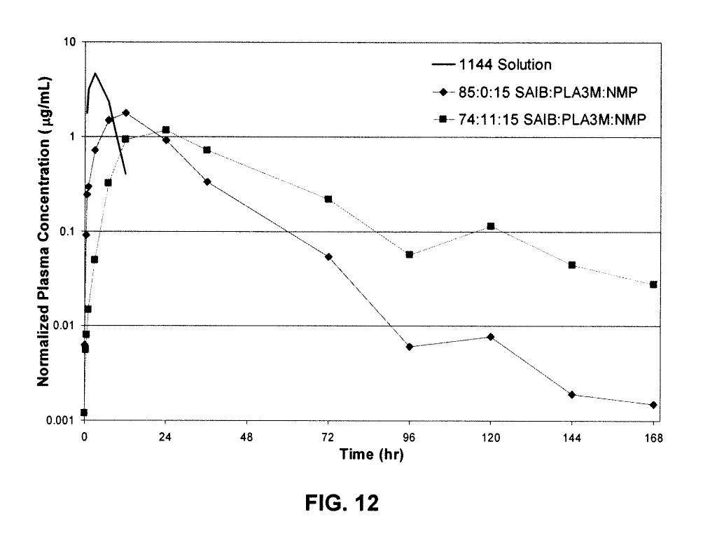

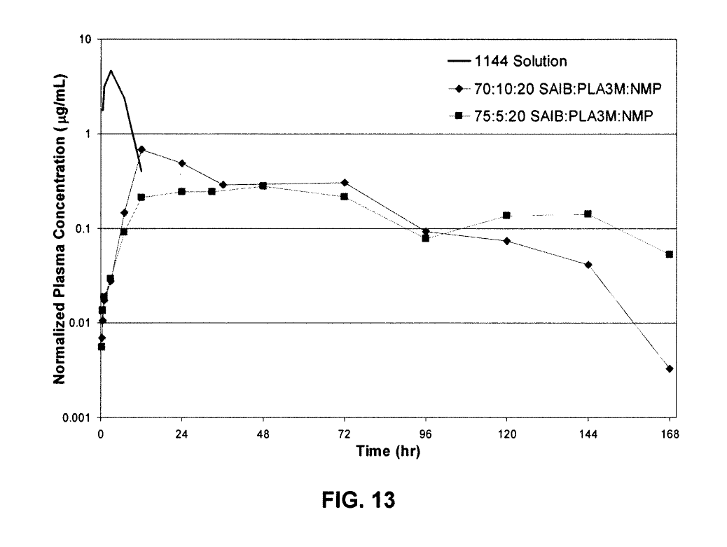

Influence of SAIB:PLA Ratio on Pharmacokinetic Profiles in Rat. Precipitate A was formulated at 50 mg/g in SAIB:PLA3M:NMP vehicles and dosed at 400 μL in rats. As shown in FIG. 11, decreasing the SAIB:PLA ratio (i.e., more PLA), decreased C_{MAX}, increased t_{MAX} and increased t_{0.01}. Precipitate B was also formulated at 50 mg/g in SAIB:PLA3M:NMP vehicles and dosed at 400 μL in rats. As shown in FIGS. 12 and 13, results were qualitatively similar to those presented for Precipitate A. However, the degree to which SAIB:PLA ratio influenced delivery from Precipitate B was less that its influence on delivery from Precipitate A. This indicates that decreasing the SAIB:PLA ratio improves sustained delivery of TRI-1144, and that precipitate properties, particularly the amount of zinc in the precipitate, can affect delivery rate.

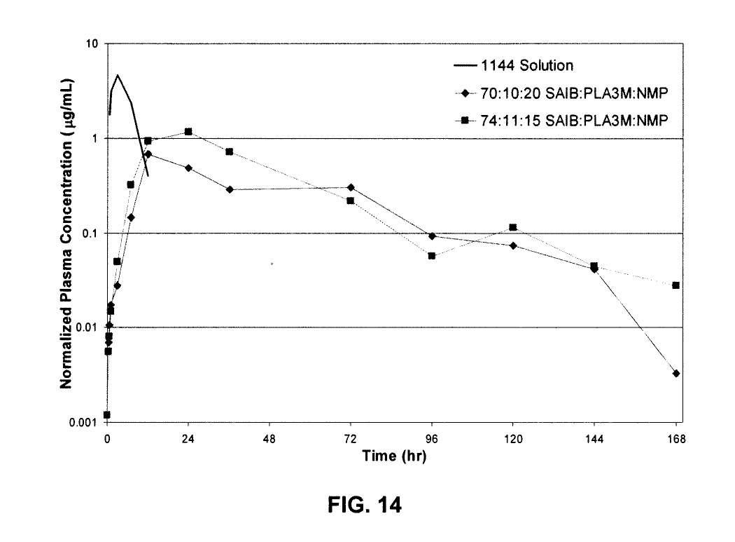

Influence of Matrix:Solvent Ratio on Pharmacokinetic Profiles in Rat. Precipitate B was formulated at 50 mg/g in SAIB:PLA3M:NMP vehicles and dosed at 400 μL in rats. As shown in FIG. 14, PLA levels around 10% can slow peptide delivery relative to lower PLA levels.

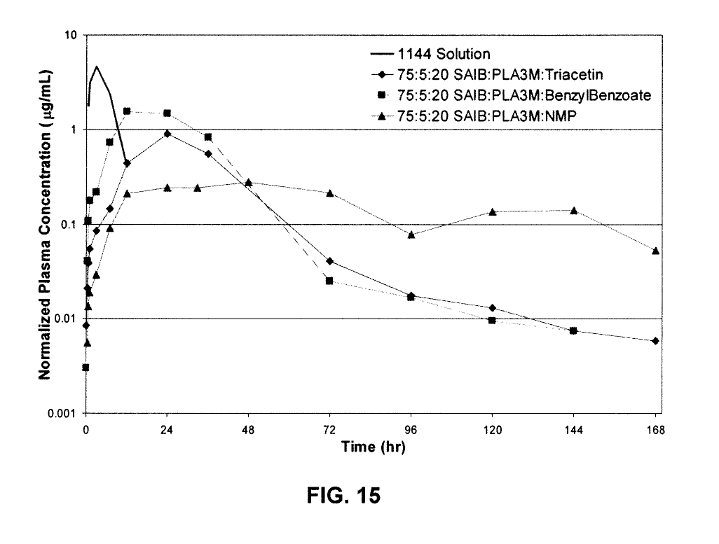

Influence of Solvent Type on Pharmacokinetic Profiles in Rat. Precipitate B was formulated at 50 mg/g in 75:5:20 SAIB:PLA3M:Solvent (i.e., triacetin, benzylbenzoate or NMP) vehicles and dosed at 400 μL in rats. As shown in FIG. 15, as solvent type was changed, C_{MAX} decreased, t_{MAX} increased and t_{0.01} increased. NMP gave more desirable pharmacokinetic properties than triacetin, which performed better than benzylbenzoate. This indicates that solvent type influences peptide delivery.

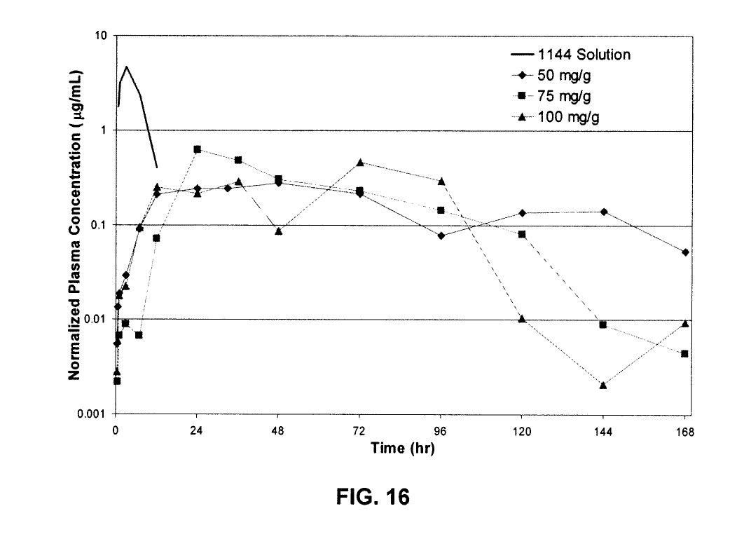

Influence of Peptide Concentration on Pharmacokinetic Profiles in Rat. Precipitate B was formulated in a 75:5:20 SAIB:PLA3M:NMP vehicle and dosed at 400 μL in rats. The results are shown in FIG. 16, and indicate that, in this vehicle, peptide concentration can be increased without adversely affecting peptide delivery.

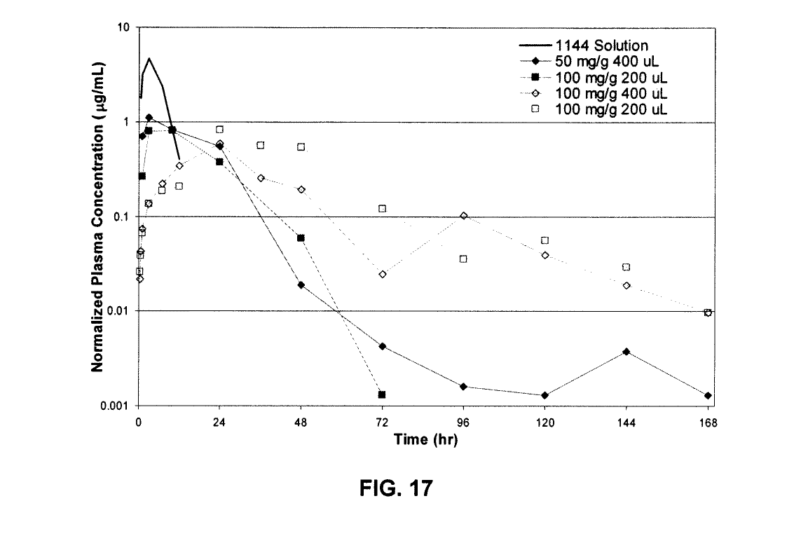

Influence of Injection Volume on Pharmacokinetic Profiles in Rat. Precipitate B was formulated at 100 mg/g in a 74:11:15 SAIB:PLA3M:NMP vehicle and dosed in rats. As shown in FIG. 17, there was no significant influence of dose volume on sustained delivery parameters; however, the 400 μL dose performed somewhat better than the 200 μL dose (all normalized). Precipitate C was formulated in a 77:15:8 SAIB:NMP:Ethanol vehicle and dosed in rats. As shown in FIG. 17, there was no significant influence of dose volume on sustained delivery parameters in the first three days when controlling peptide dose; however, the 400 μL dose performed somewhat better than the 200 μL dose after three days. This indicates that increasing injection volume can promote sustained delivery.

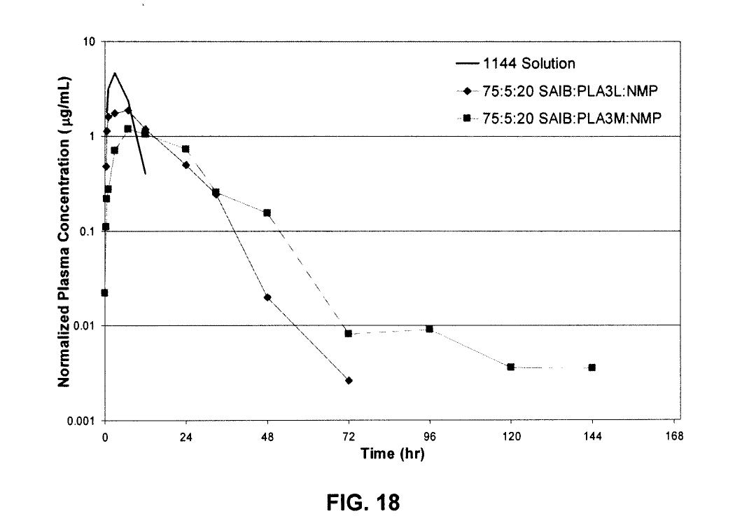

Influence of PLA Type in SAIB Vehicle on Pharmacokinetic Profiles in Rat. Precipitate A was formulated at 50 mg/g in 75:5:20 SAIB:PLA:NMP vehicles and dosed at 400 μL in rats. As shown in FIG. 18, changing PLA type from 3L to 3M decreased C_{MAX}, increased t_{MAX} and increased t_{0.01}. This indicates that PLA type can influence sustained delivery.

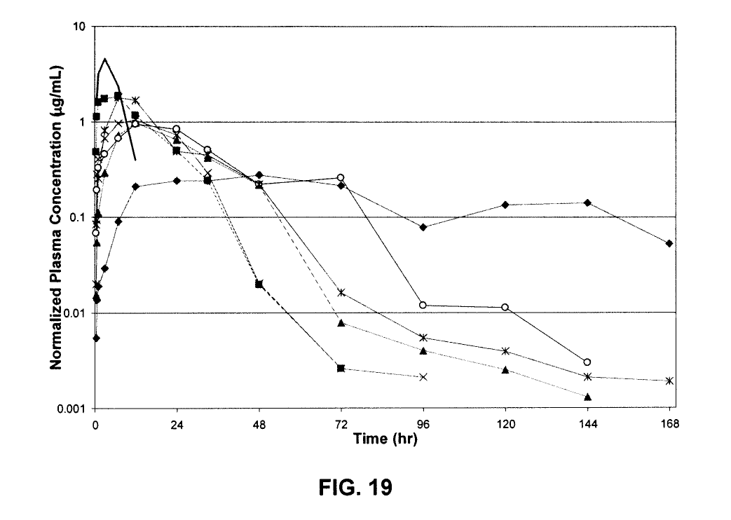

Influence of Peptide Precipitate Form on Pharmacokinetic Profiles in Rat. Precipitates A, B, E and G were formulated separately at 50 mg/g in a 75:5:20 SAIB:PLA3M:NMP vehicle and dosed at 400 μL in rats. As shown in FIG. 19, increasing zinc content during the initial precipitation process decreased C_{MAX}, increased t_{MAX} and increased t_{0.01} (A and B). Adding zinc sulfate as a lyophilized salt to a low-zinc precipitate decreased C_{MAX}, increased t_{MAX} and increased t_{0.01} (A and E). Precipitating with zinc instead of pH did not influence sustained delivery parameters when the final precipitate contained zinc sulfate as a lyophilized salt (E and G). Neither Precipitates E nor G performed as well as Precipitate B in this vehicle, even though total zinc content was similar. This indicates that the manner (e.g., how it was precipitated or sprayed) in which zinc is incorporated into the precipitate significantly influences the sustained delivery of the peptide.

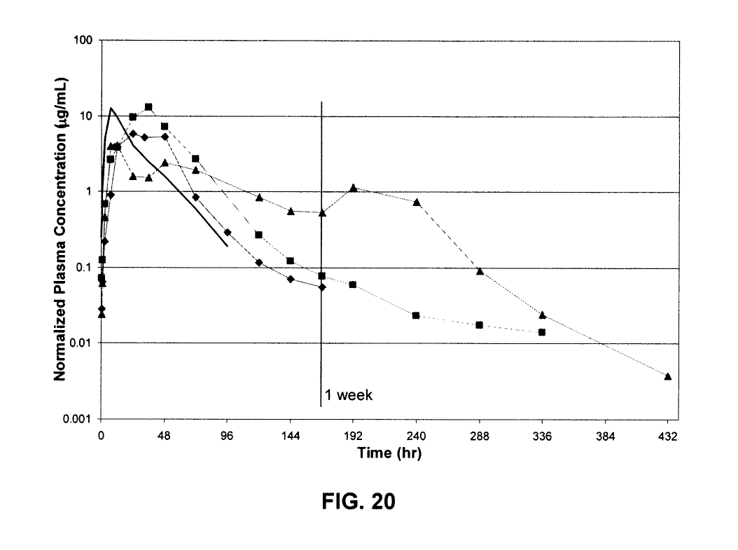

Influence of Polymer Type and Dose Volume on Pharmacokinetic Profiles in Monkeys. Precipitate D was formulated in SAIB:PLA:NMP vehicles and dosed in cynomolgus monkeys. As shown in FIG. 20, changing the vehicle from 75:5:20 to 74:11:15 (400 μL dose) did not significantly affect sustained delivery parameters; however, both performed better than the aqueous solution. Increasing dose volume to 1000 μL in the 74:11:15 decreased C_{MAX}, decreased t_{MAX} and increased t_{0.01}. This indicates that varying dose volume can influence sustained delivery.

### PLA and PLGA Gel Systems

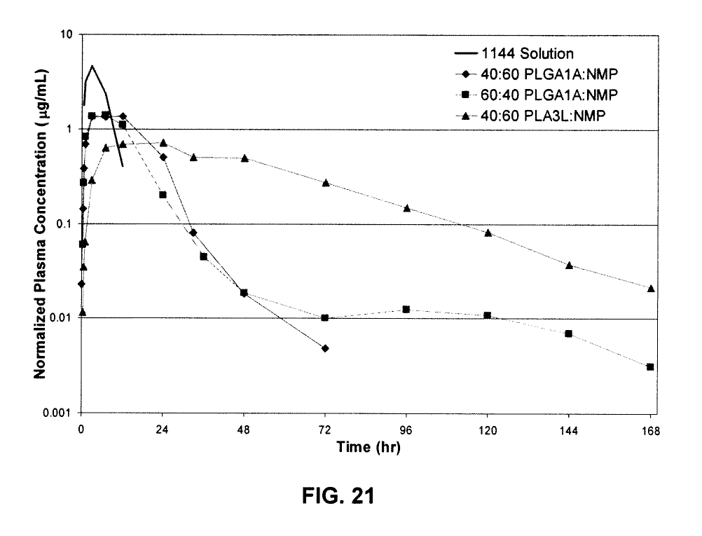

Influence of Matrix:Solvent Ratio on Pharmacokinetic Profiles in Rat. Precipitates A and D were formulated separately at 50 mg/g in PLGA1A:NMP vehicles and dosed at 400 μL in rats. As shown in FIG. 21, increasing matrix:solvent ratio (more PLGA) decreased C_{MAX}, increased t_{MAX} and increased t_{0.01}. This indicates that the amount of polymer in the vehicle influences sustained delivery.

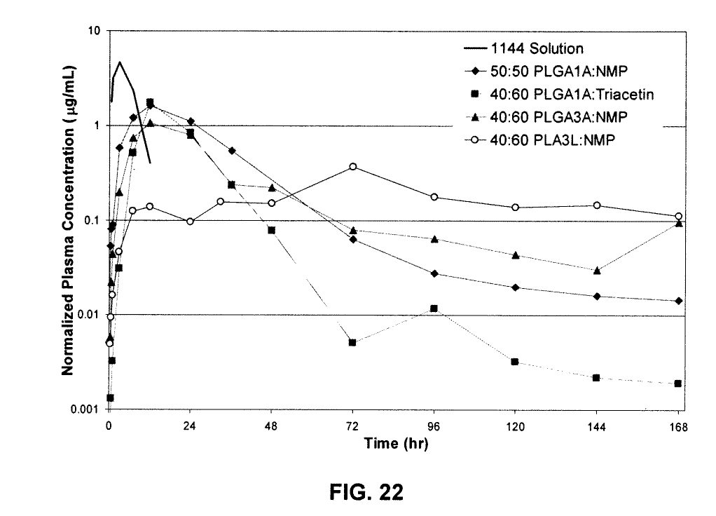

Influence of Polymer Type on Pharmacokinetic Profiles in Rat. Precipitates A and D were formulated separately at 50 mg/g in polymer:NMP vehicles and dosed at 400 μL in rats. As shown in FIG. 21, increasing polymer MW and L:G ratio simultaneously decreased C_{MAX}, increased t_{MAX} and increased t_{0.01}. Precipitate B was formulated at 50 mg/g in polymer:NMP vehicles and dosed at 400 μL in rats. As shown in FIG. 22, increasing L:G ratio decreased C_{MAX}, increased t_{MAX} and increased t_{0.01}. Increasing polymer MW decreased C_{MAX}, increased t_{MAX} and increased t_{0.01}. This indicates that polymer type in the vehicle influences sustained delivery.

Influence of Solvent Type on Pharmacokinetic Profiles in Rat. Precipitate B was formulated at 50 mg/g in PLGA1A:solvent vehicles and dosed at 400 μL in rats. As shown in FIG. 22, changing solvents from NMP to triacetin only increased t_{0.01}. This indicates that solvent type in the vehicle influences sustained delivery.

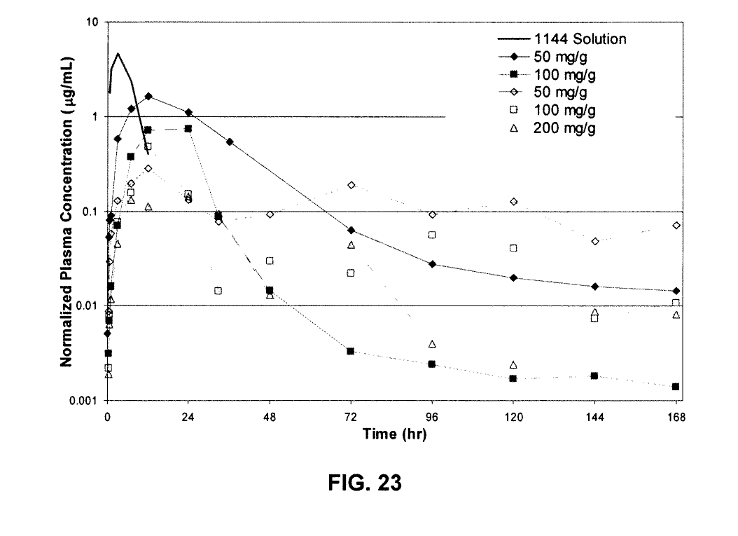

Influence of Peptide Concentration on Pharmacokinetic Profiles in Rat. Precipitate B was formulated in 50:50 PLGA1A:NMP vehicles and dosed at 400 μL in rats. As shown in FIG. 23, increasing dose decreased C_{MAX}, increased t_{MAX} and decreased t_{0.01} (all normalized). Precipitate H was formulated in 40:60 PLA3L:NMP vehicles and dosed at 400 μL in rats. As shown in FIG. 23, increasing dose decreased C_{MAX}, increased t_{MAX} and decreased t_{0.01} (all normalized). This indicates that PLA and PLGA vehicles can provide sustained delivery of high doses of peptides.

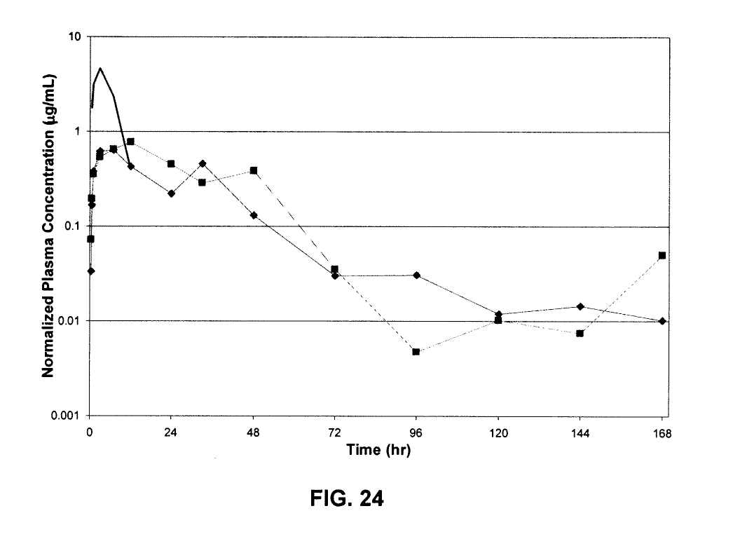

Influence of Peptide Form (no zinc) on Pharmacokinetic Profiles in Rat. Precipitate I and a spray-dried material were formulated at 50 mg/g in a 40:60 PLA3L:NMP vehicle and dosed at 400 μL in rats. As shown in FIG. 24, changing non-zinc containing peptide forms did not change sustained delivery parameters. This indicates that the physical form of the peptide without zinc does not significantly influence sustained delivery.

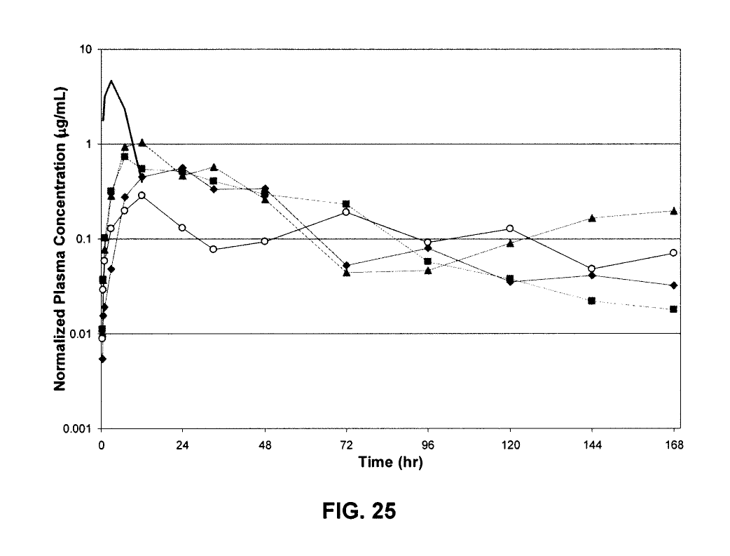

Influence of Peptide Form on Pharmacokinetic Profiles in Rat. Precipitates H, J, K and L were formulated at 50 mg/g in a 40:60 PLA3L:NMP vehicle and dosed at 400 μL in rats. As shown in FIG. 25, washing the precipitate, thereby decreasing zinc content, did not change sustained delivery parameters (J and L). The washed precipitate did perform in a manner significantly different from an unwashed low-zinc precipitate (K and L). Precipitating from a 50:50 methanol:water solution caused decreased C_{MAX} and increased t_{MAX}. This indicates that in this vehicle, the amount of zinc precipitated with the peptide does not influence sustained delivery; however, the solution from which the peptide is precipitated significantly affects delivery.

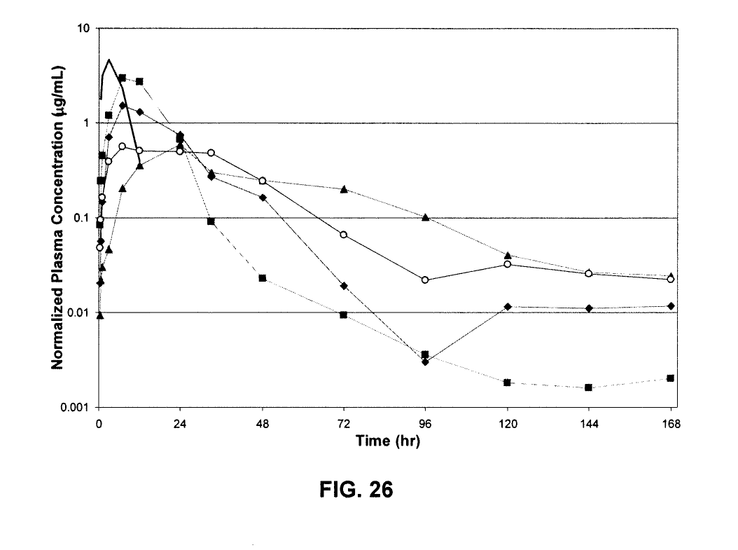

Influence of Cation Type on Pharmacokinetic Profiles in Rat. Several calcium and iron precipitates were formulated in a 40:60 PLA3L:NMP vehicle and dosed at 400 μL in rats. As shown in FIG. 26, all formulations exhibited some sustained delivery, with the iron formulations performing better than the calcium formulations. This indicates that other cation precipitates in addition to zinc can provide sustained delivery of peptides.

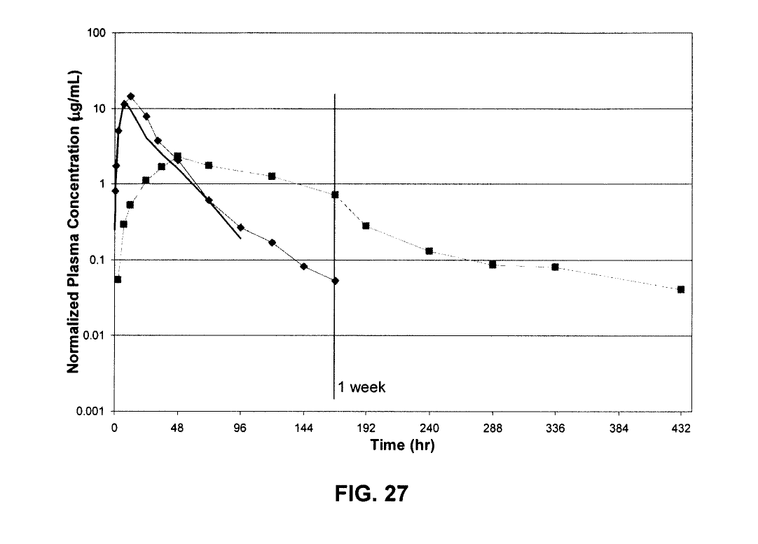

Influence of Polymer Type and Peptide Form on Pharmacokinetic Profiles in Monkeys. Precipitates were formulated in polymer:NMP vehicles and dosed in cynomolgus monkeys. As shown in FIG. 27, simultaneously changing from 40:60 PLGA1A:NMP vehicle and Precipitate A to 40:60 PLA3L:NMP vehicle and Precipitate J decreased C_{MAX}, decreased t_{MAX} and increased t_{0.01}. This indicates that polymer properties (e.g., type and molecular weight) and the manner in which the peptide was prepared are important in sustained delivery.

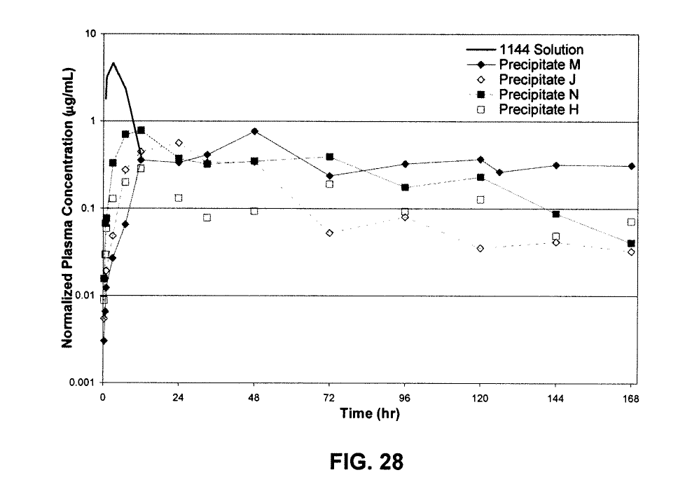

Influence of Precipitation Method on Pharmacokinetic Profiles in Rat. Precipitates H, J, M and N were formulated separately in a 40:60 PLA3L:NMP vehicle and dosed at 400 μL in rats. As shown in FIG. 28, standard Precipitates J and H are analogous (i.e., have similar peptide and zinc content) to sprayed Precipitates M and N, respectively. In each case, the sprayed precipitate exhibited increased C_{MAX} and t_{MAX}. Plasma concentration at the end of the week for the sprayed precipitate was greater than or equivalent to that of the standard precipitate. This indicates that sprayed precipitates can provide improved sustained delivery potential compared to standard precipitates.

### In-Situ Forming Gels

The following examples illustrate the preparation of in situ forming gel formulations comprising the peptide of the invention. In the following examples, the in situ gel formulates were based on the Atrigel technology from Atrix Pharmaceuticals and the SABERT™ technology from DURECT Pharmaceuticals.

In one example, the drug substance is suspended in a thermoplastic system in which a solid, linear-chain, biodegradable polymer or copolymer, such as poly-lactide-co-glycolide acid (PLGA), is dissolved in a solvent, such as N-methylpyrrolidinone(NMP) to form a liquid solution. Once the polymer solution is placed into the body where there is sufficient water, the solvent diffuses away from the polymer, leaving the polymer to solidify into a solid structure. As the PLGA degrades, the drug substance is slowly released. An example of a commercial product is Eligard® or leuprolide acetate suspended in Atrigel®.

In another example, using the SABER technology, the drug substance is suspended in sucrose acetate isobutyrate(SAIB) which has been mixed with ethanol to lower the viscosity of the solution. SAIB is a non-polymeric, non-water soluble high-viscosity liquid material that does not crystallize under ambient or physiological conditions. Once the peptide-SAIB solution is placed into the body where there is sufficient water, the solvent diffuses away from the SAIB, leaving the SAIB to solidify into a solid structure. As the SAIB degrades, the drug substance is slowly released into the blood stream. An example of a commercial product that is useful is Posidur™ or bupivicaine suspended in SAIB.

### Example 16

Illustrated here are further examples of the preparation of in situ gel formulations comprising the peptides of the present invention. Initially, TRI-1144 was spray dried or precipitated by the addition of glacial acetic acid. TRI-1144 was then precipitated from aqueous solution with zinc sulfate to form a Zn:TRI-1144 complex. When analyzed for zinc and peptide contents, the Zn:TR-1144 ratio was 1.1:1. In other examples, TRI-1144 was dissolved in 50:50 water:methanol solution before precipitation by the addition of zinc sulfate.

SAIB was purchased from Mallinckrodt. The TRI-1144 precipitates were suspended in various SAIB:PLGA:Ethanol (Table 11) solutions before dosing into rats/monkeys. The PLGA was dissolved in Ethanol and then added to the SAIB to achieve the desired % wt-wt. Based on the results of the animal PK studies, the precipitate type and solvent were optimized.

TABLE 11
SAIB:PLGA:Ethanol Solvent Systems Studied (% Wt/Wt)

| SAIB | PLGA | Ethanol |
|---|---|---|
| 74 | 11 | 15 |
| 65 | 15 | 20 |
| 70 | 15 | 15 |
| 75 | 5 | 20 |

All PLA/PLGA polymers were obtained from Lakeshore Biomaterials, Mobile, Ala. The zinc precipitates were suspended in various PLA:NMP and PLGA:NMP solutions (Table 12) before dosing into rats/monkeys. In general, the solvents were prepared by dissolving the PLA/PLGA in NMP and then adding the other hydrophilic solvent when necessary. Based on the results of the animal PK studies, the precipitate type and solvent were optimized.

TABLE 12
PLA:NMP and PLGA:NMP Solvent Systems Studied

| PLA/PLGA Type | | NMP | Other Hydrophilic Solvent | |
|---|---|---|---|---|
| (% Wt-Wt) | | (% Wt-Wt) | (% Wt-Wt) | |
| PLA3L | 40 | 60 | — | |
| PLGA2A | 40 | 60 | — | |
| PLGA2M | 40 | 60 | — | |
| PLGA3A | 40 | 60 | — | |
| PLGA3E | 40 | 60 | — | |
| PLGA-PEG 1500 | 40 | 60 | — | |
| PLA-PEG 1500 | 40 | 60 | — | |
| PLGA3A | 40 | — | Benzyl Alcohol | 60 |
| PLGA3A | 40 | — | Glycofurol | 60 |
| PLGA3A | 40 | 12 | Ethyl Lactate | 48 |
| PLGA3A | 40 | — | Propyl Carbonate | 60 |
| PLGA3A | 40 | 12 | Ethyl Benzoate | 48 |
| PLGA3A | 40 | 30 | PEG400 | 30 |
| PLGA3A | 40 | 42 | Propylene Glycol | 18 |
| PLGA3A | 40 | 48 | Ethanol | 12 |
| PLGA3A | 40 | 54 | Water | 6 |
| PLA3L | 40 | — | Benzyl Alcohol | 60 |
| PLA3L | 30 | — | Benzyl Benzoate | 70 |
| PLA3L | 30 | — | Triacetin | 70 |
| PLA3L | 30 | — | Glycofurol | 70 |
| PLA 3L | 40 | — | Ethyl Lactate | 60 |
| PLA 3L | 40 | — | Propyl Carbonate | 60 |
| PLA3L | 40 | 36 | Ethanol | 24 |
| PLA3L | 30 | 35 | PEG400 | 35 |
| PLA 3L | 40 | 42 | Propylene Glycol | 18 |
| PLA3L | 40 | 51 | Miglyol | 9 |
| PLGA-PEG1500 | 30 | 70 | — | |
| PLGA-PEG1500 | 20 | 80 | — | |
| PLA-PEG1500 | 20 | 80 | — | |
| PLGA2.5A | 40 | 30 | PEG400 | 30 |
| PLGA2A | 40 | 30 | PEG400 | 30 |
| PLGA1A | 40 | 30 | PEG400 | 30 |
| PLGA-PEG1500 | 20 | 40 | PEG400 | 40 |
| PLGA-PEG1500 | 10 | 45 | PEG400 | 45 |

TABLE 13
Index for Animal Pharmacokinetic Study

| Animal | | |
|---|---|---|
| PK Study # | Animal | Purpose |
| 385 | Monkey | TRI-1144 PLGA Microspheres |
| 386 | Monkey | TRI-1144 Insoluble Salt - Oil Suspension |
| 392 | Rat | TRI-1144 Insoluble Salt - Oil Suspension |
| 393 | Monkey | TRI-1144 Insoluble Salt - Oil Suspension |
| 400 | Rat | TRI-1144 Insoluble Salt - Oil Suspension |
| 401 | Rat | TRI-1144 Insoluble Salt - Oil Suspension |
| | | TRI-1144 Microspheres |
| 409 | Rat | TRI-1144 Microsphere Burst Evaluation |
| 427 | Rat | TRI-1144 with Phase Transfer Cations |
| 433 | Monkey | TRI-1144 Insoluble Salt - Oil Suspension |
| 446 | Rat | TRI-1144 Insoluble Salt - Oil Suspension |
| 462 | Rat | TRI-1144 Insoluble Salt - Oil Suspension |
| 464 | Rat | TRI-1144 with Phase Transfer Cations |
| 482 | Rat | TRI-1144 with Phase Transfer Cations |
| 490 | Rat | In Situ forming Gels with SAIB |
| 494 | Rat | TRI-1144 with Phase Transfer Cations |
| 507 | Rat | In Situ Forming Gels with SAIB |
| | | In Situ Forming Gels with PLGA:NMP |
| 520 | Rat | In Situ Forming Gels with SAIB |
| | | In Situ Forming Gels with PLGA:NMP |
| 525 | Rat | In Situ Forming Gels with SAIB Solvent |
| | | Optimization |
| 526 | Rat | In Situ Forming Gels with PLA:NMP, Initial 1 |
| | | week release from Zn:TRI-1144 Precipitate, |
| | | “high Zn” |
| 527 | Monkey | In Situ Forming Gels with PLA:NMP, Initial 1 |
| | | week release from Zn:TRI-1144 Precipitate, |
| | | “high Zn” |
| 530 | Rat | In Situ Forming Gels with PLA:NMP, 1 week |
| | | release from Zn:TRI-1144 Precipitate, “low Zn” |
| 531 | Rat | In Situ Forming Gels with PLA:NMP, Initial 1 |
| | | week release from Zn:TRI-1144 Precipitated |
| | | from Methanol:Water with “low Zn” |
| 540 | Rat | In Situ Forming Gels with PLA:NMP |
| | | Aqueous Suspension of Zn:TRI-1144 |
| | | Precipitate, “low Zn” |
| 552 | Rat | In Situ Forming Gels with PLGA Optimization |
| 553 | Rat | In Situ Forming Gels with PLA-PEG 1500 and |
| | | PLGA-PEG 1500 Co-polymers:NMP |
| 560 | Rat | In Situ Forming Gels Hydrophilic Solvent |
| | | Optimization |
| 561 | Rat | In Situ Forming Gels Hydrophilic Solvent |
| | | Optimization |
| 585 | Rat | In Situ Forming Gels Hydrophilic |
| | | Solvent/PLGA Optimization |

### RESULTS/DISCUSSION

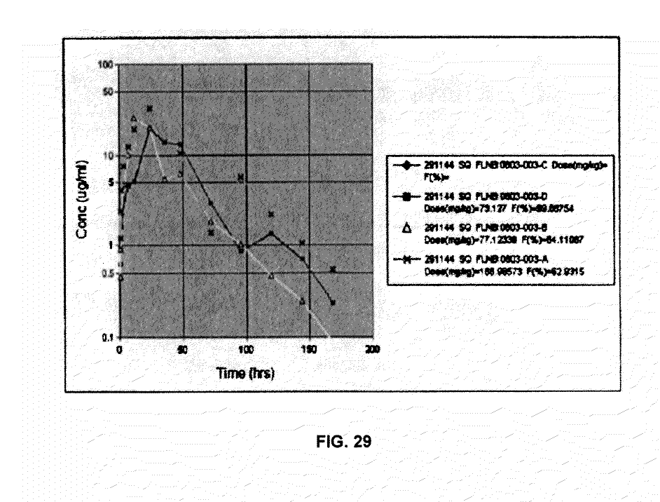

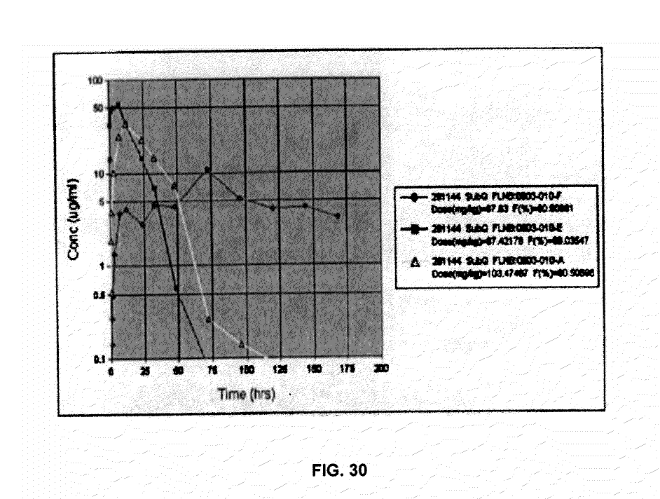

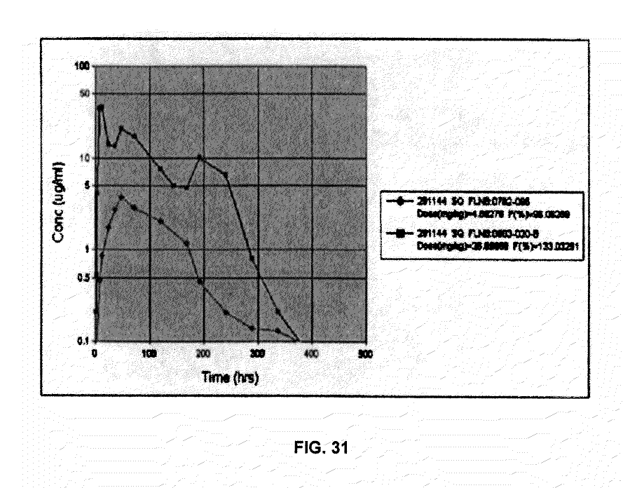

The in situ forming gel of TRI-1144 formed with SAIB demonstrated the first one week sustained release. FIG. 29 shows the TRI-1144 release in rats of TR-1144 SAIB gels. As the concentration of the SAIB is increased, the longer TRI-1144 is released. However, when compared to the in situ forming gels of TRI-1144 in PLA/PLGA:NMP, the release of TRI-1144 is not as long. FIG. 30 shows the comparison of in situ forming gels formed by Atrigel system vs SABER. The SABER gels have a relatively higher burst and essentially all of the TRI-1144 is released over 100 hours. However, TRI-1144 gels formed with the Atrigel system are still releasing TRI-1144 after one week. Also, the TRI-1144 formulations using the Atrigel technology were less viscous than the formulations using the SABER technology. This is evident in the results of the Monkey PK Study #527 as shown in FIG. 31. The TRI-1144 is released over the same period for both formulations. However the TRI-1144 suspended in the PLGA is dosed much easier due to its relatively lower viscosity.

### Zn-TRI-1144 Precipitate Optimization

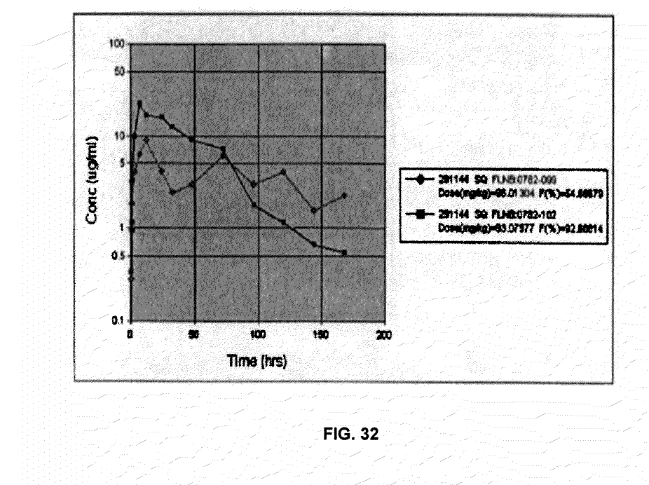

The sustained release of TRI-1144 is further increased by precipitating TRI-1144 with zinc sulfate. When analyzed for zinc content, zinc complexes with TRI-1144 in a 1.1:1 ratio. Initial formulations contained up to a 100 fold access of zinc. The results shown in the monkey PK Study #in FIG. 31 and from the results of Formulation 782-102 from Rat PK Study #530 in FIG. 8 indicate that the sustained release is enhanced by the Zn-TRI-1144 complex. However, the results are similar when minimum amount of zinc is used to complex with TRI-1144 as shown in the results for Formulation 782-099 from Rat PK Study #531 in FIG. 32. The Zn-TRI-1144 precipitate was further optimized by dissolving TRI-1144 in a 50:50 water:methanol solution before precipitating with zinc sulfate. Where the precipitate from the precipitation of TR-1144 dissolved in water is a fine white, flowing powder, the Zn-TRI-1144 precipitate formed when TRI-1144 is dissolved in the organic solution is a chunky solid that needs milling before suspension in the dosing solvent. When analyzed by x-ray powder diffraction, the Zn-TRI-1144 precipitate from the water solution is amorphous and the precipitate from the organic solution has approximately 10% crystalline.

### PLA/PLGA Optimization

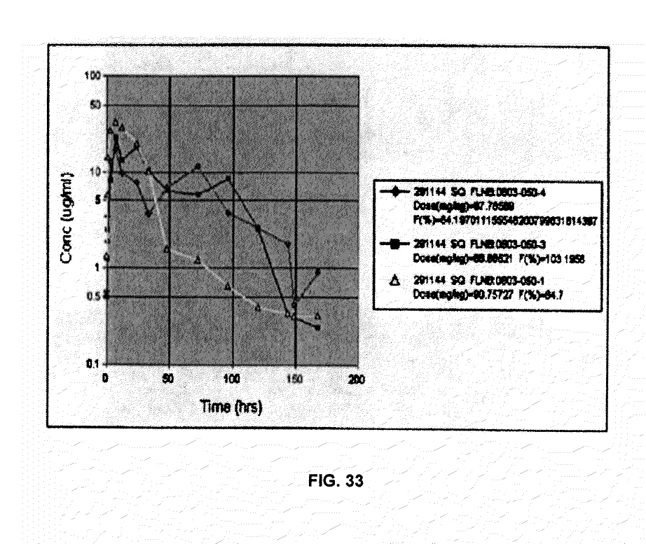

The polymer chain length and type were also optimized. In general, PLA has a longer sustained release than PLGA, and the polymers with the longer chain lengths have longer sustained release. This is directly proportional to the polymer degradation and solution viscosity of the dosing solution. In FIG. 33, the formulation where PLGA2A is dissolved in NMP has a higher burst and less of a sustained release when compared to the formulations using PLGA2.5A and PLGA3A.

### In Situ Forming Gels Using PLA-PEG1500 and PLGA-PEG 1500

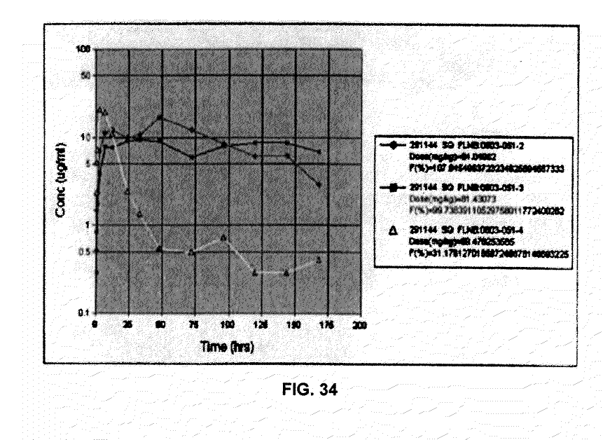

In situ forming gel formulations of Zn-TRI-1144 were also prepared using copolymers of PLA with PEG 1500 and PLGA with PEG 1500. The theory is that PEG 1500 will allow the polymer to be more hydrophilic. This would allow for quicker dissipation of the NMP and, thus, assist in the setting of the gel. The results from Rat PK Study #shown in FIG. 34 demonstrate a zero order release over one week of TRI-1144 from the in situ forming gels using PLA-PEG and PLGA-PEG 1500 co-polymers. These formulations were the first to demonstrate the feasibility of a once/week dose formulation of TRI-1144.

### Hydrophilic Solvent Optimization

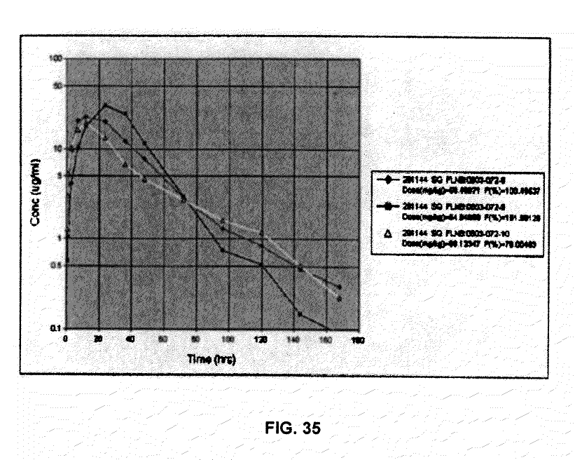

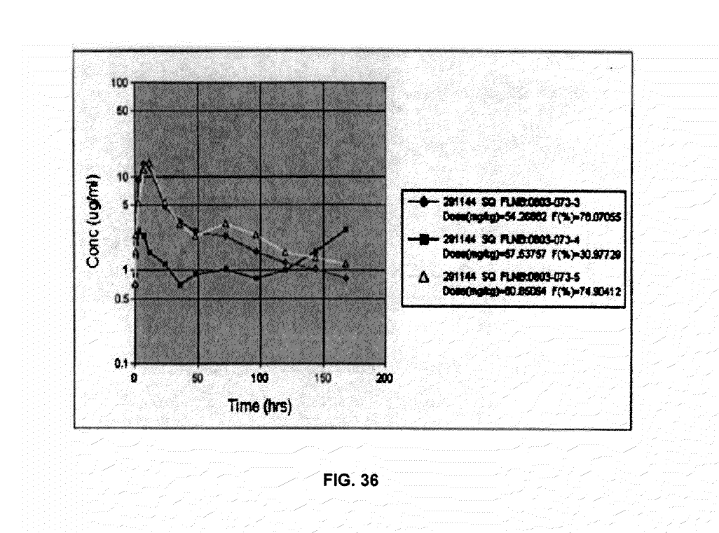

Though NMP is an acceptable solvent for subcutaneous injection, the amount of NMP in the formulations studied thus far is above the pharmaceutically acceptable limit for once/day dosing and potentially for once/week dosing. Therefore, based on the results from Rat PK Study #553 where the PLA-PEG1500 and PLGA-PEG1500 co-polymers were used to form the in situ forming gel, a series of studies were designed to optimize other hydrophilic solvents that with MP would still dissolve the PLA/PLGA polymers. The result from Rat PK Studies #560 and #561 shown in FIGS. 35 and 36, respectively, show that the addition of a hydrophilic solvent to the PLA/PLGA diluent enables the in situ forming gel to set quicker and, thus, sustain the release of TRI-1144. Of the solvents studied, PEG 400 appeared to be the optimum solvent researched (FIG. 36, 803-073-3 and 803-073-4).

### PLGA Optimization

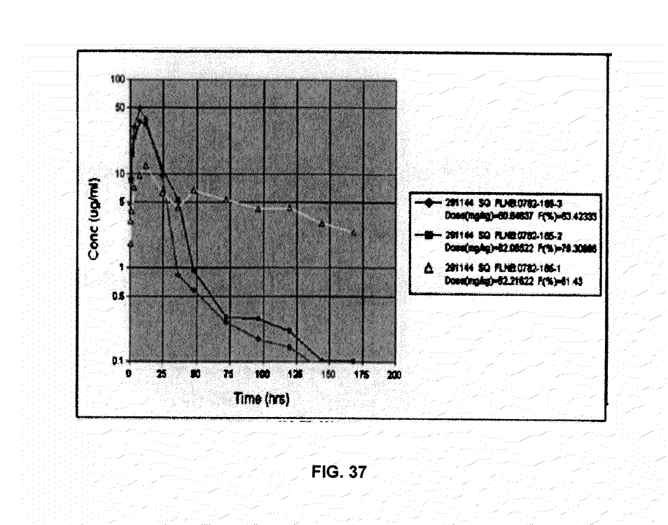

PLA 3L and PLGA 3A were initially used in the studies to optimize the solvent system due to PLA 3L and PLGA 3A having better sustained release. Any differences in the release of TRI-1144 because of the different solvent system would be easier to discern. However, PLA 3L degrades over 6 months and PLGA 3A degrades over one month, both of which would be unacceptable for a once/week dosing of TRI-1144. The solvent system optimized in Rat PK Studies #560 and #561 were the systems that contained PLA 3UPLGA 3A dissolved in NMP:PEG 400 (50:50) in a 40:60% wt/wt ratio. A faster degrading polymer is necessary. FIG. 37 shows the results of Rat PK Study #585. In this study, the formulations contained PLGA IA, PLGA 2A and PLGA 2.5A dissolved in NMP:PEG 1500. The in situ forming gels using PLGA 1A and PLGA 2A have similar results and are not feasible for once/week dosing of TRI-1144. The in situ forming gel using PLGA 2.5A has pseudo-first order release over one week and is acceptable for once/week dosing of TRI-1144.

The sustained release formulation of TRI-1144 using single- and double emulsions need to be researched further to determine feasibility for a once/week dosing of TR-1144. The loading of the peptide prevented the proper formation of the microspheres. When analyzed in vivo, these formulations resulted in a burst of TRI-1144 followed by a slow release of TRI-1144. These formulations in vivo release profiles were comparable to the TRI-1144 immediate release liquid formulation. A prototype formulation for once/week dosing of TRI-1144 was achieved by using in situ forming gel of Zinc-TRI-1144 precipitate suspended in a 50:50 PLGA 2.5A dissolved in 50:50 NMP:PEG 400. The 50:50 PLGA 2.5A will remain at the injection site for two weeks. The NMP:PEG 400 solvent system is within the pharmaceutically acceptable limits for NMP.

The foregoing description of the specific embodiments provided herein has been described in detail for purposes of illustration. In view of the descriptions and illustrations, others skilled in the art can, by applying current knowledge, readily modify and/or adapt the embodiments for various applications without departing from the basic concept; and thus, such modifications and/or adaptations are intended to be within the meaning and scope of the appended claims.

Various references are cited herein, the disclosures of which are incorporated by reference in their entireties.

What is claimed is:

1. A method of synthesis of a peptide comprising the amino acid sequence of SEQ ID NO:9, said method comprises condensation of peptide fragments, said peptide fragments comprise an amino acid sequence selected from the group consisting of SEQ ID NO: 58-77.

2. The method of claim 1 wherein said method comprises condensation of two peptide fragments.

3. The method of claim 2 wherein said peptide fragments comprise the amino acid sequences of SEQ ID NOS:72 and 58; SEQ ID NOS:70 and 59; SEQ ID NOS:64 and 60; or SEQ ID NOS:63 and 62.

4. The method of claim 1 wherein the synthesis is performed using rink-loaded CTC resin, Sieber resin, Ramage resin, Glu-loaded CTC resin, or Glu37 side-chain loaded resin.

5. The method of claim 1, further comprising the step of deprotection to form an acetyl group at the N-terminal of the peptide.

6. The method of claim 1, further comprising the step of decarboxylation to form an amido group at the C-terminal of the peptide.

7. The method of claim 1, further comprising a step of deprotection to form an acetyl group at the N-terminal of the peptide; and a step of decarboxylation to form an amido group at the C-terminal of the peptide.

8. The method of claim 1 wherein said method comprises condensation of three peptide fragments.

9. The method of claim 8, wherein said peptide fragments comprise the amino acid sequences of SEQ ID NOS:72, 74 and 20; SEQ ID NOS:71, 75, and 20; SEQ ID NOS:70, 76 and 20; or SEQ ID NOS:68, 77, and 20.

10. The method of claim 8 wherein the synthesis is performed using rink-loaded CTC resin, Sieber resin, Ramage resin, Glu-loaded CTC resin, or Glu37 side-chain loaded resin.

11. The method of claim 8, further comprising the step of deprotection to form an acetyl group at the N-terminal of the peptide.

12. The method of claim 8, further comprising the step of decarboxylation to form an amido group at the C-terminal of the peptide.

13. The method of claim 8, further comprising a step of deprotection to form an acetyl group at the N-terminal of the peptide; and a step of decarboxylation to form an amido group at the C-terminal of the peptide.

14. A method of synthesis of a peptide comprising SEQ ID NO:9, said method comprises condensation of peptide fragments, wherein said peptide fragments comprise the amino acid sequences of SEQ ID NO:72, 74 and 19; SEQ ID NO:71, 75, and 19; SEQ ID NO:70, 76 and 19; or SEQ ID NO:68, 77, and 19.

15. The method of claim 14, further comprises a step of covalently coupling the peptide fragment comprising an amino acid sequence of SEQ ID NO:19 with a leucine amino acid residue prior to condensation of the peptide fragments.

16. A method of synthesis of a peptide comprising SEQ ID NO:9, said method comprises condensation of two peptide fragments, wherein said peptide fragments comprise an amino acid sequence of SEQ ID NO:40 and SEQ ID NO:20, said synsthesis is performed using rink-loaded CTC resin, Sieber resin, or Glu-loaded CTC resin.

17. A method of synthesis of a peptide comprising SEQ ID NO:9, said method comprises linear solid phase synthesis using rink resin, modified acid resin, rink-linked-loaded CTC resin, or gamma-glutamyl(Leu-amido)-loaded CTC resin.

18. A set of peptide fragments for the synthesis of SEQ ID NO:9, comprising a set selected from the group consisting of:

| | |
|---|---|
| (a) | Ac-AA(1-26)-OH |
| and | |
| | AA(27-37)-Rink-OH; |
| (b) | Ac-AA(1-26)-OH |
| and | |
| | AA(27-38)-Rink-OH; |
| (c) | Ac-AA(1-12)-0H, Fmoc-AA(13-26)-0H, |
| and | |
| | AA(27-37)-Rink-OH; |
| (d) | Ac-AA(1-12)-0H; Fmoc-AA(13-26)-0H, |
| and | |
| AA(27-38)-Rink-OH; | |
| or | |
| (e) | Ac-AA(1-26)-OH and H-AA(27-38)-NH2 free Glu37. |

19. A peptide consisting of the amino acid sequence of SEQ ID NO:58, 59, 60, 61, 62, 63, 64, 65, 66, 67, 68, 69, 70, 71, 72, 73, 74, 75, 76, or 77.


SEQUENCE LISTING (80 sequences)

SEQ ID NO: 1 - protein, 64 aa, Artificial Sequence
  synthesized peptide
WNASWSNKSLEQIWNNMTWMEWDREINNYTSLIHSLIEESQNQQEKNEQELLELDKWASLWNWF

SEQ ID NO: 2 - protein, 36 aa, Artificial Sequence
  synthesized peptide
YTSLIHSLIEESQNQQEKNEQELLELDKWASLWNWF

SEQ ID NO: 3 - protein, 36 aa, Artificial Sequence
  synthesized peptide
MTWMEWDREINNYTSLIHSLIEESQNQQEKNEQELL

SEQ ID NO: 4 - protein, 36 aa, Artificial Sequence
  synthesized peptide
WMEWDREINNYTSLIHSLIEESQNQQEKNEQELLEL

SEQ ID NO: 5 - protein, 38 aa, Artificial Sequence
  synthesized peptide
TTWEAWDRAIAEYAARIEALIRAAQEQQEKNEAALREL

SEQ ID NO: 6 - protein, 38 aa, Artificial Sequence
  synthesized peptide
TTWEAWDRAIAEYAARIEALIRALQEQQEKNEAALREL

SEQ ID NO: 7 - protein, 38 aa, Artificial Sequence
  synthesized peptide
TTWEAWDRAIAEYAARIEALIRAAQEQQEKLEAALREL

SEQ ID NO: 8 - protein, 38 aa, Artificial Sequence
  synthesized peptide
TTWEAWDRAIAEYAARIEALLRAAQEQQEKNEAALREL

SEQ ID NO: 9 - protein, 38 aa, Artificial Sequence
  synthesized peptide
TTWEAWDRAIAEYAARIEALLRALQEQQEKNEAALREL

SEQ ID NO: 10 - protein, 38 aa, Artificial Sequence
  synthesized peptide
TTWEAWDRAIAEYAARIEALLRAAQEQQEKLEAALREL

SEQ ID NO: 11 - protein, 38 aa, Artificial Sequence
  synthesized peptide
TTWEAWDRAIAEYAARIEALIRALQEQQEKLEAALREL

SEQ ID NO: 12 - protein, 38 aa, Artificial Sequence
  synthesized peptide
TTWEAWDRAIAEYAARIEALIRAIQEQQEKLEAALREL

SEQ ID NO: 13 - protein, 38 aa, Artificial Sequence
  synthesized peptide
TTWEAWDRAIAEYAARIEALIRALQEQQEKIEAALREL

SEQ ID NO: 14 - protein, 38 aa, Artificial Sequence
  synthesized peptide
TTWEAWDRAIAEYAARIEALLRAIQEQQEKNEAALREL

SEQ ID NO: 15 - protein, 38 aa, Artificial Sequence
  synthesized peptide
TTWEAWDRAIAEYAARIEALLRAAQEQQEKIEAALREL

SEQ ID NO: 16 - protein, 38 aa, Artificial Sequence
  synthesized peptide
XXXEAXDRAXAEXAARXEAXXRAXXEXXEKXEAAXREX

SEQ ID NO: 17 - protein, 12 aa, Artificial Sequence
  synthesized peptide
TTWEAWDRAIAE

SEQ ID NO: 18 - protein, 14 aa, Artificial Sequence
  synthesized peptide
YAARIEALLRALQE

SEQ ID NO: 19 - protein, 11 aa, Artificial Sequence
  synthesized peptide
QQEKNEAALRE

SEQ ID NO: 20 - protein, 12 aa, Artificial Sequence
  synthesized peptide
QQEKNEAALREL

SEQ ID NO: 21 - protein, 11 aa, Artificial Sequence
  synthesized peptide
TTWEAWDRAIA

SEQ ID NO: 22 - protein, 15 aa, Artificial Sequence
  synthesized peptide
EYAARIEALLRALQE

SEQ ID NO: 23 - protein, 10 aa, Artificial Sequence
  synthesized peptide
TTWEAWDRAI

SEQ ID NO: 24 - protein, 16 aa, Artificial Sequence
  synthesized peptide
AEYAARIEALLRALQE

SEQ ID NO: 25 - protein, 9 aa, Artificial Sequence
  synthesized peptide
TTWEAWDRA

SEQ ID NO: 26 - protein, 17 aa, Artificial Sequence
  synthesized peptide
IAEYAARIEALLRALQE

SEQ ID NO: 27 - protein, 8 aa, Artificial Sequence
  synthesized peptide
TTWEAWDR

SEQ ID NO: 28 - protein, 18 aa, Artificial Sequence
  synthesized peptide
AIAEYAARIEALLRALQE

SEQ ID NO: 29 - protein, 20 aa, Artificial Sequence
  synthesized peptide
TTWEAWDRAIAEYAARIEAL

SEQ ID NO: 30 - protein, 17 aa, Artificial Sequence
  synthesized peptide
LRALQEQQEKNEAALRE

SEQ ID NO: 31 - protein, 18 aa, Artificial Sequence
  synthesized peptide
LRALQEQQEKNEAALREL

SEQ ID NO: 32 - protein, 18 aa, Artificial Sequence
  synthesized peptide
TTWEAWDRAIAEYAARIE

SEQ ID NO: 33 - protein, 19 aa, Artificial Sequence
  synthesized peptide
ALLRALQEQQEKNEAALRE

SEQ ID NO: 34 - protein, 20 aa, Artificial Sequence
  synthesized peptide
ALLRALQEQQEKNEAALREL

SEQ ID NO: 35 - protein, 26 aa, Artificial Sequence
  synthesized peptide
YAARIEALLRALQEQQEKNEAALREL

SEQ ID NO: 36 - protein, 27 aa, Artificial Sequence
  synthesized peptide
EYAARIEALLRALQEQQEKNEAALREL

SEQ ID NO: 37 - protein, 28 aa, Artificial Sequence
  synthesized peptide
AEYAARIEALLRALQEQQEKNEAALREL

SEQ ID NO: 38 - protein, 29 aa, Artificial Sequence
  synthesized peptide
IAEYAARIEALLRALQEQQEKNEAALREL

SEQ ID NO: 39 - protein, 30 aa, Artificial Sequence
  synthesized peptide
AIAEYAARIEALLRALQEQQEKNEAALREL

SEQ ID NO: 40 - protein, 26 aa, Artificial Sequence
  synthesized peptide
TTWEAWDRAIAEYAARIEALLRALQE

SEQ ID NO: 41 - protein, 14 aa, Artificial Sequence
  synthesized peptide
YAARIEALLRAAQE

SEQ ID NO: 42 - protein, 11 aa, Artificial Sequence
  synthesized peptide
QQEKLEAALRE

SEQ ID NO: 43 - protein, 12 aa, Artificial Sequence
  synthesized peptide
QQEKLEAALREL

SEQ ID NO: 44 - protein, 15 aa, Artificial Sequence
  synthesized peptide
EYAARIEALLRAAQE

SEQ ID NO: 45 - protein, 16 aa, Artificial Sequence
  synthesized peptide
AEYAARIEALLRAAQE

SEQ ID NO: 46 - protein, 17 aa, Artificial Sequence
  synthesized peptide
IAEYAARIEALLRAAQE

SEQ ID NO: 47 - protein, 18 aa, Artificial Sequence
  synthesized peptide
AIAEYAARIEALLRAAQE

SEQ ID NO: 48 - protein, 17 aa, Artificial Sequence
  synthesized peptide
LRAAQEQQEKLEAALRE

SEQ ID NO: 49 - protein, 19 aa, Artificial Sequence
  synthesized peptide
LRAALQEQQEKLEAALREL

SEQ ID NO: 50 - protein, 19 aa, Artificial Sequence
  synthesized peptide
ALLRAAQEQQEKLEAALRE

SEQ ID NO: 51 - protein, 20 aa, Artificial Sequence
  synthesized peptide
ALLRAAQEQQEKLEAALREL

SEQ ID NO: 52 - protein, 26 aa, Artificial Sequence
  synthesized peptide
YAARIEALLRAAQEQQEKLEAALREL

SEQ ID NO: 53 - protein, 27 aa, Artificial Sequence
  synthesized peptide
EYAARIEALLRAAQEQQEKLEAALREL

SEQ ID NO: 54 - protein, 28 aa, Artificial Sequence
  synthesized peptide
AEYAARIEALLRAAQEQQEKLEAALREL

SEQ ID NO: 55 - protein, 29 aa, Artificial Sequence
  synthesized peptide
IAEYAARIEALLRAAQEQQEKLEAALREL

SEQ ID NO: 56 - protein, 30 aa, Artificial Sequence
  synthesized peptide
AIAEYAARIEALLRAAQEQQEKLEAALREL

SEQ ID NO: 57 - protein, 37 aa, Artificial Sequence
  synthetic peptide
TTWEAWDRAIAEYAARIEALLRALQEQQEKNEAALRE

SEQ ID NO: 58 - protein, 24 aa, Artificial Sequence
  synthetic peptide
AARIEALLRALQEQQEKNEAALRE

SEQ ID NO: 59 - protein, 22 aa, Artificial Sequence
  synthetic peptide
RIEALLRALQEQQEKNEAALRE

SEQ ID NO: 60 - protein, 14 aa, Artificial Sequence
  synthetic peptide
QEQQEKNEAALREL

SEQ ID NO: 61 - protein, 15 aa, Artificial Sequence
  synthetic peptide
LQEQQEKNEAALREL

SEQ ID NO: 62 - protein, 13 aa, Artificial Sequence
  synthetic peptide
EQQEKNEAALREL

SEQ ID NO: 63 - protein, 25 aa, Artificial Sequence
  synthetic peptide
TTWEAWDRAIAEYAARIEALLRALQ

SEQ ID NO: 64 - protein, 24 aa, Artificial Sequence
  synthetic peptide
TTWEAWDRAIAEYAARIEALLRAL

SEQ ID NO: 65 - protein, 22 aa, Artificial Sequence
  synthetic peptide
TTWEAWDRAIAEYAARIEALLR

SEQ ID NO: 66 - protein, 21 aa, Artificial Sequence
  synthetic peptide
TTWEAWDRAIAEYAARIEALL

SEQ ID NO: 67 - protein, 19 aa, Artificial Sequence
  synthetic peptide
TTWEAWDRAIAEYAARIEA

SEQ ID NO: 68 - protein, 17 aa, Artificial Sequence
  synthetic peptide
TTWEAWDRAIAEYAARI

SEQ ID NO: 69 - protein, 16 aa, Artificial Sequence
  synthetic peptide
TTWEAWDRAIAEYAAR

SEQ ID NO: 70 - protein, 15 aa, Artificial Sequence
  synthetic peptide
TTWEAWDRAIAEYAA

SEQ ID NO: 71 - protein, 14 aa, Artificial Sequence
  synthetic peptide
TTWEAWDRAIAEYA

SEQ ID NO: 72 - protein, 13 aa, Artificial Sequence
  synthetic peptide
TTWEAWDRAIAEY

SEQ ID NO: 73 - protein, 2 aa, Artificial Sequence
  synthetic peptide
EL

SEQ ID NO: 74 - protein, 13 aa, Artificial Sequence
  synthetic peptide
AARIEALLRALQE

SEQ ID NO: 75 - protein, 12 aa, Artificial Sequence
  synthetic peptide
ARIEALLRALQE

SEQ ID NO: 76 - protein, 11 aa, Artificial Sequence
  synthetic peptide
RIEALLRALQE

SEQ ID NO: 77 - protein, 9 aa, Artificial Sequence
  synthetic peptide
EALLRALQE

SEQ ID NO: 78 - protein, 39 aa, Artificial Sequence
  synthetic peptide
WQEWEQKITALLEQAQIQQEKNEYELQKLDKWASLWEWF

SEQ ID NO: 79 - protein, 39 aa, Artificial Sequence
  synthetic peptide
YTSLIHSLIEESQNQQEKNEQELLELDKWASLWNWFNIT

SEQ ID NO: 80 - protein, 36 aa, Artificial Sequence
  synthetic peptide
YQEWERKVDFLEENITALLEEAQIQQEKNMYELQKL